(19) United States
(12) Patent Application Publication
(10) Pub. No.: US 2011/0217265 A1
(43) Pub. Date: Sep. 8, 2011

(54) Screening for Inhibitors of HCV Amphipathic Helix (AH) Function

(76) Inventors: Jeffrey S. Glenn, Palo Alto, CA (US); Nam-Joon Cho, Stanford, CA (US); Wenjin Yang, Foster City, CA (US)

(21) Appl. No.: 13/119,245

(22) PCT Filed: Sep. 23, 2009
(86) PCT No.: PCT/US09/05306

§ 371 Date: May 20, 2011

Related U.S. Application Data
(60) Provisional application No. 61/099,505, filed on Sep. 23, 2008.

(51) Int. Cl. A61K 31/4965; A61K 31/497; A61K 31/7064; A61K 38/21; A61P 31/14; C40B 30/10; C40B 30/00; G01B 11/02

(52) U.S. Cl. 424/85.7; 514/255.05; 514/255.06; 514/43; 506/12; 506/7; 356/635

(57) ABSTRACT

Screening methods are provided for identifying pharmacologic inhibitors of HCV amphipathic helix (AH) function, which inhibitors are useful in the prevention and treatment of HCV infection. Also provided are compounds useful in the inhibition of viral replication. The methods of the invention are based on the unexpected discovery that the presence of an AH, e.g. an AH of an HCV polypeptide, causes an increase in the apparent diameter of the vesicles. The methods of the invention provide for addition of AH peptides to lipid vesicles, for example in a high-throughput format; which addition may be performed in the absence or presence of a candidate pharmacologic agent. The change in apparent vesicle size is measured, and compared to control samples. An increase in vesicle size or aggregation is indicative of AH function being present; and a lack of increase is indicative that the AH function is absent or has been inhibited by a test agent.

### BACKGROUND OF THE INVENTION

Hepatitis C Virus (HCV) is a global health problem with estimates of more than 2% of the world's population currently infected with the virus. One of the outstanding characteristics of HCV is its ability to establish chronic infections in 65-80% of infected patients. Chronic infection with HCV can lead to serious sequelae including chronic active hepatitis, cirrhosis and hepatocellular carcinoma—usually manifested 10, 20 and 25 years respectively after the initial infection. End stage liver disease from HCV has become the leading indication for liver transplantation in North America, and it has been suggested that there will be a 2-3 fold increase in liver transplantation in 10 years as a result of cirrhosis from hepatitis C.

Discovered in 1989, the virus, classified as a Flavivirus, has a 9.5 kilobase positive-strand RNA genome which encodes a single polypeptide of 3008-3037 amino acids long. Based on the genetic variability of the virus, which can be up to 30% at the nucleotide level, at least 6 genotypes and more than 30 subtypes have been identified. This variability has implications for vaccine and antiviral drug development. At present the only approved therapies are interferon, with or without ribavirin, which is not successful in many patients. There is therefore an urgent need to develop novel antivirals to treat HCV.

Many components of the HCV polyprotein and genome have been identified and characterized. The open reading frame (ORF) of HCV is flanked by a non-translated region at the 5′ end, and approximately 200 nucleotides at the 3′ end containing a poly-U tract and a highly conserved 98 base sequence. The core protein located at the N-terminal end of the ORF is the viral capsid protein. The core protein is released from the viral polypeptide by host proteases. In addition to binding to viral RNA, the core protein has also been shown to suppress apoptotic cell death.

Like other positive strand RNA viruses, HCV is believed to replicate in association with cytoplasmic membranes. In the case of HCV, the structures are termed the membranous web and are believed to be induced by the NS4B protein. NS4B is also required to assemble the other viral NS proteins within the apparent sites of RNA replication. The site of viral replication and assembly appears to intersect with host cell pathways of lipid trafficking and lipoprotein production. Amphipathic helices (AHs) have been identified in several HCV NS proteins that mediate membrane association and HCV replication.

Certain interactions of viral proteins with cell membranes have previously been described. For example, in poliovirus and Hepatitis A virus, the nonstructural protein 2C contains a membrane associating amphipathic helix (See Teterina, N. L., et al., J. Virol. (1997) 71:8962-8972 (poliovirus); and Kusov, Y. Y., et al., Arch. Virol. (1998) 143:931-944 (Hepatitis A). This membrane association appears to play a role in RNA synthesis in poliovirus (Paul, A. V., et al., Virol. (1994) 199:188-199). Replication complexes are localized on the host endoplasmic reticulum (ER) and Golgi in the case of poliovirus (Bienz, K., et al., J. Virol. (1992) 66:2740-2747), and infection with poliovirus induces rearrangements of membranes derived from host ER and Golgi (Schlegel, A., et al., J. Virol. (1996) 70:6576-6588).

The NS5A protein of HCV is also associated with membranes. It precise role has not been determined, but it has been shown to play a role in RNA binding, multiple host-protein interactions, and interferon resistance. Its N-terminal amphipathic helix has been shown to be critical for viral replication and membrane anchoring. It is also known that the Hepatitis C nonstructural 5A protein is a potent transcriptional activator (Kato, N., et al., J. Virol. (1997) 71:8856-8859); that amino terminal deletion mutants of Hepatitis C virus nonstructural protein NS5A function as transcriptional activators in yeast (Tanimoto, A., et al., Biochem. Biophys. Res. Commun. (1997) 236:360-364); and that this nonstructural protein physically associates with p53 and regulates p21/Waf1 gene expression in a p53 dependent manner (Majumder, M., et al., J. Virol. (2001) 75:1401-1407).

There is an ongoing need in the art for agents that treat HCV infection; and for methods of identifying candidate agents that are suitable for treating HCV infection. The present invention addresses this need.

### SUMMARY OF THE INVENTION

Screening methods are provided for identifying pharmacologic inhibitors of HCV amphipathic helix (AH) function, which inhibitors are useful in the prevention and treatment of HCV infection. The methods of the invention are based on the unexpected discovery that the presence of an AH, e.g. an AH of an HCV polypeptide, causes a large increase in dynamic light scattering (DLS) upon addition to lipid vesicles, which measures an increase in the apparent diameter of the vesicles. The methods of the invention provide for addition of AH peptides to lipid vesicles, for example in a high-throughput format; which addition may be performed in the absence or presence of a candidate pharmacologic agent. The change in DLS is measured and compared to control samples. An increase in DLS is indicative of AH function being present; and a lack of increase is indicative that the AH function is absent or has been inhibited by a test agent. In alternative embodiments, measurements of change in vesicle size are used in place of DLS, e.g. altered fluorescence, visual inspection, and the like.

A number of AH peptides have been identified in HCV, including helices present in the N-termini of NS5A and NS4B; and a non-terminal AH in NS4B. Peptides corresponding to these sequences, or other peptides having an AH function can be utilized in the screening methods of the invention. The use of the internal AH in NS4B, 4BAH2, is of particular interest for aggregation of vesicles.

Agents identified as active inhibitors of AH function are useful in the inhibition of infection, replication, or pathogenesis of Hepatitis C Virus in vitro or in vivo when introduced into a host cell containing said virus. Inhibitors of interest may, for example, exhibit an IC₅₀ in the range of from about 0.0001 nM to about 100 μM in an in vitro assay for at least one step in infection, replication, or pathogenesis of the virus. In another embodiment, the invention provides a method of preventing or treating HCV infection in a patient in need thereof, comprising administering to said patient an anti-HCV effective amount of an agent identified by the methods of the invention. Inhibitors of particular interest include pyrazine 2-carboxamide analogs, as described herein.

In another embodiment, the invention provides a pharmaceutical composition of one or more isolated agents identified by the methods of the invention, and a pharmaceutically acceptable carrier, diluent, excipient, or buffer.

### BRIEF DESCRIPTION OF THE DRAWINGS

FIG. 1. DLS-based monitoring for inhibitors of AH function. (a) Average size distribution increase measured upon addition of NS5A AH to lipid vesicles as a function of time. (b) Identification of small peptides capable of inhibiting NS5A AH function. The assay of (a) above was repeated in the presence of small candidate pharmacologic inhibitors, in this case small peptides. Note peptides 12 and 24 are identified as inhibitors of NS5A AH function, while peptides 1 and 4 have significantly less inhibition activity.

FIG. 2. NS5A AH-induced DLS changes reflect an AH-induced increase in lipid vesicle average size. Electron microscopy of POPC lipid vesicles: (A) alone; (B) after addition of NS5A AH peptide; and (C) after addition of negative control NH peptide. Note that quantitative analysis reveals that the AH-treated vesicles are larger in size and also can form multilamellar vesicles.

FIG. 3. The NS4B second amphipathic helix, 4BAH2, is essential for HCV genome replication. Two point mutations were introduced into the second amphipathic helix of NS4B (4BAH2) so as to disrupt its amphipathic nature. The replication potential of wild-type and 4BAH2 mutant high efficiency subgenomic HCV replicons was then tested in: (A) standard colony formation assays [1=wild type genotype 1b subgenomic replicons. 2=same replicon but with A51E and W55D mutations that disrupt the hydrophobic face of 4BAH2. 3=control replicon with lethal mutation in the NS5B polymerase gene (Elazar et al. 2004, J. Virol.)], and (B) transient luciferase reporter-linked replication assays. Note that an intact 4BAH2 is essential for HCV genome replication.

FIG. 4. The NS4B second amphipathic helix, 4BAH2 (“AH2” in figure), induces a large increase in apparent vesicle size as measured by DLS. The size distribution of POPC lipid vesicles was measured by DLS in the absence (left panel) or presence (right panel) of 4BAH2. Note the differences in scale of the x-axis between left and right panels, reflecting the dramatic increase in apparent vesicle size upon addition of the 4BAH2 peptide. Bar and thin lines represent the histogram and Gaussian distributions, respectively.

FIG. 5. The 4BAH2-induced changes in DLS reflect predominantly 4BAH2-induced aggregation of lipid vesicles. POPC lipid vesicles were extruded through 30 nm polycarbonate track-etched membrane and then analyzed by electron microscopy before (left panel) or after (right panel) addition of 4BAH2. Although most vesicles appear to retain their initial size, they are predominantly organized into large aggregates upon addition of 4BAH2. Note the extremely large size of the 4BAH2-induced aggregations, as indicated by the size calibration bar.

FIG. 6. The 4BAH2-induced aggregation of lipid vesicles can be readily visualized by fluorescence microcopy. POPC lipid vesicles were prepared with the addition of a fluorescent lipid. Although the vesicles are too small to be visualized with a fluorescent microscope (left panel), after addition of 4BAH2 (right panel), the 4BAH2-induced large aggregates of vesicles could be readily visualized.

FIG. 7. 4BAH2-induced aggregation of lipid vesicles can be used to screen for small molecule inhibitors of 4BAH2 function. The assay of FIG. 6 was extended to include the addition of small molecule candidate inhibitors. Examples of drug candidates that score positive in this assay are indicated in the bottom panels.

FIG. 8. Quantitative image analysis of 4BAH2-induced aggregation of lipid vesicles for high throughput screening. The assay of FIG. 6 can be performed in a 384 well format and quantitative analysis of the automated images can identify candidate inhibitors of 4BAH2 function. Note some of the wells at the right of the plate that were treated with compounds found to score positive in this type of assay.

FIG. 9. DLS assays on selected molecules identified to be positive in the 4BAH2-induced lipid vesicle aggregation assay. Selected molecules that scored positive or negative in the 384-well 4BAH2-induced lipid vesicle aggregation assay were analyzed by the DLS assay of FIG. 4. POPC=POPC lipid vesicles alone. AH2=POPC lipid vesicles, DMSO, and 4BAH2. The same condition was used for all of the other assays except for the inclusion of the indicated compounds. Compounds CZ, H8, and H10 were included as negative controls. The bottom panel represents the quantitative analysis of the corresponding wells in the 384 well assay performed as in FIG. 8. PC=POPC.

FIG. 10. Inhibitors of 4BAH2 function identified in the DLS and lipid vesicle aggregation assays can inhibit HCV genome replication. One of the hits identified in FIG. 9 was tested in standard HCV replication assays as in FIG. 3. Top panel reflects anti-HCV activity measured using a genotype 1 luciferase reporter linked high efficiency subgenomic HCV replicon. Bottom panel indicates corresponding Alamar blue assays for cell metabolism. Note that the EC50 for this compound is in the low micromolar range.

FIG. 11. 4BAH2 inhibitors can increase the anti-HCV activity of agents targeting other elements of HCV. The compound of FIG. 10 was tested in standard HCV replication assays as in FIG. 10 but in the presence of various concentrations of an NS3 protease inhibitor (SCH503034, “SCH”). Note that in these assays a genotype 2b luciferase reporter-linked HCV replicons was used, indicating the broad spectrum potential of the C4 compound against multiple HCV genotypes.

FIG. 12. An amphipathic alpha helical segment of NS4B, 4BAH2, promotes largescale vesicle aggregation as measured by dynamic light scattering (DLS), transmission electron microscopy (TEM), and atomic force microscopy (AFM). (A) Helix net diagram of amino acids 40-62 of NS4B that comprise 4BAH2 (depicted in the N-terminal to C-terminal direction from bottom to top). Hydrophobic portions are indicated in green. (B) Far-UV circular dichroism (CD) recording of a synthetic peptide corresponding to 4BAH2 confirms that the peptide has an alpha helical structure. 1-palmitoyl-2-oleoyl-sn-glycero-3-phosphocholine (POPC) lipid vesicles were extruded through a 30-nm polycarbonate track-etched membrane and their size distribution was measured by dynamic light scattering (DLS) in the (C) absence or (D) presence of 4BAH2, which reflects the dramatic increase in the size distribution upon addition of 4BAH2. (E) No such activity was observed with a control amphipathic helical peptide (4BAH1). The red bars represent the histogram of size distribution. Note the x-axis scale is separated into two linear size ranges (break between 700 nm to 7500 nm) in order to directly compare the dramatic increase in the average vesicle size distribution upon addition of the wild-type 4BAH2 peptide. Transmission electron microscopy studies were performed (TEM) (F) before and (G) after addition of 4BAH2 to investigate the size increase. Following 4BAH2 addition, most vesicles appear to retain their initial size but they are predominantly organized into large aggregates. Note the large size of the aggregates as indicated by the scale bar. Atomic force microscopy (AFM) further confirmed vesicle aggregation. Note the image size as indicated by the white scale bars. (H) Bare, hydrophilic SiOₓ substrate. (I) POPC vesicles fused onto the SiOₓ substrate and formed a supported bilayer. (J) 4BAH2 was first added to the vesicle solution and then the vesicle aggregates fused onto the SiOₓ substrate. No supported bilayer was formed and there is instead significant adsorption of aggregated vesicles on the SiOₓ substrate. Note the extremely large size of the vesicle aggregates as indicated by the line scans in the bottom panels, as marked by the red and green arrows.

FIG. 13. Disruption of 4BAH2's amphipathic nature abrogates vesicle aggregation. (A) Helix net diagrams (depicted in the N-terminal to C-terminal from bottom to top) of amino acids 40-62 of NS4B, which comprise 4BAH2, wherein the point mutations introduced into each of the three 4BAH2 mutants are indicated in red. Similar to FIG. 12C, the POPC lipid vesicle size distribution was measured by DLS in the (B) absence or (C) presence of 4BAH2, or in the presence of mutant versions of 4BAH2 harboring (D) two point mutations, 4BAH2 (M2), (E) three point mutations, 4BAH2 (M3), or (F) four point mutations, 4BAH2 (M4). Note the x-axis scale is separated into two linear size ranges in order to directly compare the dramatic increase in the average vesicle size distribution upon addition of the wild type, but not mutant, 4BAH2 peptides. (G) Far-UV circular dichroism (CD) spectra of the 4BAH2 mutants of FIG. 13A.

FIG. 14. Identification of small molecule inhibitors by a fluorescence-based, high throughput assay based on 4BAH2-induced vesicle aggregation. (A) Schematic of the imaging-based method used to quantitatively analyze the high throughput screening measurements of 4BAH2-induced aggregation of lipid vesicles containing a fluorescent lipid probe, N-(7-nitrobenz-2-oxa-1,3-diazol-4-yl)-1,2-dihexadecanoyl-sn-glycero-3-phosphoethanolamine, triethylammonium salt (Texas Red-DHPE), prepared in a 99.5:0.5 molar ratio (POPC:Texas Red-DHPE). Inhibition of this mode of vesicle aggregation by small molecule compounds was monitored. The assay was performed in a 384 well format and analyzed by automated high-content microscopic imaging coupled with pattern recognition software trained to quantify the fluorescence contained within granule size aggregates of vesicles in order to identify small molecule inhibitor candidates. (B) The 4BAH2-induced aggregation of lipid vesicles could be readily visualized by automated fluorescence microcopy (10×). Although individual vesicles (POPC) are too small to be visualized with a fluorescence microscope, aggregated vesicles could be visualized following the addition of 4BAH2 (POPC+4BAH2). The additional two panels are examples of selected positive hits with drugs that prevented vesicle aggregation by inhibiting 4BAH2's mode of action. (C) The presence or absence of aggregates was analyzed in a high throughput fashion using pattern recognition software based on total granule area of vesicle aggregation induced by 4BAH2. (D) DLS assay on candidate molecules identified to be positive in the high throughput screening assay (and controls). Selected molecules, presented in FIG. 14C, that scored positive or negative in the assay were further analyzed by DLS. The title POPC indicates solely POPC lipid vesicles. DMSO indicates the addition of DMSO to the POPC vesicle solution; DMSO was added to all samples except the pure vesicle solution. 4BAH2 was added to all other DLS measurements except POPC and POPC+DMSO, including all candidate compound screenings. Compounds CZ, H8, and H10 were included as negative controls.

FIG. 15. Inhibition of HCV genome replication by selected identified small molecule inhibitors of 4BAH2 function, which correlates with genotype specificity observed in the DLS assay. Huh7.5 cells were electroporated with a subgenomic genotype 1b replicon RNA (Bart79ILuc) ((A) and (C)), or full-length genotype 2a HCV RNA (J6/JFH Luc) ((B) and (D)), and then treated daily with fresh medium containing the indicated amounts of compounds (compound C4 for (A) and (B); compound A2 for (C) and (D)). Both constructs harbor luciferase reporter genes, and luciferase assays were performed at 72 hr after the beginning of the treatment to assess viral genome replication, in parallel with Alamar Blue-based viability assays. The average of at least three independent experiments with four replicates is shown. The significant size changes measured by DLS following the addition of 4BAH2 reflect vesicle aggregation, and the genotype specificity of the compounds' inhibition of 4BAH2-induced vesicle aggregation correlates with the observed genotype specificity of the compounds' inhibition of viral genome replication. After measuring the size distribution of (E) pure POPC vesicles, we added (F) genotype 1b 4BAH2 or (G) genotype 2a 4BAH2 to solutions of pure vesicles. As we expected, 4BAH2 of both genotypes 1b and 2a induced a similar aggregation of vesicles. Further, we monitored the interaction of each genotype 4BAH2 peptide with two different candidate compounds, A2 and C4. Both compounds inhibited genotype 1b 4BAH2-mediated vesicle aggregation (G, I). However, A2 had no significant effect on the vesicle aggregation induced by genotype 2a 4BAH2 (H), while C4 did (J). Parallel genotype-specific effects on viral genome replication were observed (A-D). Note the x-axis scale is separated into two linear size ranges in order to directly compare the dramatic increase in the average vesicle size distribution.

FIG. 16. Model of 4BAH2 self-oligomerization and induction of lipid vesicle aggregation. A model representation of how 4BAH2 induces the aggregation of lipid vesicles. (A) Red and blue represent the hydrophilic and hydrophobic portions, respectively, of the amphipathic alpha helical 4BAH2 peptide derived from NS4B. Pale red/pink spheres represent the POPC vesicles with electric field representation. Light blue dots represent the solvent. In solution, 4BAH2 peptides aggregate via hydrophobic mismatch to reduce the number of unfavorable hydrophilic-hydrophobic interactions as shown in the top view. The side view is also presented. (B) Upon adding 4BAH2 peptides to a vesicle solution, hydrophilic parts of the peptides interact with the vesicles and hydrophobic portions of 4BAH2 interact with each other to promote aggregation of peptide-vesicle complexes, as experimentally shown in FIG. 14. (C) A model representation of how selected drug candidates inhibit 4BAH2-mediated lipid vesicle aggregation. Here we propose two different possible mechanisms, in which one derives by hydrophilic dissociation, and the other involves hydrophobic dissociation. In the case of hydrophilic dissociation, the candidate drug (green/yellow molecules) interacts with the hydrophilic portion of the peptide so that it inhibits 4BAH2's association with the charged lipid head groups of the bilayer, therefore abrogating vesicle aggregation. By contrast, in the case of hydrophobic dissociation, different classes of drugs (schematized in yellow) can interact with the hydrophobic face of 4BAH2 so that they prevent peptide oligomerization via these interfaces, leading to abrogation of vesicle aggregation but not the association of 4BAH2 with the bilayer.

FIG. 17. C4 inhibits aggregation, but not membrane association, of 4BAH2, while A2 inhibits membrane association, but not aggregation, of 4BAH2, as determined by AFM and QCM-D. AFM images of C4 inhibiting oligomerization of 4BAH2 peptide on a bare, hydrophilic SiOₓ substrate (A to D, top panels). The scan size is 5 μm×5 μm. Linescans at the level of two different arrowheads (red and green) are indicated in their respective images (A-D, middle panels). (A) Bare, hydrophilic SiOₓ substrate. (B) 4BAH2 aggregates on SiOₓ substrate following adsorption. The length of the aggregated peptides can exceed 600 nm. (C) Drug candidate C4 interacts with 4BAH2 to inhibit peptide aggregation. (D) In marked contrast, drug candidate A2 does not interact with 4BAH2 and thus does not prevent peptide aggregation. Magnified views of the indicated boxed areas of the top panels are shown in the bottom panels of 7A to D. The QCM-D technique monitors 4BAH2's interaction with a SiOₓ-supported POPC bilayer platform, revealing that 4BAH2 is necessary and sufficient for NS4B's membrane association on a POPC lipid bilayer. (E) We first created a POPC bilayer on a SiOₓ-coated quartz crystal (indicated by arrow 1), at which point lipid vesicles were added followed by their subsequent fusion to create a lipid bilayer on the oscillating quartz crystal (recognizable by the frequency change of −25 Hz that is characteristic for bilayer formation. Genotype 1b 4BAH2 peptide was then injected (arrow 2). The resulting frequency change upon addition of peptides (an additional −25 Hz decrease) demonstrates deposition and binding of a large mass to the membrane bilayer (1 Hz˜17.7 ng/cm²). Corresponding energy dissipation changes of ˜5×10⁻⁶ indicates that the peptide associates with the bilayer in an oligomerized state. (F) Similar to FIG. 17E, except that genotype 2a 4BAH2 peptide was used. The results indicate that membrane association of this genotype's 4BAH2 is similar to that of genotype 1b. (G) QCM-D monitoring of a selected molecule, A2, identified to be positive for inhibition in the vesicle aggregation assay. After forming a POPC bilayer on a SiOₓ-coated quartz crystal (after arrow 1), compound A2 was injected together with genotype 1b 4BAH2 (arrow 2) in order to follow any effects on the interactions observed in FIG. 17E. The lower change in frequency (compared to 18E) indicates an ˜83 percent reduction in binding mass of 4BAH2 to the bilayer due to inhibition by A2. (H) The most interesting results are seen with genotype 2a 4BAH2's interaction with the bilayer in the presence of A2. Namely, this compound does not inhibit 4BAH2 membrane association (indicated by no inhibition of the large decrease in frequency change following addition of 4BAH2 (arrow 2)), highlighting the genotype specificity of A2. Similar experiments were performed to study C4's interaction with 4BAH2. In contrast to A2, C4 exhibited no effect on the membrane association of 4BAH2 of either (I) genotype 1b, or (J) genotype 2a.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a peptide” includes a plurality of such peptides and reference to “the inhibitor” includes reference to one or more inhibitors and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention.

Further, the dates of publication provided may be different from the actual publication dates which may need to be independently confirmed.

### DETAILED DESCRIPTION OF THE EMBODIMENTS

Screening methods are provided for identifying pharmacologic inhibitors of HCV amphipathic helix (AH) function, which inhibitors are useful in the prevention and treatment of HCV infection. The methods of the invention are based on the unexpected discovery that the presence of an AH, e.g. an AH of an HCV polypeptide, causes an increase in the apparent diameter of the vesicles to which the AH is added. The methods of the invention provide for addition of AH peptides to lipid vesicles, for example in a high-throughput format; which addition may be performed in the absence or presence of a candidate pharmacologic agent. The change in vesicle size is measured, and compared to control samples. An increase in vesicle size is indicative of AH function being present; and a lack of increase is indicative that the AH function is absent or has been inhibited by a test agent.

In some embodiments the increase in vesicle size is monitored by a change in dynamic light scattering (DLS). (For a review of DLS methods, see Pencer and Hallett (2003) Langmuir 19, 7488-7497; and Lesieur et al. (1991) Anal Biochem. 192(2):334-43, each herein specifically incorporated by reference).

In other embodiments the change in vesicle size is monitored by visual inspection, by plate reader, by determination of light transmission or altered fluorescence properties, and the like. For example, screening may utilize fluorescence dequenching assays wherein a self-quenching fluorescent lipid is incorporated into a population of vesicles and mixed with unlabelled vesicles in the presence of AH peptide in the absence or presence of a candidate inhibitor agent. Upon vesicle fusion, the quenched fluorescent lipid distributes over a greater surface area with loss of self quenching and an increase in the emitted fluorescence. Alternatively, labeling of two vesicle populations with one of two appropriate partner molecules, can allow for fluorescence resonance energy transfer (FRET) when the two partners are brought into close enough proximity as a result of AH-induced vesicle aggregation. When performed in the presence of a candidate pharmacologic inhibitor, FRET inhibition is monitored to identify a hit molecule. In other embodiments, inhibition of AH-induced vesicle aggregation in the presence of a candidate inhibitor agent can be monitored by observing the absence of the gross aggregates using high throughput microscopy. Detection of the aggregates can be facilitated by using fluorescently-labeled lipid vesicles and a fluorescence microscope.

### DEFINITIONS

Amino acid alpha helices can be identified by examination of structural data, such as crystal structure data, or by use of secondary structure prediction analysis of primary sequence data, or some combination of both. Next, a “helix wheel” program can be used to plot or visualize the alpha helix. In such helix wheel plots, adjacent amino acids are plotted around a circle, with a ˜100 degree angle between them. Any method or program that allows for the relative orientation of the amino acid side chains in the helix, with respect to one another, to be determined can be used. Next, such plots can be analyzed, such as by inspection or other means, to determine if the helix under examination has the following properties: (a) a hydrophobic face or surface, and (b) a hydrophilic surface that can include negatively (such as the acidic amino acids glutamate, aspartate) or positively charged amino acids (usually in the form of the basic amino acids lysine (K), arginine (R), or histidine (H)), including an orientation where the latter usually flank the hydrophobic face and are oriented in the same general direction as the hydrophobic face.

Mathematical/automated algorithmic processes of identifying amphipathic helices have been described, such as Amphipaseek (Sapay et al. BMC Bioinformatics 2006) or WHEEL, HELNET, COMBO, COMNET, CONSESUS (Jones et al. J of Lipid Research 1992). These methods sometimes use mathematical/algorithmic methods of identifying polypeptides that, if helical, would possess hydrophobic faces or surfaces, such as using the method called the hydrophobic moment (Eisenberg et al. PNAS 1984). These methods also sometimes use mathematical/algorithmic methods of secondary structure prediction.

These programs identify regions of a polypeptide(s) that form potential or actual alpha helices with potential or actual hydrophobic faces or regions and may result in “helix wheel” plots or other structural plots, or in the identification of what are known as Class A amphipathic helices (Segrest et al. Proteins 1990), which are amphipathic helices with positive charged residues at the hydrophobic-hydrophilic interface and negatively charged residues on the hydrophilic face. AHs can also be identified by displaying the amino acid sequence in a simple helix net diagram (such as in Elazar et al. J. Virol. 2003).

Examples of HCV AH peptides include those found in NS4B, including, without limitation, NS4B AH1; NS4B AH2; and the NS5A AH. Peptides of interest for assays include, without limitation, a peptide of about 8 amino acids, of about 10 amino acids, of about 12 amino acids, of about 14 amino acids, of about 16 amino acids, of about 18 amino acids, of about 20 amino acids, of about 22 amino acids, of about 24 amino acids, of about 26 amino acids, of about 28 amino acids, of about 30 amino acids, or more; and having the residues conserved across genotypes or the residues sufficient for AH function. Sequences for NS4B AH1 and NS5A AH include those outlined in PCT publication WO 2002/089731 and US2008/0125367 (incorporated herein by reference) applications.

Sequences for 4BAH2 include: amino acids 43 to 65 of NS4B (genotype 1b): (SEQ ID NO:16) WRTLEAFWAKHMWNFISGIQYLA, or amino acids 38 to 67 (SEQ ID NO:17) VVESKWRTLEAFWAKHMWNFISGIQYLAGL, and smaller versions within these boundaries, as well as the corresponding sequences in other HCV genotypes and isolates readily available in public databases, for example genotype 1a (AF009606) (SEQ ID NO:1) AVQTNWQKLEVFWAKHMWNFISGIQYLAGL; genotype 1b (as found in Elazar et al. 2003 J. Virol.) (SEQ ID NO:2) WESKWRTLEAFWAKHMWNFISGIQYLAGL; genotype 2a (AB047639) (SEQ ID NO:3) AMQASWPKVEQFWARHMWNFISGIQYLAGL; genotype 3a (AF046866) (SEQ ID NO:4) IVATNWQKLEAFWHKHMWNFVSGIQYLAGL; genotype 4a (DQ418782) (SEQ ID NO:5) VIQSNFAKLEQFWAKHMWNFISGIQYLAGL; genotype 5a (AF064490) (SEQ ID NO:6) AATSMWNRAEQFWAKHMWNFVSGIQYLAGL; genotype 6a (AY859526) (SEQ ID NO:7) AVHSAWPRMEEFWRKHMWNFVSGIQYLAGL; (SEQ ID NO:8) VVESKWRTLEAFWAKHMWNFISGVQYLAGL; (SEQ ID NO:9) VVESKWRTLETFWAKHMWNFISGIQYLAGL; (SEQ ID NO:10 VVESKWRTLETFWAKHMWNFISGIQYLAGL; (SEQ ID NO:11) VVESKWRSLEAFWAKHMWNFISGIQYLAGL; (SEQ ID NO:12) VVESKWRSLEAFWAKHMWNFISGIQYLAGL; (SEQ ID NO:13) VVESKWRSLEAFWAKHMWNFISGIQYLAGL; (SEQ ID NO:14) VVESKWRTLETFWAKHMWNFISGIQYLAGL; (SEQ ID NO:15) VVESKWRALEAFWAKHMWNFISGIQYLAGL, and including fragments and derivatives thereof. In some embodiments, the peptide of SEQ ID NO:16 is preferred. The peptide may have a carboxyl group at the C-terminus, or may be amidated at the C terminus.

In some embodiments of the invention, 4BAH2 peptides, e.g. peptides consisting of, or comprising the sequences set forth above, of fragments or derivatives thereof, are useful, for example, in aggregation of vesicles. The ordinarily skilled artisan can readily generate variants of the AH peptide amino acid sequences described herein. For example, such substitutions can be made so that they are spaced at intervals along the predicted α-helix such that an α-helical structure with a hydrophobic face and a hydrophilic face is maintained. Thus AH peptide variants that retain activity in membrane aggregation that have, for example, conservative amino acid substitutions relative to a naturally-occurring AH peptide amino acid sequence so as to result in replacement of amino acid residues of an AH peptide with residues that provide for similar charge, polarity, and retain the α-helical structure can be readily generated.

It is appreciated that certain amino acid substitutions can result in peptides can disrupt the formation of the helix; however, the nature of these substitutions is already understood by those of ordinary skill and can be avoided, or purposefully used, as desired. Insertion of, for example, disruptive proline residues, can be undesirable. Thus, it is well within ordinary skill to substitute one or more amino acids in these sequences to obtain AH peptides that retain the desired activity in disrupting viral envelopes.

AH peptides can have residues linked by native amide bonds or by non-native bonds. Reference to “peptide” herein is meant to encompass both a polymer of amino acids linked by a native amide bonds or non-native amide bonds.

It should be understood that as used throughout, and unless specifically indicated otherwise, the term “amino acid” is used herein in its broadest sense, and includes naturally occurring amino acids as well as non-naturally occurring amino acids, including amino acid analogs and derivatives. The latter includes molecules containing an amino acid moiety. One skilled in the art will recognize, in view of this broad definition, that reference herein to an amino acid includes, for example, naturally occurring proteogenic L-amino acids; D-amino acids; chemically modified amino acids such as amino acid analogs and derivatives; naturally occurring nonproteogenic amino acids such as norleucine, p-alanine, ornithine, etc.; and chemically synthesized compounds having properties known in the art to be characteristic of amino acids. As used herein, the term “proteogenic” indicates that the amino acid can be incorporated into a peptide, polypeptide, or protein in a cell through a metabolic pathway.

The incorporation of non-natural amino acids, including synthetic non-native anlino acids, substituted amino acids, or one or more D-amino acids into the present AH peptides can provide for, for example, increased stability in vitro or in vivo (e.g., D-amino acid-containing peptides as compared to L-amino acid-containing peptides). For example, AH peptides incorporating 1, 2, 3, 4, 5, 6, 7, 8, 9, 10 or more D-amino acids can be particularly useful when greater stability (e.g., in an in vivo setting) is desired or required. For example, D-amino acid-containing peptides can be provided that are resistant to peptidases and proteases, thereby providing improved bioavailability of the molecule, and prolonged lifetimes in vivo and in vitro when such properties are desirable. Moreover, D-amino acid-containing peptides are not efficiently processed for major histocompatibility complex class 11-restricted presentation to T helper cells, and are therefore less likely to induce humoral immune responses in the whole organism than purely L-amino acid-containing peptides.

Selection of amino acid residues for use in an AH peptide, particularly one based on a naturally-occurring amphipathic, α-helical amino acid sequence, can take into consideration the hydropathic index of the amino acid present in the reference sequence and the hydropathic index of the amino acid residue proposed for substitution. The importance of the hydropathic amino acid index in conferring interactive biological action on a protein has been discussed by Kyte and Doolittle (1982, J. Mol. Biol., 157: 105-132). It is accepted that the relative hydropathic character of amino acids contributes to the secondary structure of the resultant protein. This, in turn, affects the interaction of the protein with other molecules. Amino acid substitutions in the AH peptides can be based on the relative similarity of the amino acid side-chain substituents, for example, their hydrophobicity, hydrophilicity, charge, size, etc.

Exemplary substitutions that take various of the foregoing characteristics into consideration in order to produce conservative amino acid changes resulting in AH peptides having changes that do not substantially affect activity in disrupting viral envelopes can be selected from other members of the class to which the naturally occurring amino acid belongs. Amino acids can be divided into the following four groups: (1) acidic amino acids; (2) basic amino acids; (3) neutral polar amino acids; and (4) neutral non-polar amino acids. Representative amino acids within these various groups include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, cystine, tyrosine, asparagine, and glutamine; and (4) neutral non-polar amino acids such as alanine, leucine, isoleucine, valine, proline, phenylalanine, tryptophan, and methionine.

The AH peptides can be provided in the context of the nonstructural protein or fragment thereof or can be provided in the form of a fusion protein between an AH peptide and a heterologous polypeptide. For example, the AH peptide can be provided as a fusion protein that contains a detectable label, such as a fluorescent polypeptide (e.g., green fluorescent protein) or an immunodetectable label (e.g., FLAG, which can be exploited to facilitate isolation by immunoisolation techniques). In other examples of AH peptide-containing fusion proteins, the heterologous polypeptide can be a virucidal peptide, a lipid binding protein (e.g., to facilitate clearance of lipids that may be by-products of disruption of viral envelopes, a polypeptide that enhances serum half-life (e.g., by increasing the size of the molecule, such as a PEGylated polypeptide), an antibody or antigen binding fragment thereof; or a polypeptide that facilitates recombinant production and/or isolation. Such AH peptide fusion proteins may include a spacer between the AH peptide amino acid sequence and the amino acid sequence of the heterologous polypeptide (e.g., to facilitate presentation of the amphipathic α-helix to viral envelopes).

For use in the assay methods of the invention, the peptide may be detectably labeled, e.g., is directly detectably labeled. Suitable detectable labels include, e.g., radiolabels; enzymes that act on a substrate to yield a colored, luminescent, or fluorescent product; fluorescent proteins (a green fluorescent protein, a yellow fluorescent protein, a red fluorescent protein, etc.); a fluorophore (e.g., fluorescein, rhodamine, tetramethylrhodamine, eosin, erythrosin, coumarin, methyl-coumarins, pyrene, Malacite green, stilbene, Lucifer Yellow, Cascade Blue™, Texas Red, IAEDANS, EDANS, BODIPY FL, LC Red 640, Cy 5, Cy 5.5, LC Red 705 and Oregon green); and the like.

As used herein the term “isolated,” when used in the context of an isolated compound, refers to a compound of interest that is in an environment different from that in which the compound naturally occurs. “Isolated” is meant to include compounds that are within samples that are substantially enriched for the compound of interest and/or in which the compound of interest is partially or substantially purified. For example, an isolated peptide of the invention is at least 60%, by weight, free from the proteins and naturally-occurring organic molecules with which it is naturally associated or, in the context of synthetic peptides, at least 60% by weight free of synthetic peptides of different sequence and intermediates. Preferably, the preparation is at least 75%, more preferably at least 90%, and most preferably at least 99%, by weight, peptide. An isolated peptide as described herein may be obtained, for example, by chemically synthesizing the protein or peptide, or by expression of a recombinant nucleic acid encoding a peptide of interest, with chemical synthesis likely being preferred. Purity can be measured by any appropriate method, e.g., column chromatography, mass spectrometry, HPLC analysis, and the like.

The terms “active agent,” “antagonist”, “inhibitor”, “drug” and “pharmacologically active agent” are used interchangeably herein to refer to a chemical material or compound which, when administered to an organism (human or animal) induces a desired pharmacologic and/or physiologic effect by local and/or systemic action.

As used herein, the terms “treatment,” “treating,” and the like, refer to obtaining a desired pharmacologic and/or physiologic effect, such as reduction of viral titer. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease (as in liver fibrosis that can result in the context of chronic HCV infection); (b) inhibiting the disease, i.e., arresting its development; and (c) relieving the disease, i.e., causing regression of the disease (e.g., reduction in viral titers).

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations.

As used herein, the terms “determining,” “measuring,” “assessing,” and “assaying” are used interchangeably and include both quantitative and qualitative determinations.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and native leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like.

A “therapeutically effective amount” or “efficacious amount” means the amount of a compound that, when administered to a mammal or other subject for treating a disease, condition, or disorder, is sufficient to effect such treatment for the disease, condition, or disorder. The “therapeutically effective amount” will vary depending on the compound, the disease and its severity and the age, weight, etc., of the subject to be treated.

The term “unit dosage form,” as used herein, refers to physically discrete units suitable as unitary dosages for human and animal subjects, each unit containing a predetermined quantity of a compound calculated in an amount sufficient to produce the desired effect in association with a pharmaceutically acceptable diluent, carrier or vehicle. The specifications for unit dosage forms depend on the particular compound employed and the effect to be achieved, and the pharmacodynamics associated with each compound in the host.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

### METHODS OF THE INVENTION

It has now been found that an amphipathic helix peptide from an HCV polypeptide, for example the NS5A, NS4B, NS5B AH peptides, function to increase the apparent size of lipid vesicles when the peptides are added to a suspension of the vesicles. For purposes of the assay methods the AH may be provided as an isolated peptide, or in the context of a larger peptide, e.g. the intact HCV NS4B, NS5A, NS5B, etc. polypeptide or fragments thereof. The peptide may also be utilized as a fusion protein that contains a label, such as green fluorescent protein, or as a labeled peptide, as described above. For purposes of the assay the AH peptide is suspended in any suitable buffer, and will be added to a suspension of lipid vesicles in an amount of from about 1 attomole to about 1 femtomole, from about 1 femtomole to about 1 picomole, from about 1 picomole to about 1 nanomole, from about 1 nanomole to about 50 nanomoles, from about 50 nanomoles to about 100 nanomoles, from about 100 nanomoles to about 500 nanomoles, from about 500 nanomoles to about 1 μmole, from about 1 μmole to about 50 μmoles, from about 50 μmoles to about 100 μmoles, from about 100 μmoles to about 500 μmoles, from about 500 μmoles to about 1 mmole, from about 1 mmole to about 50 mmole, from about 50 mmole to about 100 mmole, or greater than 100 mmole.

Any convenient format may be used for the assay, e.g. wells, plates, flasks, etc., preferably a high throughput format, such as multi-well plates. Typically a suspension of lipid vesicles is placed in wells, where varying concentrations may be used. Lipid vesicles may be formed by methods known in the art, e.g. sonication, extrusion, etc. The composition of lipids may be varied according to the desired assay, but will typically include comprise a population of substantially unilamellar vesicles bounded by a lipid bilayers, which lipid bilayers may comprise one or a plurality of different amphipathic molecules, i.e. lipids, and may further comprise polypeptides, cholesterol, etc. as known in the art. For example, vesicles of interest may be derived from cellular internal or external membranes, e.g. microsomes, erythrocyte membranes, etc. Vesicles of interest may be substantially homogeneous in size, or may provide for a variable population, with the proviso that the population permits detection of a size change by the addition of an AH peptide. Vesicle sizes may range from about 25 nm in diameter, about 100 nm in diameter, about 200 nm in diameter, about 400 nm in diameter, about 500 nm in diameter, about 1 μm in diameter, to not more than about 10 μm in diameter.

A mixture of lipid molecules may provide different functional groups on the hydrophilic exposed surface. For example, some hydrophilic head groups may have functional surface groups, for example, biotin, amines, cyano, carboxylic acids, isothiocyanates, thiols, disulfides, α-halocarbonyl compounds, α,β-unsaturated carbonyl compounds and alkyl hydrazines for attachment of moieties for detection of size changes, etc.

Lipids of interest include fatty acids, neutral fats such as triacylglycerols, fatty acid esters and soaps, long chain (fatty) alcohols and waxes, sphingoids and other long chain bases, glycolipids, sphingolipids, carotenes, polyprenols, sterols, and the like, as well as terpenes and isoprenoids. For example, molecules such as diacetylene phospholipids may find use. Specific lipids of interest include various phosphocholines, e.g. 1-palmitoyl-2-oleoyl-sn-glycero-3-phosphocholine, 1,2-Dioleoyl-sn-glycero-3-phosphocholine (DOPC) and 1-stearoyl-2-oleoyl-sn-glycero-3-phosphocholine (SOPC), etc.

In one embodiment of the invention, an AH peptide, e.g. an NS4BAH2 peptide is combined with a candidate agent, to which small unilamellar lipid vesicles of POPC, e.g. prepared by an extrusion method (see, for example, Cho et al. J. Virology, 81, 2007, 6682) through 0.03 μm membranes. The change in vesicle aggregation may be monitored by visual inspection, or a dynamic light scattering reader. A low throughput assay may utilize, for example, vials, plates, etc., while a high throughput assay will generally utilize multi-well plates, and compounds will be tested at multiple dilutions and in replica.

A test agent of interest is added to the reaction mixture with the AH peptide, usually in different concentrations, and the effect of the agent on vesicle size is determined by DLS, visual inspection, fluorescence, etc., where an inhibitor of AH function will inhibit the increase in apparent vesicle size.

Test agents of interest inhibit AH peptide function by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, or at least about 90%, or more, compared to the function in the absence of the test agent.

A variety of different test agents may be screened using a subject method. Candidate agents encompass numerous chemical classes, e.g., small organic compounds having a molecular weight of more than 50 daltons and less than about 10,000 daltons, less than about 5,000 daltons, or less than about 2,500 daltons. Test agents can comprise functional groups necessary for structural interaction with proteins, e.g., hydrogen bonding, and can include at least an amine, carbonyl, hydroxyl or carboxyl group, or at least two of the functional chemical groups. The test agents can comprise cyclical carbon or heterocyclic structures and/or aromatic or polyaromatic structures substituted with one or more of the above functional groups. Test agents are also found among biomolecules including peptides, saccharides, fatty acids, steroids, purines, pyrimidines, derivatives, structural analogs or combinations thereof.

Test agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Moreover, screening may be directed to known pharmacologically active compounds and chemical analogs thereof, or to new agents with unknown properties such as those created through rational drug design.

In some embodiments, test agents are synthetic compounds. A number of techniques are available for the random and directed synthesis of a wide variety of organic compounds and biomolecules, including expression of randomized oligonucleotides. See for example WO 94/24314, hereby expressly incorporated by reference, which discusses methods for generating new compounds, including random chemistry methods as well as enzymatic methods.

In another embodiment, the test agents are provided as libraries of natural compounds in the form of bacterial, fungal, plant and animal extracts that are available or readily produced. Additionally, natural or synthetically produced libraries and compounds are readily modified through conventional chemical, physical and biochemical means. Known pharmacological agents may be subjected to directed or random chemical modifications, including enzymatic modifications, to produce structural analogs.

In some embodiments, the test agents are organic moieties. In this embodiment, as is generally described in WO 94/243 14, test agents are synthesized from a series of substrates that can be chemically modified. “Chemically modified” herein includes traditional chemical reactions as well as enzymatic reactions. These substrates generally include, but are not limited to, alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracyclines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Chemical (including enzymatic) reactions may be done on the moieties to form new substrates or candidate agents which can then be tested using the present invention.

As used herein, the term “determining” refers to both quantitative and qualitative determinations and as such, the term “determining” is used interchangeably herein with “assaying,” “measuring,” and the like.

In some embodiments test agents are assessed for any cytotoxic activity it may exhibit toward a living eukaryotic cell, using well-known assays, such as trypan blue dye exclusion, an MTT (3-(4,5-dimethylthiazol-2-yl)-2,5-diphenyl-2H-tetrazolium bromide) assay, and the like. Agents that do not exhibit significant cytotoxic activity are considered candidate agents.

A variety of other reagents may be included in the screening assay. These include reagents like salts, neutral proteins, e.g. albumin, detergents, etc., including agents that are used to facilitate optimal binding activity and/or reduce non-specific or background activity. Reagents that improve the efficiency of the assay, such as protease inhibitors, nuclease inhibitors, anti-microbial agents, etc. may be used. The components of the assay mixture are added in any order that provides for the requisite activity. Incubations are performed at any suitable temperature, typically between 4° C. and 40° C. Incubation periods are selected for optimum activity, but may also be optimized to facilitate rapid high-throughput screening. In some embodiments, between 0.1 hour and 1 hour, between 1 hour and 2 hours, or between 2 hours and 4 hours, will be sufficient.

Assays of the invention include controls, where suitable controls include a sample (e.g., a sample comprising an AH peptide in the absence of the test agent). Generally a plurality of assay mixtures is run in parallel with different agent concentrations to obtain a differential response to the various concentrations. Typically, one of these concentrations serves as a negative control, i.e. at zero concentration or below the level of detection.

In some embodiments, a test agent that inhibits AH peptide function is further tested for its ability to inhibit HCV replication in a cell-based assay. In these embodiments, a test agent of interest is contacted with a mammalian cell that harbors all or part of an HCV genome; and the effect, if any, of the test agent on HCV replication is determined. Suitable cells include mammalian liver cells that are permissive for HCV replication, e.g., an immortalized human hepatocyte cell line that is permissive for HCV. For example, a suitable mammalian cell is Huh7 hepatocyte or a subclone of Huh7 hepatocyte, e.g., Huh-7.5. Suitable cell lines are described in, e.g., Blight et al. (2002) J. Virol. 76:13001; Zhang et al. (2004) J. Virol. 78:1448; and Einav et al. (2008) Nat. Biotech 26(9): 1019-1027. In some embodiments, the HCV genome in the cell comprises a reporter, e.g., a nucleotide sequence encoding luciferase, a fluorescent protein, or other protein that provides a detectable signal; and determining the effect, if any, of the test agent on HCV replication is achieved by detection of a signal from the reporter.

Thus, in some embodiments, a test agent of interest inhibits HCV replication by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, or at least about 90%, or more, compared to the level of HCV replication in the absence of the test agent.

### Pyrazine-2-carboxamide

In some embodiments of the invention, a pyrazine-2-carboxamide analog is utilized for the inhibition of viral infection, e.g. in the treatment or prevention of infection, in competitive assays; in testing with combination therapies, and the like. For example, the pyrazine-2-carboxamide analogs are administered alone or in combination with other active agents to a patient suffering from an HCV infection, in a dose and for a period of time sufficient to reduce the patient population of pathogenic viruses or reduce plaque formation. Alternatively, a pharmaceutical composition comprising a pyrazine-2-carboxamidepyrazine-2-carboxamide analog of the invention is administered as a protective agent to a normal individual.

Formulations of a pyrazine-2-carboxamide analog of the invention are administered to a host suffering from an ongoing viral infection or who faces exposure to a viral infection. Administration may be topical, localized or systemic, depending on the specific patient needs. Generally the dosage will be sufficient to decrease the viral population by at least about 50%, usually by at least 1 log, and may be by 2 or more logs. The compounds of the present invention are administered at a dosage that reduces the pathogen replication while minimizing any side-effects. It is contemplated that the composition will be obtained and used under the guidance of a physician for in vivo use.

Pyrazine-2-carboxamide analogs of the invention are also useful for in vitro formulations to inactivate viruses. For example, a pyrazine-2-carboxamide analog of the invention may be added to animal and/or human food preparations, or to blood products intended for transfusion to reduce the risk of consequent viral infection. A pyrazine-2-carboxamide analog of the invention may be included as an additive for in vitro cultures of cells, to prevent the infection in tissue culture.

The susceptibility of a particular virus to inhibition by a pyrazine-2-carboxamide analog of the invention may be determined by in vitro testing, as detailed in the experimental section. Typically a culture comprising a test virus is combined with a pyrazine-2-carboxamide analog of the invention at varying concentrations for a period of time sufficient to allow the agent to act, usually ranging from about one hour to one day. The viral replication is then measured.

Various methods for administration may be employed. The formulation may be given orally, or may be injected intravascularly, subcutaneously, peritoneally, by aerosol, opthalmically, intra-bladder, topically, etc. The dosage of the therapeutic formulation will vary widely, depending on the specific pyrazine-2-carboxamide analog of the invention to be administered, the nature of the disease, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered once or several times daily, semi-weekly, etc. to maintain an effective dosage level. In many cases, oral administration will require a higher dose than if administered intravenously.

Pyrazine-2-carboxamide analogs of the invention have the structure I:

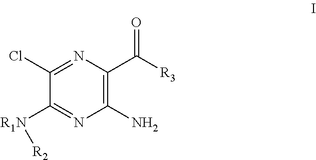

where R₁ and R₂ are independently selected from hydrogen; a lower C1-C6 alkyl, which may be branched or unbranched; or a benzyl; and

R₃ is NHR₄ or OR₄, where R₄ is selected from hydrogen, a lower alkyl, and CHR₅, where R₅ is selected from thiophene, isoxazole, thiazoles, pyridine, thiadiazole, benzene, cyclohexane, piperidine, and pyrrolidine, any of which is optionally substituted with one or more substituents, including lower alkyl, halogen, e.g. Br, Cl, F, I; carboxylic acid moiety; and the like.

In some embodiments the pyrazine-2-carboxamide analog has the formula of structure II:

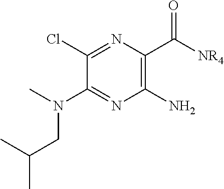

where R₄ is as defined above.

A pyrazine-2-carboxamide analog of interest for use in the methods of the invention is 3-amino-N-carbamimidoyl-6-chloro-5-(isobutyl(methyl)amino)pyrazine-2-carboxamide which is also referenced in the Examples herein as “C4”.

Specific analogs of interest also include those set forth in Table 1, including methyl 3-amino-6-chloro-5-(isobutyl(methyl)amino)pyrazine-2-carboxylate; 3-amino-6-chloro-5-(isobutyl(methyl)amino)pyrazine-2-carboxylic acid; 3-amino-6-chloro-5-(isobutyl(methyl)amino)-N-(thiophen-3-ylmethyl)pyrazine-2-carboxamide hydrochloride; 3-amino-6-chloro-5-(isobutyl(methyl)amino)-N-(thiazol-2-ylmethyl)pyrazine-2-carboxamide hydrochloride; 3-amino-6-chloro-5-(isobutyl(methyl)amino)-N-(thiophen-2-ylmethyl)pyrazine-2-carboxamide 2,2,2-trifluoroacetate; 3-amino-6-chloro-5-(isobutyl(methyl)amino)-N-(isoxazol-3-yl)pyrazine-2-carboxamide hydrochloride; 3-amino-6-chloro-5-(isobutyl(methyl)amino)-N-(pyridin-2-yl)pyrazine-2-carboxamide hydrochloride; 3-amino-6-chloro-5-(isobutyl(methyl)amino)-N-(pyridin-3-yl)pyrazine-2-carboxamide hydrochloride; 3-amino-6-chloro-5-(isobutyl(methyl)amino)-N-(pyridin-4-yl)pyrazine-2-carboxamide hydrochloride; 3-amino-6-chloro-5-(isobutyl(methyl)amino)-N-(pyridin-2-ylmethyl)pyrazine-2-carboxamide hydrochloride; 3-amino-6-chloro-5-(isobutyl(methyl)amino)-N-(1,3,4-thiadiazol-2-yl)pyrazine-2-carboxamide hydrochloride; 3-amino-6-chloro-5-(isobutyl(methyl)amino)-N-(4-methylpyridin-3-yl)pyrazine-2-carboxamide hydrochloride; 3-amino-6-chloro-5-(isobutyl(methyl)amino)-N-(5-methylisoxazol-3-yl)pyrazine-2-carboxamide hydrochloride; 3-amino-6-chloro-N-(6-chloro-5-methylpyridin-3-yl)-5-(isobutyl(methyl)amino)pyrazine-2-carboxamide bis(2,2,2-trifluoroacetate); 3-amino-N-benzyl-6-chloro-5-(isobutyl(methyl)amino)pyrazine-2-carboxamide 2,2,2-trifluoroacetate; 3-amino-6-chloro-N-(6-chloro-4-methylpyridin-3-yl)-5-(isobutyl(methyl)amino)pyrazine-2-carboxamide bis(2,2,2-trifluoroacetate); 3-amino-6-chloro-5-(isobutyl(methyl)amino)-N-(6-methoxy-4-methylpyridin-3-yl)pyrazine-2-carboxamide bis(2,2,2-trifluoroacetate); 3-amino-6-chloro-5-(isobutyl(methyl)amino)-N-phenylpyrazine-2-carboxamide; 3-amino-6-chloro-5-(isobutyl(methyl)amino)-N-(5-methylpyridin-3-yl)pyrazine-2-carboxamide bis(2,2,2-trifluoroacetate); 3-amino-6-chloro-N-(6-fluoro-4-methylpyridin-3-yl)-5-(isobutyl(methyl)amino)pyrazine-2-carboxamide bis(2,2,2-trifluoroacetate); 3-amino-6-chloro-5-(isobutyl(methyl)amino)-N-(piperidin-3-yl)pyrazine-2-carboxamide 2,2,2-trifluoroacetate; 3-amino-6-chloro-N-cyclohexyl-5-(isobutyl(methyl)amino)pyrazine-2-carboxamide 2,2,2-trifluoroacetate; 3-amino-6-chloro-5-(isobutyl(methyl)amino)-N-(piperidin-4-yl)pyrazine-2-carboxamide 2,2,2-trifluoroacetate; 3-amino-6-chloro-N-(cyclohexylmethyl)-5-(isobutyl(methyl)amino)pyrazine-2-carboxamide 2,2,2-trifluoroacetate; 3-amino-6-chloro-5-(isobutyl(methyl)amino)-N-(pyridin-3-ylmethyl)pyrazine-2-carboxamide 2,2,2-trifluoroacetate; 3-amino-6-chloro-5-(isobutyl(methyl)amino)-N-(pyridin-4-ylmethyl)pyrazine-2-carboxamide 2,2,2-trifluoroacetate; methyl 3-(3-amino-6-chloro-5-(isobutyl(methyl)amino)pyrazine-2-carboxamido)benzoate 2,2,2-trifluoroacetate; 5-(isobutyl(methyl)amino)-N-(4-methylpyridin-3-yl)pyrazine-2-carboxamide 2,2,2-trifluoroacetate; 3-amino-6-chloro-N-(4-methylpyridin-3-yl)-5-(piperidin-1-yl)pyrazine-2-carboxamide 2,2,2-trifluoroacetate; 3-amino-6-chloro-N-(4-methylpyridin-3-yl)-5-morpholinopyrazine-2-carboxamide 2,2,2-trifluoroacetate; 3-amino-6-chloro-N-(4-methylpyridin-3-yl)-5-(pyrrolidin-1-yl)pyrazine-2-carboxamide 2,2,2-trifluoroacetate; 3-amino-5-(benzyl(methyl)amino)-6-chloro-N-(4-methylpyridin-3-yl)pyrazine-2-carboxamide 2,2,2-trifluoroacetate; 3-amino-6-chloro-5-(diethylamino)-N-(4-methylpyridin-3-yl)pyrazine-2-carboxamide 2,2,2-trifluoroacetate; 3-amino-6-chloro-5-(isobutylamino)-N-(4-methylpyridin-3-yl)pyrazine-2-carboxamide 2,2,2-trifluoroacetate; 3-amino-6-chloro-5-(methyl(phenyl)amino)-N-(4-methylpyridin-3-yl)pyrazine-2-carboxamide 2,2,2-trifluoroacetate; 3-amino-6-chloro-5-(ethyl(methyl)amino)-N-(4-methylpyridin-3-yl)pyrazine-2-carboxamide 2,2,2-trifluoroacetate; and 6-chloro-5-(isobutyl(methyl)amino)-N-(4-methylpyridin-3-yl)pyrazine-2-carboxamide 2,2,2-trifluoroacetate.

Embodiments of the present invention can include prodrugs of 3-amino-N-carbamimidoyl-6-chloro-5-(isobutyl(methyl)amino)pyrazine-2-carboxamide, 3-amino-N-carbamimidoyl-6-chloro-5-(isobutyl(methyl)amino)pyrazine-2-carboxamide analogs, and compounds having a 3-amino-N-carbamimidoyl-6-chloro-5-(isobutyl(methyl)amino)pyrazine-2-carboxamide scaffold, and their isosteres, that are activated by liver enzymes (e.g., cyclic-1,3-propanyl esters substituted with groups that promote an oxidative cleavage reaction by CYP3A, etc.). 3-Amino-N-carbamimidoyl-6-chloro-5-(isobutyl(methyl)amino)pyrazine-2-carboxamide may also be referred to as 3,5-diamino-6-chloro-N-(diaminomethylidene)pyrazine-2-carboxamide. These modifications can render 3-amino-N-carbamimidoyl-6-chloro-5-(isobutyl(methyl)amino)pyrazine-2-carboxamide inactive or less active until activated in the liver (see, Current Opinion in Investigational Drugs 2006 Vol 7 No 2, 109-117; J. Med. Chem. 2008, 51, 2328-2345; and Nucleosides, Nucleotides, and Nucleic Acids, 24 (5-7):375-381, (2005), each of which is incorporated herein by reference for the corresponding discussion.

| | | | | Cell Viability | | 1b assay^{c} | hERG | |
|---|---|---|---|---|---|---|---|---|
| | | 2a (%)^{a} | | (%)^{b} | | IC50 | IC50 | |
| ID | Structure | 5 μM | 10 μM | 5 μM | 10 μM | (μM) | (μM) | Name |
| 148 | 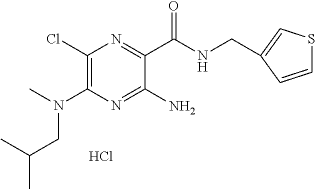 | 69 | 53 | 99 | 90 | | | 3-amino-6-chloro-5- (isobutyl(methyl)amino)- N-(thiophen-3-ylmethyl) pyrazine-2-carboxamide hydrochloride |
| 149 | 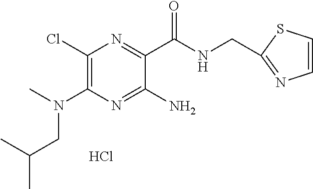 | 68 | 50 | >100 | >100 | | | 3-amino-6-chloro-5- (isobutyl(methyl)amino)- N-(thiazol-2-ylmethyl) pyrazine-2-carboxamide hydrochloride |
| 150 | 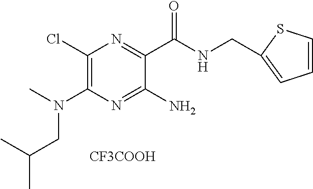 | 65 | 48 | >100 | 77 | | | 3-amino-6-chloro-5- (isobutyl(methyl)amino)- N-(thiophen-2-ylmethyl) pyrazine-2-carboxamide 2,2,2-trifluoroacetate |
| 200 | 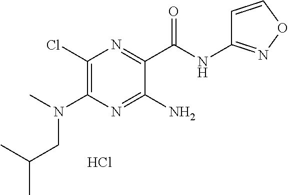 | 77 | 65 | 96 | 96 | | | 3-amino-6-chloro-5- (isobutyl(methyl)amino)- N-(isoxazol-3-yl)pyrazine- 2-carboxamide hydrochloride |
| 201 | 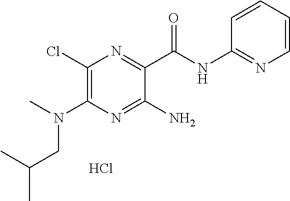 | 74 | 48 | >100 | 92 | | | 3-amino-6-chloro-5- (isobutyl(methyl)amino)- N-(pyridin-2-yl)pyrazine- 2-carboxamide hydrochloride |
| 202 | 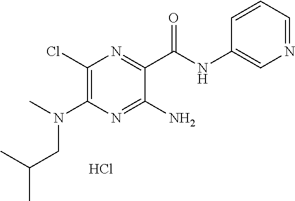 | 6 | 4 | 48 | 43 | | 1.5 | 3-amino-6-chloro-5- (isobutyl(methyl)amino)- N-(pyridin-3-yl)pyrazine- 2-carboxamide hydrochloride |
| 203 | 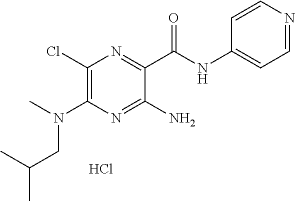 | 68 | 59 | 92 | 90 | | | 3-amino-6-chloro-5- (isobutyl(methyl)amino)- N-(pyridin-4-yl)pyrazine- 2-carboxamide hydrochloride |
| 204 | 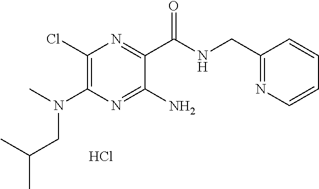 | 66 | 60 | >100 | 99 | | | 3-amino-6-chloro-5- (isobutyl(methyl)amino)- N-(pyridin-2-ylmethyl) pyrazine-2-carboxamide hydrochloride |
| 205 | 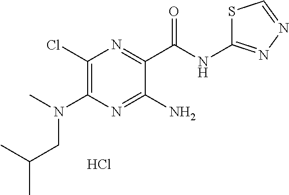 | 42 | 17 | 88 | 67 | | | 3-amino-6-chloro-5- (isobutyl(methyl)amino)- N-(1,3,4-thiadiazol-2-yl) pyrazine-2-carboxamide hydrochloride |
| 235 | 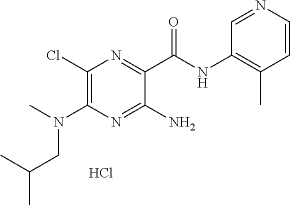 | 48 | 22 | 84 | 68 | | | 3-amino-6-chloro-5- (isobutyl(methyl)amino)- N-(4-methylpyridin-3-yl) pyrazine-2-carboxamide hydrochloride |
| 236 | 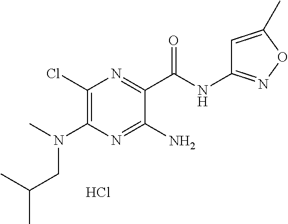 | 92 | 51 | 96 | 78 | | | 3-amino-6-chloro-5- (isobutyl(methyl)amino)- N-(5-methylisoxazol-3- yl)pyrazine-2-carboxamide hydrochloride |
| 291 | 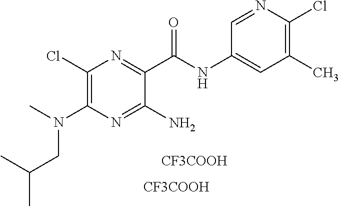 | 59 | 21 | 79 | 53 | | | 3-amino-6-chloro-N-(6- chloro-5-methylpyridin- 3-yl)-5-(isobutyl(methyl) amino)pyrazine-2- carboxamide bis(2,2,2-trifluoroacetate) |
| 292 | 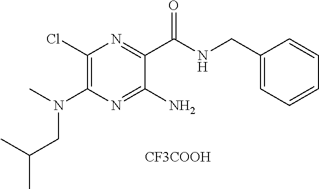 | 39 | 25 | 80 | 62 | inactive | | 3-amino-N-benzyl-6- chloro-5-(isobutyl(methyl) amino) pyrazine-2- carboxamide 2,2,2-trifluoroacetate |
| 293 | 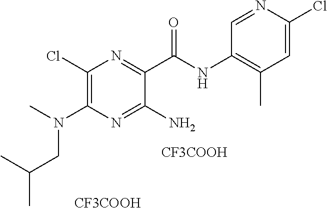 | 34 | 28 | 71 | 66 | inactive | | 3-amino-6-chloro-N- (6-chloro-4-methylpyridin- 3-yl)-5-(isobutyl(methyl) amino)pyrazine-2- carboxamide bis(2,2,2- trifluoroacetate) |
| 294 | 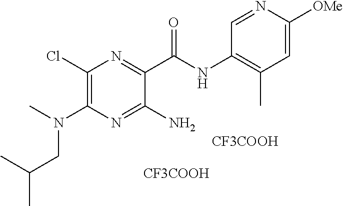 | 58 | 22 | 100 | 68 | | | 3-amino-6-chloro-5- (isobutyl(methyl)amino)-N- (6-methoxy-4- methylpyridin- 3-yl)pyrazine-2- carboxamide bis(2,2,2-trifluoroacetate) |
| 295 | 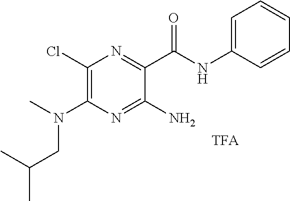 | 65 | 47 | >100 | 90 | | | 3-amino-6-chloro-5- (isobutyl(methyl)amino)- N-phenylpyrazine-2- carboxamide |
| 296 | 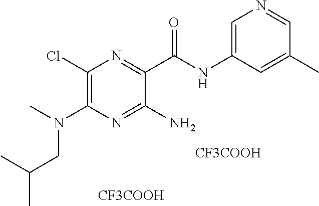 | 52 | 18 | 100 | 76 | | | 3-amino-6-chloro-5- (isobutyl(methyl)amino)- N-(5-methylpyridin-3-yl) pyrazine-2-carboxamide bis(2,2,2-trifluoroacetate) |
| 297 | 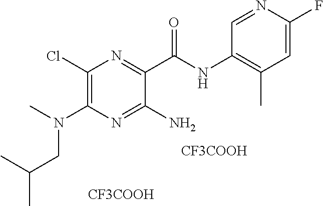 | 58 | 16 | 94 | 67 | | | 3-amino-6-chloro-N- (6-fluoro-4-methylpyridin- 3-yl)-5-(isobutyl(methyl) amino)pyrazine-2- carboxamide bis(2,2,2-trifluoroacetate) |
| 312 | 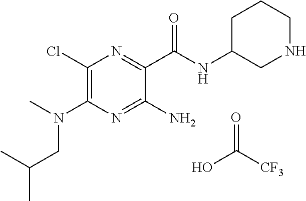 | 21 | 9 | 76 | 53 | 44 | 4 | 3-amino-6-chloro-5- (isobutyl(methyl)amino)-N- (piperidin-3-yl)pyrazine-2- carboxamide 2,2,2- trifluoroacetate |
| 313 | 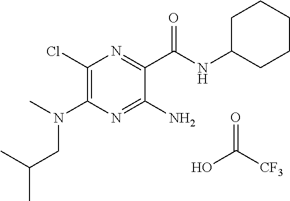 | 33 | 12 | 68 | 48 | >25 | | 3-amino-6-chloro-N- cyclohexyl-5- (isobutyl(methyl)amino) pyrazine-2-carboxamide 2,2,2-trifluoroacetate |
| 319 | 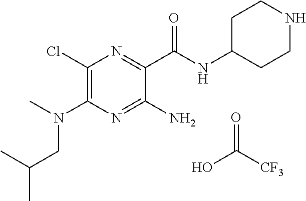 | 37 | 11 | 87 | 75 | 6.9 | | 3-amino-6-chloro-5- (isobutyl(methyl)amino)-N- (piperidin-4-yl)pyrazine-2- carboxamide 2,2,2- trifluoroacetate |
| 320 | 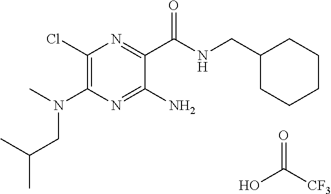 | 37 | 17 | 80 | 59 | inactive | | 3-amino-6-chloro-N- (cyclohexylmethyl)-5- (isobutyl(methyl)amino) pyrazine-2-carboxamide 2,2,2-trifluoroacetate |
| 341 | 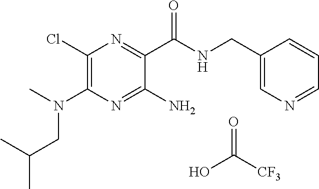 | 30 | 15 | 77 | 65 | inactive | | 3-amino-6-chloro-5- (isobutyl(methyl)amino)- N-(pyridin-3-ylmethyl) pyrazine-2-carboxamide 2,2,2-trifluoroacetate |
| 342 | 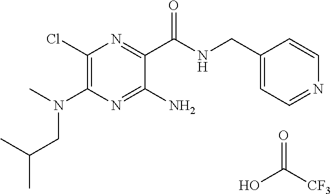 | 22 | 12 | 69 | 56 | inactive | | 3-amino-6-chloro-5- (isobutyl(methyl)amino)- N-(pyridin-4-ylmethyl) pyrazine-2-carboxamide 2,2,2- trifluoroacetate |
| 343 | 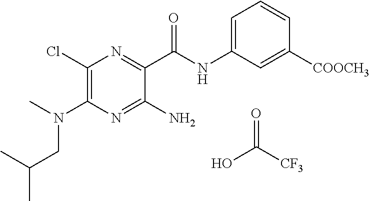 | 65 | 38 | 98 | 93 | | | methyl 3-(3-amino-6- chloro-5-(isobutyl(methyl) amino)pyrazine-2- carboxamido) benzoate 2,2,2- trifluoroacetate |
| 414 | 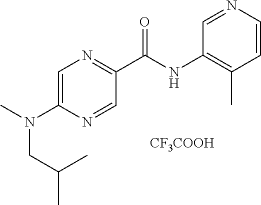 | 60 | 53 | 100 | 90 | | | 5-(isobutyl(methyl) amino)-N-(4-methyl- pyridin-3-yl)pyrazine- 2-carboxamide 2,2,2-trifluoroacetate |
| 415 | 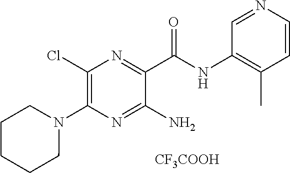 | 27 | 10 | 71 | 65 | inactive | | 3-amino-6-chloro-N-(4- methylpyridin-3-yl)- 5-(piperidin-1-yl)pyrazine- 2-carboxamide 2,2,2- trifluoroacetate |
| 419 | 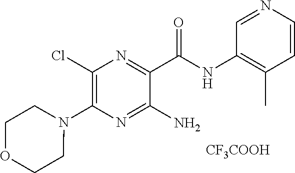 | 75 | 51 | 95 | 77 | | | 3-amino-6-chloro-N-(4- methylpyridin-3-yl)- 5-morpholinopyrazine-2- carboxamide 2,2,2-trifluoroacetate |
| 420 | 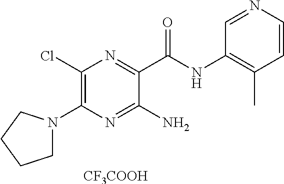 | 52 | 49 | 73 | 72 | | | 3-amino-6-chloro-N-(4- methylpyridin-3-yl)- 5-(pyrrolidin-1-yl) pyrazine-2-carboxamide 2,2,2-trifluoroacetate |
| 424 | 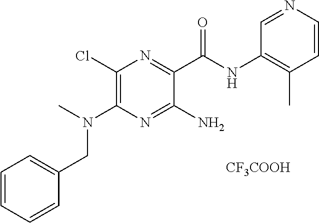 | 70 | 39 | 98 | 78 | | | 3-amino-5-(benzyl(methyl) amino)-6-chloro-N-(4- methylpyridin-3-yl) pyrazine-2-carboxamide 2,2,2-trifluoroacetate |
| 459 | 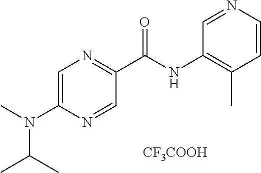 | 22 | 11 | 64 | 65 | | | 5-(isopropyl(methyl) amino)-N-(4-methyl- pyridin-3-yl) pyrazine-2-carboxamide 2,2,2-trifluoroacetate |
| 460 | 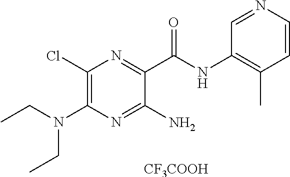 | 45 | 22 | 85 | 73 | | | 3-amino-6-chloro-5- (diethylamino)-N-(4- methylpyridin-3-yl) pyrazine-2-carboxamide 2,2,2-trifluoroacetate |
| 464 | 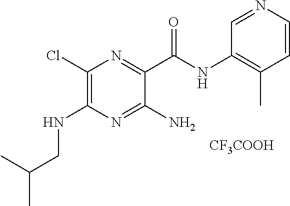 | 64 | 44 | 95 | 85 | | >10 | 3-amino-6-chloro-5- (isobutylamino)-N-(4- methylpyridin-3-yl) pyrazine-2-carboxamide 2,2,2-trifluoroacetate |
| 465 | 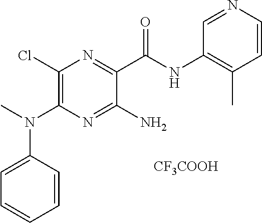 | 60 | 31 | 89 | 78 | | >10 | 3-amino-6-chloro-5- (methyl(phenyl)amino)-N- (4-methylpyridin-3-yl) pyrazine-2-carboxamide 2,2,2-trifluoroacetate |
| 469 | 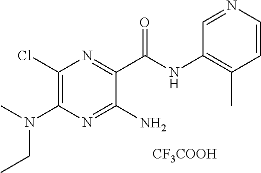 | 65 | 42 | 75 | 71 | | | 3-amino-6-chloro-5- (ethyl(methyl)amino)- N-(4-methylpyridin-3-yl) pyrazine-2-carboxamide 2,2,2-trifluoroacetate |
| 473 | 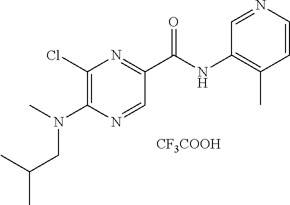 | 87 | 73 | 94 | 95 | | | 6-chloro-5- (isobutyl(methyl) amino)-N-(4- methylpyridin- 3-yl)pyrazine-2- carboxamide 2,2,2-trifluoroacetate |

^{a}Percent of virus activity remaining in the presence of the compound at the indicated concentration. The 2aHCV RNA replicon assay is performed as set forth in Example 4.
^{b}Viability of cells in the presence of the test compound, as assessed by alamar blue.
^{c}The half maximal inhibitory concentration (IC50) is a measure of the effectiveness of a compound in inhibiting viral replication. The 1b HCV RNA replicon assay uses the Huh7 cell line which contains an HCV 1b RNA replicon with a stable luciferase (LUC) reporter. This construct contains modifications that make the cell line more robust and provide stable LUC expression for antiviral screening. The LUC reporter is used as an indirect measure of HCV replication. The activity of the LUC reporter is directly proportional to HCV RNA levels and positive control antiviral compounds behave comparably using LUC endpoints.

### Pharmaceutical Compositions

The above-discussed compositions can be formulated using well-known reagents and methods. Compositions are provided in formulation with a pharmaceutically acceptable excipient(s). Wide varieties of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, an inhibitor is formulated in an aqueous buffer. Suitable aqueous buffers include, but are not limited to, acetate, succinate, citrate, and phosphate buffers varying in strengths from 5 mM to 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

In some embodiments, the inhibitor is formulated as a prodrug. The term “prodrug” refers to an inactive precursor of an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Harper, N.J. (1962). Drug Latentiation in Jucker, ed. Progress in Drug Research, 4:221-294; Morozowich et al. (1977). Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977). Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design. 5(4):265-287; Pauletti et al. (1997). Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of β-Lactam antibiotics, Pharm. Biotech. 11:345-365; Gaignault et al. (1996). Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, p. 185-218; Balant et al. (1990) Prodrugs for the improvement of drug absorption via different routes of administration, Eur. J. Drug Metab. Pharmacokinet., 15(2): 143-53; Balimane and Sinko (1999). Involvement of multiple transporters in the oral absorption of nucleoside analogues, Adv. Drug Delivery Rev., 39(1-3):183-209; Browne (1997). Fosphenyloin (Cerebyx), Clin. Neuropharmacol. 20(1): 1-12; Bundgaard (1979). Bioreversible derivatization of drugs—principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996). Improved oral drug delivery: solubility limitations overcome by the use of prodrugs, Adv. Drug Delivery Rev. 19(2): 115-130; Fleisher et al. (1985). Design of prodrugs for improved gastrointestinal absorption by intestinal enzyme targeting, Methods Enzymol. 112: 360-81; Farquhar D, et al. (1983). Biologically Reversible Phosphate-Protective Groups, J. Pharm. Sci., 72(3): 324-325; Han, H. K. et al. (2000). Targeted prodrug design to optimize drug delivery, AAPS PharmSci., 2(1): E6; Sadzuka Y. (2000). Effective prodrug liposome and conversion to active metabolite, Curr. Drug Metab., 1(1):31-48; D. M. Lambert (2000) Rationale and applications of lipids as prodrug carriers, Eur. J. Pharm. Sci., 11 Suppl 2:S15-27; Wang, W. et al. (1999) Prodrug approaches to the improved delivery of peptide drugs. Curr. Pharm. Des., 5(4):265-87.

Combination Therapies

Embodiments of the present invention include methods, inhibiting agents, and pharmaceutical formulations for the treatment of viral infection. Embodiments of the inhibiting agents and pharmaceutical formulations useful in the methods of the present disclosure can be employed in combination with other anti-viral agents to treat viral infection. In an embodiment, in accordance with the methods of the present invention, an inhibiting agent that is used to treat a host infected by a Flaviviridae family viral infection is used in combination with one or more other anti-HCV agents to treat HCV infection. In an embodiment, in accordance with the methods of the present invention, an inhibiting agent that inhibits the function of an HCV AH (also referred to herein as an “HCV AH function antagonist”) can be used in combination with one or more other anti-HCV agents to treat HCV infection.

Current medical practice to treat HCV infection typically employs either interferon-alpha monotherapy or combination therapy with ribavirin (such as Rebetol or Copegus) and either an interferon-alpha (such as interferon alpha 2b) or pegylated interferon (such as Pegasys, marketed by Roche, or PEG-Intron, marketed by Schering Plough). In accordance with the methods of the present disclosure, an inhibiting compound can be used in combination with these standard therapies to treat HCV infection.

A number of HCV protease inhibitors are in development for the treatment of HCV infection, and in accordance with the methods of the present disclosure, co-administration of an HCV AH function antagonist and an HCV protease inhibitor can be efficacious in the treatment of HCV. In one embodiment, an interferon alpha and/or a nucleoside analog such as ribavirin is/are also employed in this combination therapy. Suitable HCV protease inhibitors include, but are not limited to, telaprevir (VX-950, Vertex), BILN 2061 and BI12202 (Boehringer Ingelheim), boceprevir (SCH 503034, Schering Plough), ITMN191 (Roche/InterMune/Array BioPharma), MK-7009 (Merck), TMC435350 (Tibotec/Medivir), ACH-1095 and ACH-806 (Achillion/Gilead), and other inhibitors of NS3/NS4A protease, including, but not limited to, compounds in development by Presidio.

A number of HCV RNA polymerase (NS5B) inhibitors are in development for the treatment of HCV infection, and in accordance with the methods of the present disclosure, co-administration of an inhibiting agent that inhibits an HCV AH function and an HCV RNA polymerase inhibitor can be efficacious in the treatment of HCV. In one embodiment, an interferon alpha and/or a nucleoside analog such as ribavirin and/or an HCV protease inhibitor is/are also employed in this combination therapy. Suitable HCV RNA polymerase inhibitors include, but are not limited to, valopicitabine (NM283, Idenix/Novartis), HCV-796 (Wyeth/ViroPharma), R1626 (Roche), R7128 (Roche/Pharmasset), GS-9190 (Gilead), MK-0608 (Merck), PSI-6130 (Pharmasset), and PFE-868,554 (PFE).

A number of toll-like receptor (TLR) agonists are in development for the treatment of HCV infection, and in accordance with the methods of the present disclosure, co-administration of an HCV AH function antagonist and a TLR agonist can be efficacious in the treatment of HCV. In one embodiment, an interferon alpha and/or a nucleoside analog such as ribavirin and/or an HCV protease inhibitor and/or an HCV RNA polymerase inhibitor is/are also employed in this combination therapy. Suitable TLR agonists include, but are not limited to, TLR7 agonists (i.e., ANA245 and ANA975 (Anadys/Novartis)) and TLR9 agonists (i.e., Actilon (Coley) and IMO-2125 (Idera)).

A number of thiazolide derivatives are in development for the treatment of HCV infection, and in accordance with the methods of the present disclosure, co-administration of an HCV AH function antagonist and a thiazolide, including, but not limited to, Nitazoxanide (Alinia, or other sustained release formulations of nitazoxanide or other thiazolides, Romark Laboratories) can be efficacious in the treatment of HCV. In an embodiment, an interferon alpha and/or a nucleoside analog such as ribavirin and/or an HCV protease inhibitor and/or an HCV RNA polymerase inhibitor and/or a TLR agonist is/are also employed in this combination therapy.

In another embodiment of the methods of the present disclosure, co-administration of an HCV AH function antagonist and a cyclophilin inhibitor (i.e., NIM-811 (Novartis) and DEBIO-025 (Debiopharm)) and/or an alpha-glucosidase inhibitor (i.e., Celgosivir (Migenix)) and/or one or more agents from one or more of the other classes of HCV therapeutic agents discussed herein is used to treat HCV infection. Moreover, there are several targets within NS4B, and compounds that interact with these other targets can, in accordance with the methods of the present disclosure, be used in combination with an HCV AH function antagonist and, optionally, one or more of the other classes of inhibiting agents mentioned herein, to treat HCV infection. Such additional NS4B targets include: the N-terminal amphipathic helix (see PCT publication WO 2002/089731, incorporated herein by reference), the NS4B GTPase (see PCT publication WO 2005/032329, incorporated herein by reference), the binding activity to the 3′-UTR of HCV RNA (see PCT/US08/76806 and PCT/US08/76804 applications incorporated herein by reference), and the PIP2 binding activity of the first amphipathic helix in NS4B (see U.S. provisional patent application Ser. No. 60/057,188, incorporated herein by reference).

Other agents that can be used in combination with inhibiting agents of the present disclosure that inhibit AH function include (i) agents targeting NS5A, including, but not limited to, A-831 (Arrow Therapeutics), AZD2836 (Astra Zeneca), and agents in development by XTUPresidio or BMS (see PCT publications WO 2006/133326 and WO 2008/021928, incorporated herein by reference); (ii) agents targeting TBC1D20 and/or NS5A's interaction with TBC1D20 (see PCT publication WO 2007/018692 and U.S. patent application Ser. No. 11/844,993, incorporated herein by reference), (iii) agents targeting NS4B's GTPase activity (see PCT publication WO 2005/032329 and US patent application publication 2006/0199174, incorporated herein by reference); (iv) agents inhibiting membrane association mediated by the HCV amphipathic helices, such as those found in NS5A, NS4B, and NS5B (see PCT publication WO 2002/089731, supra), (v) agents targeting PIP2 or BAAPP domains in HCV proteins, such as those found in NS4B and NS5A (see U.S. provisional patent application 60/057,188, supra); (vi) agents targeting HCV entry, assembly, or release, including antibodies to co-receptors; (vii) agents targeting HCV NS3 helicase; (viii) siRNAs, shRNAs, antisense RNAs, or other RNA-based molecules targeting sequences in HCV; (ix) agents targeting microRNA122 or other microRNAs modulating HCV replication; (x) agents targeting PD-1, PD-L1, or PD-L2 interactions or pathway (see US patent application publications 20080118511, 20070065427, 20070122378, incorporated herein by reference); and (xi) agents targeting binding of NS4B to the 3′-UTR of HCV RNA, such as clemizole and its analogs (see PCT/US08/76806 and PCT/US08/76804 applications, incorporated herein by reference).

In another embodiment of the present disclosure, an inhibiting agent that prevents HCV AH function is used in combination with one or more drugs capable of treating an HIV infection to treat a patient that is co-infected with HIV and HCV. In another embodiment of the present disclosure, an inhibiting agent that inhibits HCV AH function is used in combination with one or more drugs capable of treating an HBV infection to treat a patient that is co-infected with HBV and HCV. In an embodiment, an inhibiting agent that inhibits HCV AH function is used in combination with a PD-L1 inhibitor to treat a viral infection.

As mentioned above, embodiments of the present include the administration of an inhibiting agent identified herein (or by using an embodiment of the screen of the invention) in conjunction with at least one additional therapeutic agent to treat a viral infection. Suitable additional therapeutic agents include, but are not limited to, ribavirin; a nucleoside analog (e.g., levovirin, viramidine, etc.); an NS3 inhibitor; an NS5 inhibitor; an interferon; and a side effect management agent.

In an embodiment, the at least one additional suitable therapeutic agent includes ribavirin. Ribavirin, 1-β-D-ribofuranosyl-1H-1,2,4-triazole-3-carboxamide, available from ICN Pharmaceuticals, Inc., Costa Mesa, Calif., is described in the Merck Index, compound No. 8199, Eleventh Edition. Its manufacture and formulation is described in U.S. Pat. No. 4,211,771. The disclosure also contemplates use of derivatives of ribavirin (see, e.g., U.S. Pat. No. 6,277,830).

In an embodiment, the at least one additional suitable therapeutic agent includes levovirin. Levovirin is the L-enantiomer of ribavirin, and exhibits the property of enhancing a Th1 immune response over a Th2 immune response. Levovirin is manufactured by ICN Pharmaceuticals.

In an embodiment, the at least one additional suitable therapeutic agent includes viramidine. Viramidine is a 3-carboxamidine derivative of ribavirin, and acts as a prodrug of ribavirin. It is efficiently converted to ribavirin by adenosine deaminases.

Nucleoside analogs that are suitable for use in a combination therapy include, but are not limited to, ribavirin, levovirin, viramidine, isatoribine, an L-ribofuranosyl nucleoside as disclosed in U.S. Pat. No. 5,559,101 and encompassed by Formula I of U.S. Pat. No. 5,559,101 (e.g., 1-β-L-ribofuranosyluracil, 1-β-L-ribofuranosyl-5-fluorouracil, 1-β-L-ribofuranosylcytosine, 9-β-L-ribofuranosyladenine, 9-β-L-ribofuranosylhypoxanthine, 9-β-L-ribofuranosylguanine, 9-β-L-ribofuranosyl-6-thioguanine, 2-amino-α-L-ribofuran[1,2′:4,5]oxazoline, O²,O²-anhydro-1-α-L-ribofuranosyluracil, 1-α-L-ribofuranosyluracil, 1-(2,3,5-tri-O-benzoyl-α-ribofuranosyl)-4-thiouracil, 1-α-L-ribofuranosylcytosine, 1-α-L-ribofuranosyl-4-thiouracil, 1-α-L-ribofuranosyl-5-fluorouracil, 2-amino-β-L-arabinofurano[1′,2′:4,5]oxazoline, O²,O²-anhydro-β-L-arabinofuranosyluracil, 2′-deoxy-β-L-uridine, 3′5′-Di-O-benzoyl-2′deoxy-4-thio 13-L-uridine, 2′-deoxy-β-L-cytidine, 2′-deoxy-β-L-4-thiouridine, 2′-deoxy-β-L-thymidine, 2′-deoxy-β-L-5-fluorouridine, 2′,3′-dideoxy-β-L-uridine, 2′-deoxy-β-L-5-fluorouridine, and 2′-deoxy-β-L-inosine); a compound as disclosed in U.S. Pat. No. 6,423,695 and encompassed by Formula I of U.S. Pat. No. 6,423,695; a compound as disclosed in U.S. Patent Publication No. 2002/0058635, and encompassed by Formula 1 of U.S. Patent Publication No. 2002/0058635; a nucleoside analog as disclosed in WO 01/90121 A2 (Idenix); a nucleoside analog as disclosed in WO 02/069903 A2 (Biocryst Pharmaceuticals Inc.); a nucleoside analog as disclosed in WO 02/057287 A2 or WO 02/057425 A2 (both Merck/Isis); and the like.

In an embodiment, the at least one additional suitable therapeutic agent can include HCV NS3 inhibitors. Suitable HCV non-structural protein-3 (NS3) inhibitors include, but are not limited to, a tri-peptide as disclosed in U.S. Pat. Nos. 6,642,204, 6,534,523, 6,420,380, 6,410,531, 6,329,417, 6,329,379, and 6,323,180 (Boehringer-Ingelheim); a compound as disclosed in U.S. Pat. No. 6,143,715 (Boehringer-Ingelheim); a macrocyclic compound as disclosed in U.S. Pat. No. 6,608,027 (Boehringer-Ingelheim); an NS3 inhibitor as disclosed in U.S. Pat. Nos. 6,617,309, 6,608,067, and 6,265,380 (Vertex Pharmaceuticals); an azapeptide compound as disclosed in U.S. Pat. No. 6,624,290 (Schering); a compound as disclosed in U.S. Pat. No. 5,990,276 (Schering); a compound as disclosed in Pause et al. (2003) J. Biol. Chem. 278:20374-20380; NS3 inhibitor BILN 2061 (Boehringer-Ingelheim; Lamarre et al. (2002) Hepatology 36:301 A; and Lamarre et al. (Oct. 26, 2003) Nature doi:10.1038/nature02099); NS3 inhibitor VX-950 (Vertex Pharmaceuticals; Kwong et al. (Oct. 24-28, 2003) 54^{th} Ann. Meeting AASLD); NS3 inhibitor SCH₆ (Abib et al. (Oct. 24-28, 2003) Abstract 137. Program and Abstracts of the 54^{th} Annual Meeting of the American Association for the Study of Liver Diseases (AASLD). Oct. 24-28, 2003. Boston, Mass.); any of the NS3 protease inhibitors disclosed in WO 99/07733, WO 99/07734, WO 00/09558, WO 00/09543, WO 00/59929 or WO 02/060926 (e.g., compounds 2, 3, 5, 6, 8, 10, 11, 18, 19, 29, 30, 31, 32, 33, 37, 38, 55, 59, 71, 91, 103, 104, 105, 112, 113, 114, 115, 116, 120, 122, 123, 124, 125, 126 and 127 disclosed in the table of pages 224-226 in WO 02/060926); an NS3 protease inhibitor as disclosed in any one of U.S. Patent Publication Nos. 2003019067, 20030187018, and 20030186895; and the like.

In an embodiment, the NS3 inhibitor used in a combination therapy of the invention is a member of the class of specific NS3 inhibitors, e.g., NS3 inhibitors that inhibit NS3 serine protease activity and that do not show significant inhibitory activity against other serine proteases such as human leukocyte elastase, porcine pancreatic elastase, or bovine pancreatic chymotrypsin, or cysteine proteases such as human liver cathepsin B.

In an embodiment, the at least one additional suitable therapeutic agent includes NS5B inhibitors. Suitable HCV non-structural protein-5 (NS5; RNA-dependent RNA polymerase) inhibitors include, but are not limited to, a compound as disclosed in U.S. Pat. No. 6,479,508 (Boehringer-Ingelheim); a compound as disclosed in any of International Patent Application Nos. PCT/CA02/01127, PCT/CA02/01128, and PCT/CA02/01129, all filed on Jul. 18, 2002 by Boehringer Ingelheim; a compound as disclosed in U.S. Pat. No. 6,440,985 (ViroPharma); a compound as disclosed in WO 01/47883, e.g., JTK-003 (Japan Tobacco); a dinucleotide analog as disclosed in Zhong et al. (2003) Antimicrob. Agents Chemother. 47:2674-2681; a benzothiadiazine compound as disclosed in Dhanak et al. (2002) J. Biol Chem. 277(41):38322-7; an NS5B inhibitor as disclosed in WO 02/100846 A1 or WO 02/100851 A2 (both Shire); an NS5B inhibitor as disclosed in WO 01/85172 A1 or WO 02/098424 A1 (both Glaxo SmithKline); an NS5B inhibitor as disclosed in WO 00/06529 or WO 02/06246 A1 (both Merck); an NS5B inhibitor as disclosed in WO 03/000254 (Japan Tobacco); an NS5B inhibitor as disclosed in EP 1 256,628 A2 (Agouron); JTK-002 (Japan Tobacco); JTK-109 (Japan Tobacco); and the like.

In an embodiment, the NS5 inhibitor used in the combination therapies of the invention is a member of the class of specific NS5 inhibitors, e.g., NS5 inhibitors that inhibit NS5 RNA-dependent RNA polymerase and that lack significant inhibitory effects toward other RNA dependent RNA polymerases and toward DNA dependent RNA polymerases.

In an embodiment, the at least one additional therapeutic agent is an interferon, e.g., interferon-alpha (IFN-α). Any known IFN-α can be used in the treatment methods of the invention. The term “interferon-alpha” as used herein refers to a family of related polypeptides that inhibit viral replication and cellular proliferation and modulate immune response. The term “IFN-α” includes naturally occurring IFN-α; synthetic IFN-α; derivatized IFN-α (e.g., PEGylated IFN-α, glycosylated IFN-α, and the like); and analogs of naturally occurring or synthetic IFN-α; essentially any IFN-α that has antiviral properties, as described for naturally occurring IFN-α.

Suitable alpha interferons include, but are not limited to, naturally-occurring IFN-α (including, but not limited to, naturally occurring IFN-α2a, IFN-α2b); recombinant interferon alpha-2b such as Intron-A interferon available from Schering Corporation, Kenilworth, N.J.; recombinant interferon alpha-2a such as Roferon interferon available from Hoffmann-La Roche, Nutley, N.J.; recombinant interferon alpha-2C such as Berofor alpha 2 interferon available from Boehringer Ingelheim Pharmaceutical, Inc., Ridgefield, Conn.; interferon alpha-n1, a purified blend of natural alpha interferons such as Sumiferon available from Sumitomo, Japan or as Wellferon interferon alpha-n1 (INS) available from the Glaxo-Wellcome Ltd., London, Great Britain; and interferon alpha-n3a mixture of natural alpha interferons made by Interferon Sciences and available from the Purdue Frederick Co., Norwalk, Conn., under the Alferon tradename.

The term “IFN-α” also encompasses consensus IFN-α. Consensus IFN-α (also referred to as “CIFN” and “IFN-con” and “consensus interferon”) encompasses, but is not limited to, the amino acid sequences designated IFN-con₁, IFN-con₂ and IFN-con₃ which are disclosed in U.S. Pat. Nos. 4,695,623 and 4,897,471; and consensus interferon as defined by determination of a consensus sequence of naturally occurring interferon alphas (e.g., Infergen®, InterMune, Inc., Brisbane, Calif.). IFN-con, is the consensus interferon agent in the Infergen® alfacon-1 product. The Infergen® consensus interferon product is referred to herein by its brand name (Infergen®) or by its generic name (interferon alfacon-1). DNA sequences encoding IFN-con may be synthesized as described in the aforementioned patents or other standard methods. In an embodiment, the at least one additional therapeutic agent is CIFN.

In an embodiment, fusion polypeptides comprising an IFN-α and a heterologous polypeptide can also be used in the combination therapies of the invention. Suitable IFN-α fusion polypeptides include, but are not limited to, Albuferon-alpha™ (a fusion product of human albumin and IFN-α; Human Genome Sciences; see, e.g., Osborn et al. (2002) J. Pharmacol. Exp. Therap. 303:540-548). Also suitable for use in the present disclosure are gene-shuffled forms of IFN-α. See., e.g., Masci et al. (2003) Curr. Oncol. Rep. 5:108-113. Other suitable interferons include), Multiferon (Viragen), Medusa Interferon (Flame) Technology), Locteron (Octopus), and Omega Interferon (Intarcia/Boehringer Ingelheim).

The term “IFN-α” also encompasses derivatives of IFN-α that are derivatized (e.g., are chemically modified relative to the naturally occurring peptide) to alter certain properties such as serum half-life. As such, the term “IFN-α” includes glycosylated IFN-α; IFN-α derivatized with polyethylene glycol (“PEGylated IFN-α”); and the like. PEGylated IFN-α, and methods for making same, is discussed in, e.g., U.S. Pat. Nos. 5,382,657; 5,981,709; and 5,951,974. PEGylated IFN-α encompasses conjugates of PEG and any of the above-described IFN-α molecules, including, but not limited to, PEG conjugated to interferon alpha-2a (Roferon, Hoffman La-Roche, Nutley, N.J.), interferon alpha 2b (Intron, Schering-Plough, Madison, N.J.), interferon alpha-2c (Berofor Alpha, Boehringer Ingelheim, Ingelheim, Germany); and consensus interferon as defined by determination of a consensus sequence of naturally occurring interferon alphas (Infergen®, InterMune, Inc., Brisbane, Calif.).

In an embodiment, the IFN-α polypeptides can be modified with one or more polyethylene glycol moieties, i.e., PEGylated. The PEG molecule of a PEGylated IFN-α polypeptide is conjugated to one or more amino acid side chains of the IFN-α polypeptide. In an embodiment, the PEGylated IFN-α contains a PEG moiety on only one amino acid. In another embodiment, the PEGylated IFN-α contains a PEG moiety on two or more amino acids, e.g., the IFN-α contains a PEG moiety attached to two, three, four, five, six, seven, eight, nine, or ten different amino acid residues. IFN-α may be coupled directly to PEG (i.e., without a linking group) through an amino group, a sulfhydryl group, a hydroxyl group, or a carboxyl group.

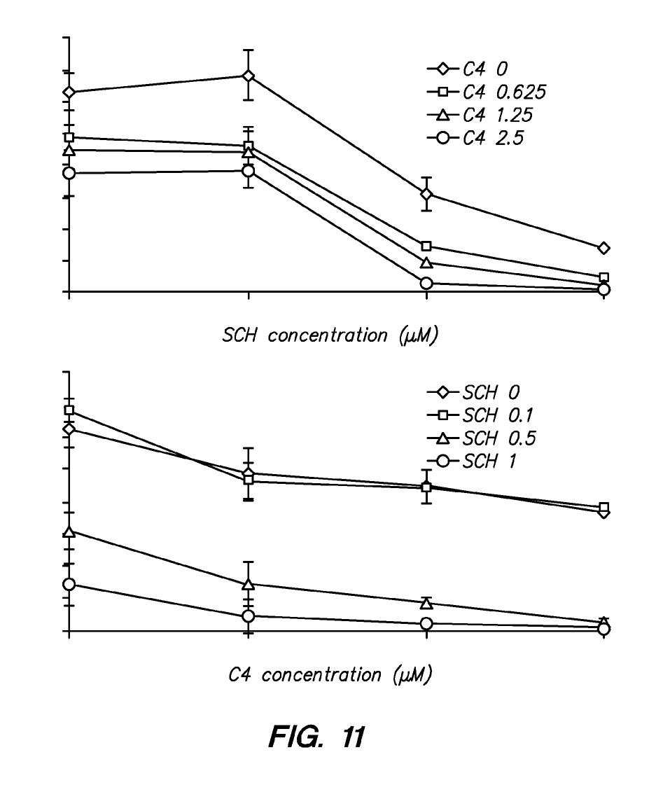

To determine the optimum combination of an HCV AH function inhibiting agent, such as 5-(N-Methyl-N-isobutyl)amiloride, with other anti-HCV agents, HCV replication assays and/or animal studies can be performed in the presence of various combinations of the various anti-HCV agents. Increased inhibition of replication in the presence of an additional agent (above that observed with monotherapy) is evidence for the potential benefit of the combination therapy. For example, HCV replication assays employing a luciferase reporter-linked HCV genome in the presence of various combinations of 3-amino-N-carbamimidoyl-6-chloro-5-(isobutyl(methyl)amino)pyrazine-2-carboxamidepyrazine-2-carboxamide and an NS3 protease inhibitor (SCH503034) are described elsewhere in this application and some results are shown in FIG. 11.

In some embodiments, the inhibitor and an antiviral agent, e.g. interferon, ribavirin, Enfuvirtide; RFI-641 (4,4″-bis-{4,6-bis-[3-(bis-carbamoylmethyl-sulfamoyl)-phenylamino]-(1,3,5)triazin-2-ylamino}-biphenyl-2,2″-disulfonic acid); BMS-433771 (2H-Imidazo[4,5-c]pyridin-2-one, 1-cyclopropyl-1,3-dihydro-3-((1-(3-hydroxypropyl)-1H-benzimidazol-2-yl)methyl)); arildone; Pleconaril (3-(3,5-Dimethyl-4-(3-(3-methyl-5-isoxazolyl)propoxy)phenyl)-5-(trifluoromethyl)-1,2,4-oxadiazole); Amantadine (tricyclo[3.3.1.1.3,7]decane-1-amine hydrochloride); Rimantadine (alpha-methyltricyclo[3.3.1.1.3,7]decane-1-methanamine hydrochloride); Acyclovir (acycloguanosine); Valaciclovir; Penciclovir (9-(4-hydroxy-3-hydroxymethyl-but-1-yl)guanine); Famciclovir (diacetyl ester of 9-(4-hydroxy-3-hydroxymethyl-but-1-yl)-6-deoxyguanine); Gancyclovir (9-(1,3-dihydroxy-2-propoxymethyl)guanine); Ara-A (adenosine arabinoside); Zidovudine (3′-azido-2′,3′-dideoxythymidine); Cidofovir (1-[(S)-3-hydroxy-2-(phosphonomethoxy)propyl]cytosine dihydrate); Dideoxyinosine (2′,3′-dideoxyinosine); Zalcitabine (2′,3′-dideoxycytidine); Stavudine (2′,3′-didehydro-2′,3′-dideoxythymidine); Lamivudine ((−)-β-L-3′-thia-2′,3′-dideoxycytidine); Abacavir (1S,4R)-4-[2-amino-6-(cyclopropylamino)-9H-purin-9-yl]-2-cyclopentene-1-methanol succinate); Emtricitabine (−)-β-L-3′-thia-2′,3′-dideoxy-5-fluorocytidine); Tenofovir disoproxil (Fumarate salt of bis(isopropoxycarbonyloxymethyl) ester of (R)-9-(2-phosphonylmethoxypropyl)adenine); Bromovinyl deoxyuridine (Brivudin); Iodo-deoxyuridine (Idoxuridine); Trifluorothymidine (Trifluridine); Nevirapine (11-cyclopropyl-5,11-dihydro-4-methyl-6H-dipyrido[3,2-b:2′,3′-f][1,4]diazepin-6-one); Delavirdine (1-(5-methanesulfonamido-1H-indol-2-yl-carbonyl)-4-[3-(1-methylethyl-amino)pyridinyl)piperazine monomethane sulfonated); Efavirenz ((−)-6-chloro-4-cyclopropylethynyl-4-trifluoromethyl-1,4-dihydro-2H-3,1-benzoxazin-2-one); Foscarnet (trisodium phosphonoformate); Ribavirin (1-β-D-ribofuranosyl-1H-1,2,4-triazole-3-carboxamide); Raltegravir (N-[(4-Fluorophenyl)methyl]-1,6-dihydro-5-hydroxy-1-methyl-2-[1-methyl-1-[[(5-methyl-1,3,4-oxadiazol-2-yl)carbonyl]amino]ethyl]-6-oxo-4-pyrimidinecarboxamide monopotassium salt); Neplanocin A; Fomivirsen; Saquinavir (SQ); Ritonavir ([5S-(5R,8R,10R,11R)]-10-hydroxy-2-methyl-5-(1-methylethyl)-1-[2-(methylethyl)-4-thiazolyl]-3,6-dioxo-8,11-bis(phenylmethyl)-2,4,7,12-tetraazamidecan-13-oic acid 5-thiazolylmethyl ester); Indinavir ([(1S,2R,5(S)-2,3,5-trideoxy-N-(2,3-dihydro-2-hydroxy-1H-inden-1-yl)-5-[2-[[(1,1-dimethylethyl)amino]carbonyl]-4-pyridinylmethyl)-1-piperazinyl]-2-(phenylmethyl-erythro)pentonamide); Amprenavir; Nelfinavir; Lopinavir; Atazanavir; Bevirimat; Indinavir; Relenza; Zanamivir; Oseltamivir; Tarvacin; agents targeting PIP2 or BAAPP domains in HCV proteins, such as those found in NS4B and NS5A (see U.S. provisional patent application 60/057,188), clemizole or its derivatives or analogs, nitazxoxanide, a thiazolide, or sustained release formulations of the above, etc. are administered to individuals in a formulation (e.g., in the same or in separate formulations) with a pharmaceutically acceptable excipient(s). The therapeutic HCV AH function antagonist and second antiviral agent, as well as additional therapeutic agents as described herein for combination therapies, can be administered orally, subcutaneously, intramuscularly, parenterally, or other route. HCV AH function antagonist and second antiviral agent may be administered by the same route of administration or by different routes of administration. The therapeutic agents can be administered by any suitable means including, but not limited to, for example, oral, rectal, nasal, topical (including transdermal, aerosol, buccal and sublingual), vaginal, parenteral (including subcutaneous, intramuscular, intravenous and intradermal), intravesical or injection into an affected organ.

The therapeutic agent(s) may be administered in a unit dosage form and may be prepared by any methods well known in the art. Such methods include combining the compounds of the present invention with a pharmaceutically acceptable carrier or diluent which constitutes one or more accessory ingredients. A pharmaceutically acceptable carrier is selected on the basis of the chosen route of administration and standard pharmaceutical practice. Each carrier must be “pharmaceutically acceptable” in the sense of being compatible with the other ingredients of the formulation and not injurious to the subject. This carrier can be a solid or liquid and the type is generally chosen based on the type of administration being used.

Examples of suitable solid carriers include lactose, sucrose, gelatin, agar and bulk powders. Examples of suitable liquid carriers include water, pharmaceutically acceptable fats and oils, alcohols or other organic solvents, including esters, emulsions, syrups or elixirs, suspensions, solutions and/or suspensions, and solution and or suspensions reconstituted from non-effervescent granules and effervescent preparations reconstituted from effervescent granules. Such liquid carriers may contain, for example, suitable solvents, preservatives, emulsifying agents, suspending agents, diluents, sweeteners, thickeners, and melting agents. Preferred carriers are edible oils, for example, corn or canola oils. Polyethylene glycols, e.g. PEG, are also good carriers.

Any drug delivery device or system that provides for the dosing regimen of the instant invention can be used. A wide variety of delivery devices and systems are known to those skilled in the art.

Although such may not be necessary, agents described herein can optionally be targeted to the liver, using any known targeting means. The inhibitors of the invention may be formulated with a wide variety of compounds that have been demonstrated to target compounds to hepatocytes. Such liver targeting compounds include, but are not limited to, asialoglycopeptides; basic polyamino acids conjugated with galactose or lactose residues; galactosylated albumin; asialoglycoprotein-poly-L-lysine) conjugates; lactosaminated albumin; lactosylated albumin-poly-L-lysine conjugates; galactosylated poly-L-lysine; galactose-PEG-poly-L-lysine conjugates; lactose-PEG-poly-L-lysine conjugates; asialofetuin; and lactosylated albumin.

The terms “targeting to the liver” and “hepatocyte targeted” refer to targeting of an agent to a hepatocyte, particularly a virally infected hepatocyte, such that at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, or more, of the protease inhibitor agent administered to the subject enters the liver via the hepatic portal and becomes associated with (e.g., is taken up by) a hepatocyte. As mentioned, above, targeting to the liver can be achieved by modifying the inhibitory agents to create prodrugs that are activated by liver enzymes (e.g., cyclic-1,3-propanyl esters substituted with groups that promote an oxidative cleavage reaction by CYP3A, etc.). These modifications can render the agents inactive or less active until activated in the liver (see, Current Opinion in Investigational Drugs 2006 Vol 7 No 2, 109-117; J. Med. Chem. 2008, 51, 2328-2345; and Nucleosides, Nucleotides, and Nucleic Acids, 24 (5-7):375-381, (2005), each of which is incorporated herein by reference for the corresponding discussion.

HCV infection is associated with liver fibrosis and in certain embodiments the inhibitors may by useful in treating liver fibrosis (particularly preventing, slowing of progression, etc.). The methods involve administering an inhibitor of the invention as described above, in an amount effective to reduce viral load, thereby treating liver fibrosis in the subject. Treating liver fibrosis includes reducing the risk that liver fibrosis will occur; reducing a symptom associated with liver fibrosis; and increasing liver function.

Whether treatment with an agent as described herein is effective in reducing liver fibrosis is determined by any of a number of well-established techniques for measuring liver fibrosis and liver function. The benefit of anti-fibrotic therapy can be measured and assessed by using the Child-Pugh scoring system which comprises a multi-component point system based upon abnormalities in serum bilirubin level, serum albumin level, prothrombin time, the presence and severity of ascites, and the presence and severity of encephalopathy. Based upon the presence and severity of abnormality of these parameters, patients may be placed in one of three categories of increasing severity of clinical disease: A, B, or C.

Treatment of liver fibrosis (e.g., reduction of liver fibrosis) can also be determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems.

The METAVIR scoring system is based on an analysis of various features of a liver biopsy, including fibrosis (portal fibrosis, centrilobular fibrosis, and cirrhosis); necrosis (piecemeal and lobular necrosis, acidophilic retraction, and ballooning degeneration); inflammation (portal tract inflammation, portal lymphoid aggregates, and distribution of portal inflammation); bile duct changes; and the Knodell index (scores of periportal necrosis, lobular necrosis, portal inflammation, fibrosis, and overall disease activity). The definitions of each stage in the METAVIR system are as follows: score: 0, no fibrosis; score: 1, stellate enlargement of portal tract but without septa formation; score: 2, enlargement of portal tract with rare septa formation; score: 3, numerous septa without cirrhosis; and score: 4, cirrhosis.

Knodell's scoring system, also called the Hepatitis Activity Index, classifies specimens based on scores in four categories of histologic features: I. Periportal and/or bridging necrosis; II. Intralobular degeneration and focal necrosis; III. Portal inflammation; and IV. Fibrosis. In the Knodell staging system, scores are as follows: score: 0, no fibrosis; score: 1, mild fibrosis (fibrous portal expansion); score: 2, moderate fibrosis; score: 3, severe fibrosis (bridging fibrosis); and score: 4, cirrhosis. The higher the score, the more severe the liver tissue damage. Knodell (1981) Hepatol. 1:431.

In the Scheuer scoring system scores are as follows: score: 0, no fibrosis; score: 1, enlarged, fibrotic portal tracts; score: 2, periportal or portal-portal septa, but intact architecture; score: 3, fibrosis with architectural distortion, but no obvious cirrhosis; score: 4, probable or definite cirrhosis. Scheuer (1991) J. Hepatol. 13:372.

The Ishak scoring system is described in Ishak (1995) J. Hepatol. 22:696-699. Stage 0, No fibrosis; Stage 1, Fibrous expansion of some portal areas, with or without short fibrous septa; stage 2, Fibrous expansion of most portal areas, with or without short fibrous septa; stage 3, Fibrous expansion of most portal areas with occasional portal to portal (P-P) bridging; stage 4, Fibrous expansion of portal areas with marked bridging (P-P) as well as portal-central (P-C); stage 5, Marked bridging (P-P and/or P-C) with occasional nodules (incomplete cirrhosis); stage 6, Cirrhosis, probable or definite.

In some embodiments, a therapeutically effective amount of an agent of the invention is an amount of agent that effects a change of one unit or more in the fibrosis stage based on pre- and post-therapy measures of liver function (e.g, as determined by biopsies). In particular embodiments, a therapeutically effective amount of an inhibitor reduces liver fibrosis by at least one unit in the Child-Pugh, METAVIR, the Knodell, the Scheuer, the Ludwig, or the Ishak scoring system.

Secondary, or indirect, indices of liver function can also be used to evaluate the efficacy of treatment. Morphometric computerized semi-automated assessment of the quantitative degree of liver fibrosis based upon specific staining of collagen and/or serum markers of liver fibrosis can also be measured as an indication of the efficacy of a subject treatment method. Secondary indices of liver function include, but are not limited to, serum transaminase levels, prothrombin time, bilirubin, platelet count, portal pressure, albumin level, and assessment of the Child-Pugh score. An effective amount of an agent is an amount that is effective to increase an index of liver function by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the index of liver function in an untreated individual, or to a placebo-treated individual. Those skilled in the art can readily measure such indices of liver function, using standard assay methods, many of which are commercially available, and are used routinely in clinical settings.

Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase.

A therapeutically effective amount of an agent is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Those skilled in the art can readily measure such serum markers of liver fibrosis, using standard assay methods, many of which are commercially available, and are used routinely in clinical settings. Methods of measuring serum markers include immunological-based methods, e.g., enzyme-linked immunosorbent assays (ELISA), radioimmunoassays, and the like, using antibody specific for a given serum marker.

Qualitative or quantitative tests of functional liver reserve can also be used to assess the efficacy of treatment with an agent. These include: indocyanine green clearance (ICG), galactose elimination capacity (GEC), aminopyrine breath test (ABT), antipyrine clearance, monoethylglycine-xylidide (MEG-X) clearance, and caffeine clearance.

As used herein, a “complication associated with cirrhosis of the liver” refers to a disorder that is a sequellae of decompensated liver disease, i.e., or occurs subsequently to and as a result of development of liver fibrosis, and includes, but it not limited to, development of ascites, variceal bleeding, portal hypertension, jaundice, progressive liver insufficiency, encephalopathy, hepatocellular carcinoma, liver failure requiring liver transplantation, and liver-related mortality.

A therapeutically effective amount of an agent in this context can be regarded as an amount that is effective in reducing the incidence (e.g., the likelihood that an individual will develop) of a disorder associated with cirrhosis of the liver by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to an untreated individual, or to a placebo-treated individual.

Whether treatment with an agent is effective in reducing the incidence of a disorder associated with cirrhosis of the liver can readily be determined by those skilled in the art.

Reduction in HCV viral load, as well as reduction in liver fibrosis, can be associated with an increase in liver function. Thus, the invention provides methods for increasing liver function, generally involving administering a therapeutically effective amount of an agent of the invention. Liver functions include, but are not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

Whether a liver function is increased is readily ascertainable by those skilled in the art, using well-established tests of liver function. Thus, synthesis of markers of liver function such as albumin, alkaline phosphatase, alanine transaminase, aspartate transaminase, bilirubin, and the like, can be assessed by measuring the level of these markers in the serum, using standard immunological and enzymatic assays. Splanchnic circulation and portal hemodynamics can be measured by portal wedge pressure and/or resistance using standard methods. Metabolic functions can be measured by measuring the level of ammonia in the serum.

Whether serum proteins normally secreted by the liver are in the normal range can be determined by measuring the levels of such proteins, using standard immunological and enzymatic assays. Those skilled in the art know the normal ranges for such serum proteins. The following are non-limiting examples. The normal range of alanine transaminase is from about 7 to about 56 units per liter of serum. The normal range of aspartate transaminase is from about 5 to about 40 units per liter of serum. Bilirubin is measured using standard assays. Normal bilirubin levels are usually less than about 1.2 mg/dL. Serum albumin levels are measured using standard assays. Normal levels of serum albumin are in the range of from about 35 to about 55 g/L. Prolongation of prothrombin time is measured using standard assays. Normal prothrombin time is less than about 4 seconds longer than control.

A therapeutically effective amount of an agent in this context is one that is effective to increase liver function by at least about 10%, at least about 20%, at least about 30%, at least about 40%, at least about 50%, at least about 60%, at least about 70%, at least about 80%, or more. For example, a therapeutically effective amount of an agent is an amount effective to reduce an elevated level of a serum marker of liver function by at least about 10%, at least about 20%, at least about 30%, at least about 40%, at least about 50%, at least about 60%, at least about 70%, at least about 80%, or more, or to reduce the level of the serum marker of liver function to within a normal range. A therapeutically effective amount of an agent is also an amount effective to increase a reduced level of a serum marker of liver function by at least about 10%, at least about 20%, at least about 30%, at least about 40%, at least about 50%, at least about 60%, at least about 70%, at least about 80%, or more, or to increase the level of the serum marker of liver function to within a normal range.

HCV infection is associated with hepatic cancer and in certain embodiments the present invention provides compositions and methods of reducing the risk that an individual will develop hepatic cancer. The methods involve administering an agent, as describe above, wherein viral load is reduced in the individual, and wherein the risk that the individual will develop hepatic cancer is reduced. An effective amount of an agent is one that reduces the risk of hepatic cancer by at least about 10%, at least about 20%, at least about 25%, at least about 30%, at least about 35%, at least about 40%, at least about 45%, at least about 50%, at least about 55%, at least about 60%, at least about 65%, at least about 70%, or more. Whether the risk of hepatic cancer is reduced can be determined in, e.g., study groups, where individuals treated according to the methods of the invention have reduced incidence of hepatic cancer.

### Subjects Amenable to Treatment Using the Agents of the Invention

Individuals who have been clinically diagnosed as infected with a virus, particularly HCV, are suitable for treatment with the methods of the present invention. Individuals who are infected with HCV are generally identified (diagnosed) as having HCV RNA in their blood, and/or having anti-HCV antibody in their serum. The patient may be infected with any HCV genotype (genotype 1, including 1a and 1b, 2, 3, 4, 6, etc. and subtypes (e.g., 2a, 2b, 3a, etc.)), particularly a difficult to treat genotype such as HCV genotype 1, or other HCV subtypes and quasispecies. Such individuals include naïve individuals (e.g., individuals not previously treated for HCV) and individuals who have failed prior treatment for HCV (“treatment failure” patients). Treatment failure patients include non-responders (e.g., individuals in whom the HCV titer was not significantly or sufficiently reduced by a previous antiviral treatment for HCV); and relapsers (e.g., individuals who were previously treated for HCV, whose HCV titer decreased, and subsequently increased). In particular embodiments of interest, individuals of interest for treatment according to the invention have detectable HCV titer indicating active viral replication, they may also have an HCV titer of at least about 10⁵, at least about 5×10⁵, or at least about 10⁶, or greater than 2 million genome copies of HCV per milliliter of serum.

### Determining Effectiveness of Antiviral Treatment

Whether a subject method is effective in treating a hepatitis virus infection, particularly an HCV infection, can be determined by measuring viral load, or by measuring a parameter associated with HCV infection, including, but not limited to, liver fibrosis.

Viral load can be measured by measuring the titer or level of virus in serum. These methods include, but are not limited to, a quantitative polymerase chain reaction (PCR) and a branched DNA (bDNA) test. For example, quantitative assays for measuring the viral load (titer) of HCV RNA have been developed. Many such assays are available commercially, including a quantitative reverse transcription PCR(RT-PCR) (Amplicor HCV Monitor™ Roche Molecular Systems, New Jersey); and a branched DNA (deoxyribonucleic acid) signal amplification assay (Quantiplex™ HCV RNA Assay (bDNA), Chiron Corp., Emeryville, Calif.). See, e.g., Gretch et al. (1995) Ann. Intern. Med. 123:321-329.

As noted above, whether a subject method is effective in treating a hepatitis virus infection, e.g., an HCV infection, can be determined by measuring a parameter associated with hepatitis virus infection, such as liver fibrosis. Liver fibrosis reduction can be assessed by a variety of serum-based assay or by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems. Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase.

As one non-limiting example, levels of serum alanine aminotransferase (ALT) are measured, using standard assays. In general, an ALT level of less than about 45 international units per milliliter serum is considered normal. In some embodiments, an effective amount of anti-HCV agent is an amount effective to reduce ALT levels to less than about 45 IU/ml serum.

### EXPERIMENTAL

### Example 1

### Assays for Detecting Inhibitors of HCV AH Function

DLS-based screens for inhibitors of HCV amphipathic helix (AH) function. The N-terminal amphipathic helices (AHs) in NS4B and NS5A mediate membrane association and have been genetically validated as essential for HCV genome replication (Elazar et al. J. Virol. 2003, Elazar et al. J. Virol. 2004). We have discovered that these AHs not only mediate membrane association, but have functional biochemical activities. In particular they induce changes in the physical properties of lipid vesicles that result in an increase in the apparent average diameter of the vesicles, as measured by dynamic light scattering. This, in turn, enables novel screening assays based on this discovery that can identify pharmacologic inhibitors of HCV AH function that can be used to inhibit HCV replication.

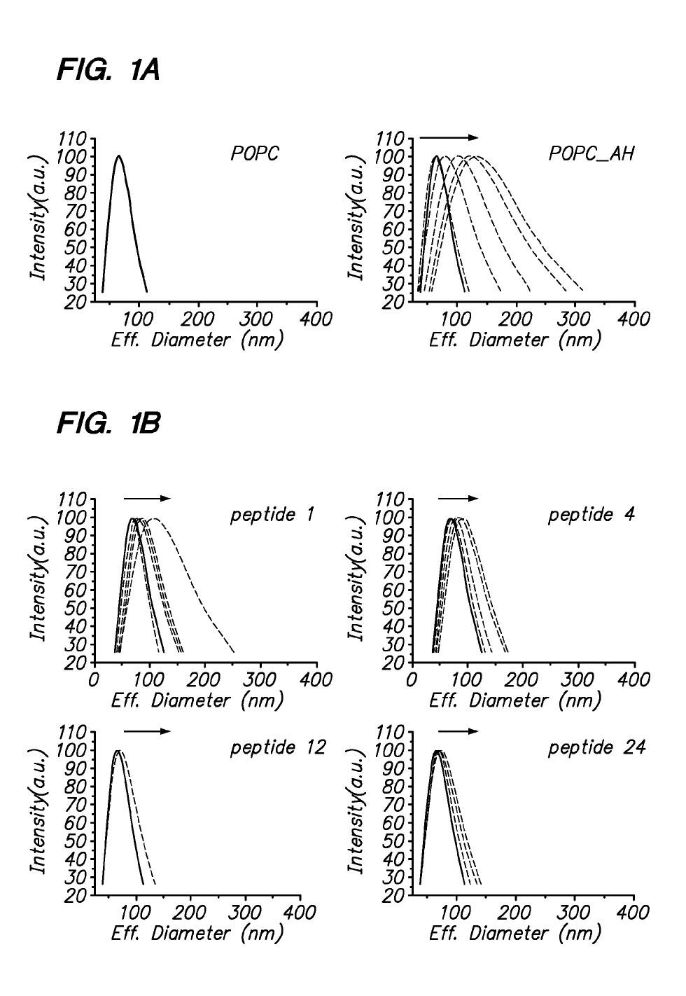

The NS5A AH induces changes in the apparent size of lipid vesicles, as measured by DLS. Small unilamellar lipid vesicles of 1-palmitoyl-2-oleoyl-sn-glycero-3-phosphocholine (Avanti Polar Lipids) were prepared by the extrusion method described in the art, for example, in Cho et al., Journal of Virology, 81(12): 6682-6689, 2007. Throughout the experiments, 10 mM Tris (pH 7.5) and 150 mM NaCl solution with 1 mM ethylene diamine tetraacetic acid (EDTA) in 18.2 MΩ-cm MilliQ water (MilliPore) was used. This method yielded a population of relatively uniformly sized vesicles. The size distribution of the latter was confirmed by dynamic light scattering (DLS). Methods for using dynamic light scattering are described in the art, for example, in Pencer et al. Biophys. J., 81(5):2716-2728, 2001; Pencer and Hallett, Langmuir, 19:7488-7497, 2003. A synthetic peptide corresponding to the NS5A amphipathic helix was then added to the lipid vesicles, while monitoring by DLS. A large increase in the average size of the vesicle population occurs as a function of time (FIG. 1a). This unanticipated finding is the first time this effect of an HCV AH on lipid vesicles has been observed. It also highlights the potential of a novel assay to identify inhibitors of AH function based on AH-induced changes in DLS (FIG. 1b).

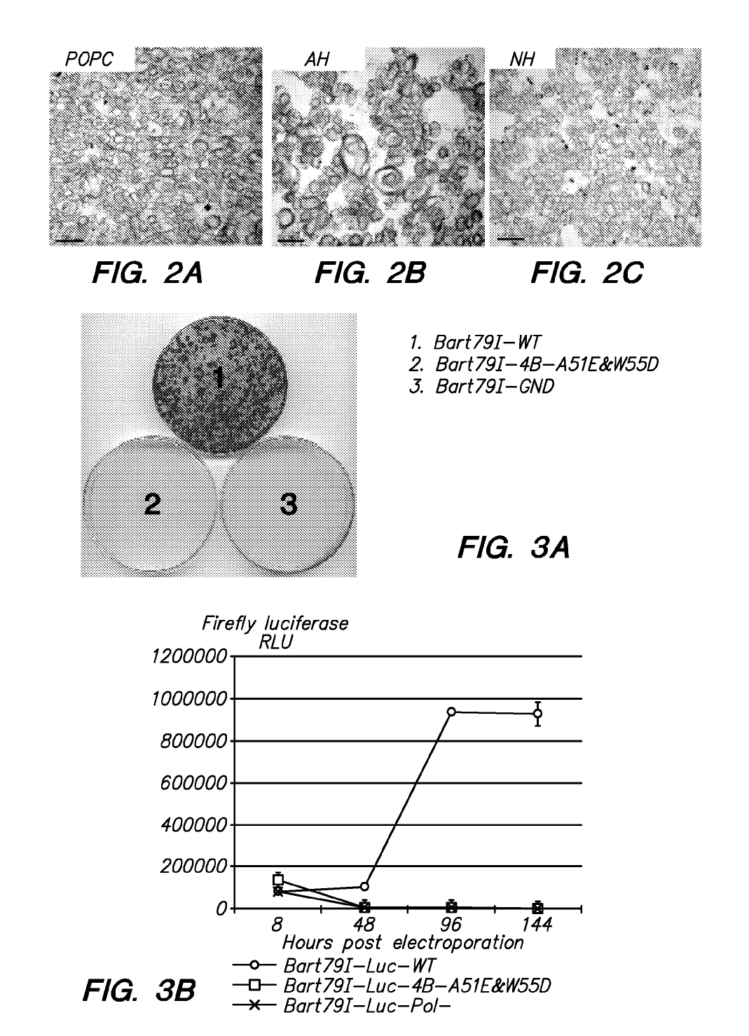

EM data suggests the mechanism of NS5A AH induced DLS changes involves lysis and fusion to form larger size vesicles. EM analysis of samples from the above experiments revealed an apparent increase in the diameter of the vesicles as well as the creation of apparent multilamellar vesicles, indicating that the increase in vesicle size on DLS is presumably due to AH-mediated vesicle lysis followed by fusion to create larger sized vesicles (FIG. 2).

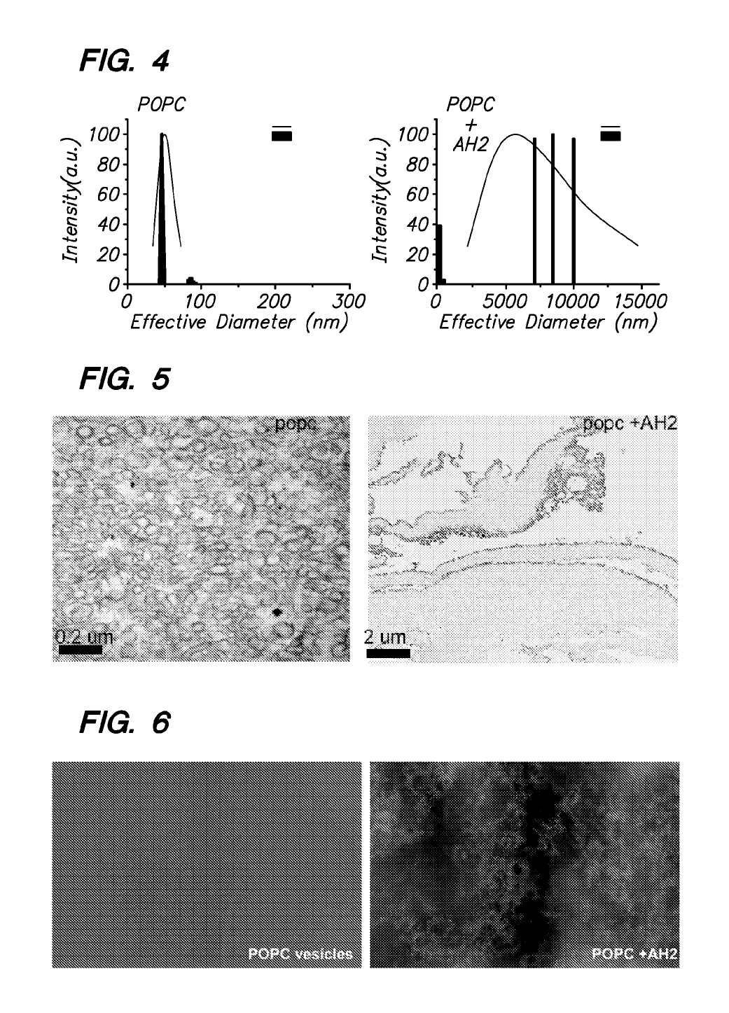

DLS can be used to assay the function of other HCV amphipathic helices. The HCV non-structural protein NS4B also harbors amphipathic helices. For example, in addition to a previously-described N-terminal AH, NS4B has a second downstream AH, which we term 4BAH2. As shown in FIG. 3, mutation of 4BAH2 abrogates HCV genome replication. This genetically validates the AH2 as a novel anti-HCV target. 4BAH2 function can also be assayed using the above DLS assay. As shown in FIG. 4, 4BAH2 induces a large increase in the apparent average size of lipid vesicles.

4BAH2 induces vesicle aggregation. The above increase in apparent vesicle size indicated by the DLS assay could be due to either fusion of vesicles or aggregation of vesicles. As shown in FIG. 5, electron microscopic analysis reveals that 4BAH2 induces predominantly aggregation of vesicles.

Identification of a novel class of HCV inhibitors targeting the NS4B AH2. The above aggregation of lipid vesicles suggested another type of assay for AH function. Lipid vesicles were prepared as above, but a fluorescent lipid was added during their formation. The ImageXpressMICRO™ imaging system was utilized to enable automated acquisition and analysis of images for high throughput synthetic lipid vesicle-based screening. ImageXpressMICRO is powered by MetaXpress™ cellular image analysis software for high content screening assays. Using MetaXpress, one can readily develop custom protocols to fit the analysis of aggregation, such as that indicated in the materials and methods section. In addition, the dramatic size of 4BAH2-induced aggregation can be readily visualized by simple inspection.

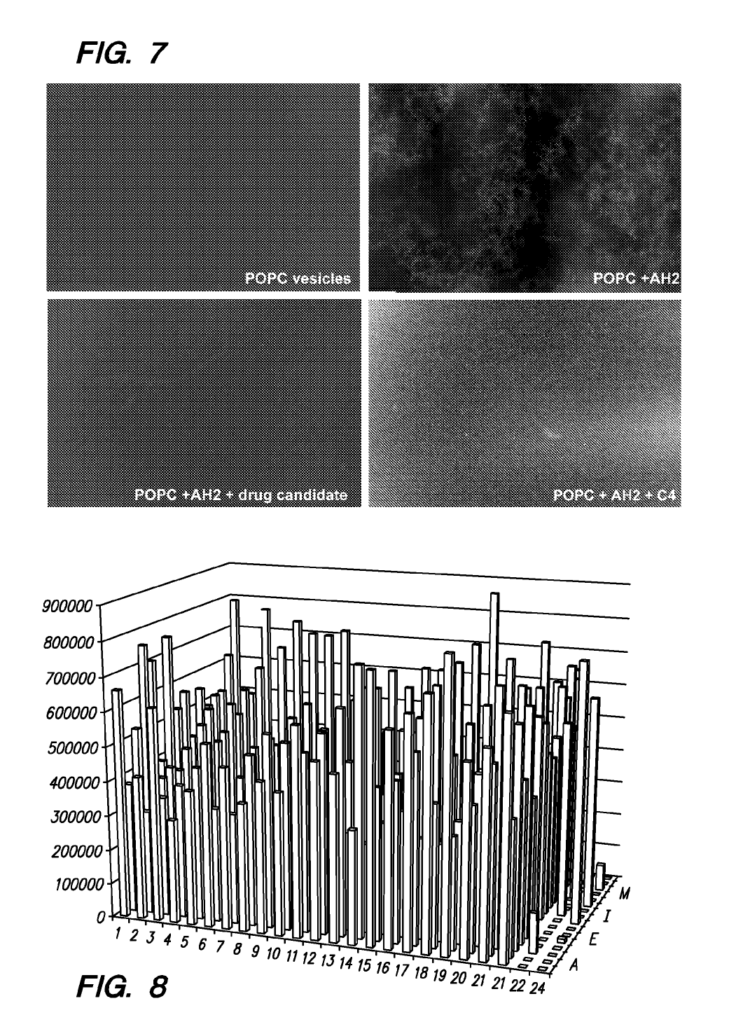

The inverted fully automated epifluorescent microscope is designed for scanning standard multi-well microplates or slides, for end-point assays. ImageXpressMICRO features image-based auto-focus and optional high-speed laser auto-focus for increased throughput. The high precision design provides better than ±100 nm resolution from its fully automated stage and focus control. As shown in FIG. 6, upon addition of 4BAH2, the resulting aggregations of lipid vesicles can be visualized with a fluorescent microscope.

### Example 2

### High-Throughput Screens for Inhibitors of HCV AH Function

Fluorescence based screen. Visualization of aggregation of lipid vesicles comprising fluorescent lipids was then adapted to a 384 well plate format, and used to screen a small molecule library for inhibitors of 4BAH2. Simple inspection for the presence of aggregates or their absence can identify positive and negative hits, respectively (see FIG. 7). In addition, the images can be digitized and quantitatively analyzed for the amount of fluorescence contained within a specified pattern. For example, a standard pattern recognition program can be used that sequentially detects edges and local intensity maxima in the received image; zooms in on the detected local intensity maxima; identifies intersection positions where the magnified local intensity maxima intersect with detected edges in the image; and zooms in on the identified intersection positions to define granule-like vesicle aggregation patterns induced by 4BAH2 peptide, wherein images of aggregates score higher than unaggregated vesicles. An example of such an analysis of a 384 well plate containing fluorescently-labelled vesicles, various small molecule compounds, and 4BAH2 is shown in FIG. 8.

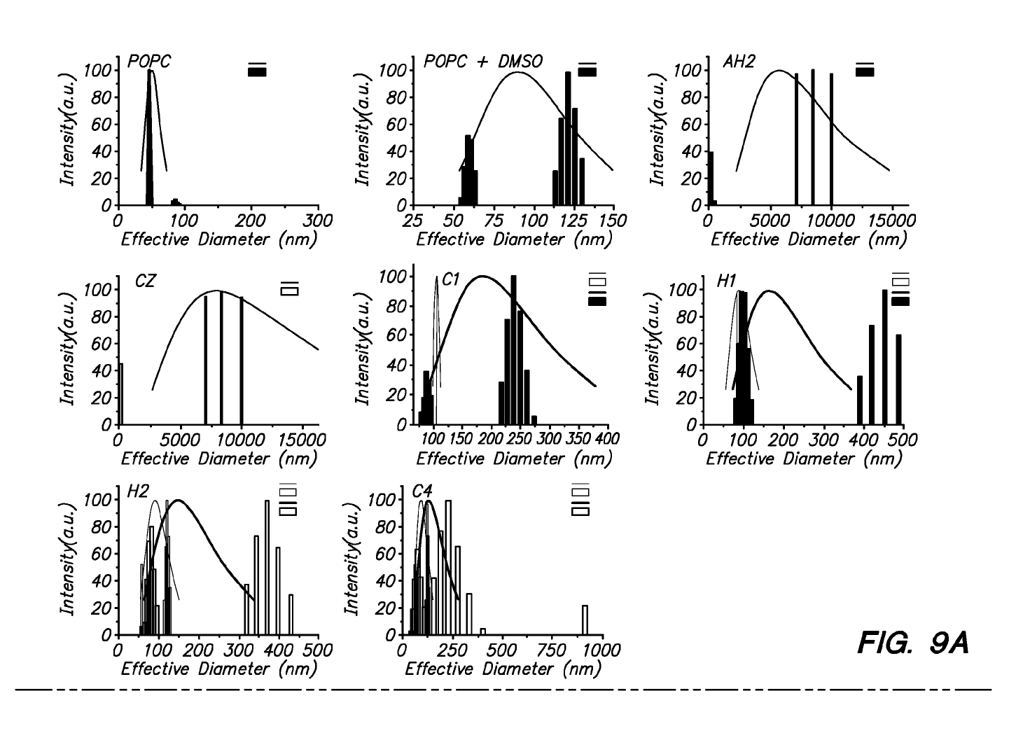

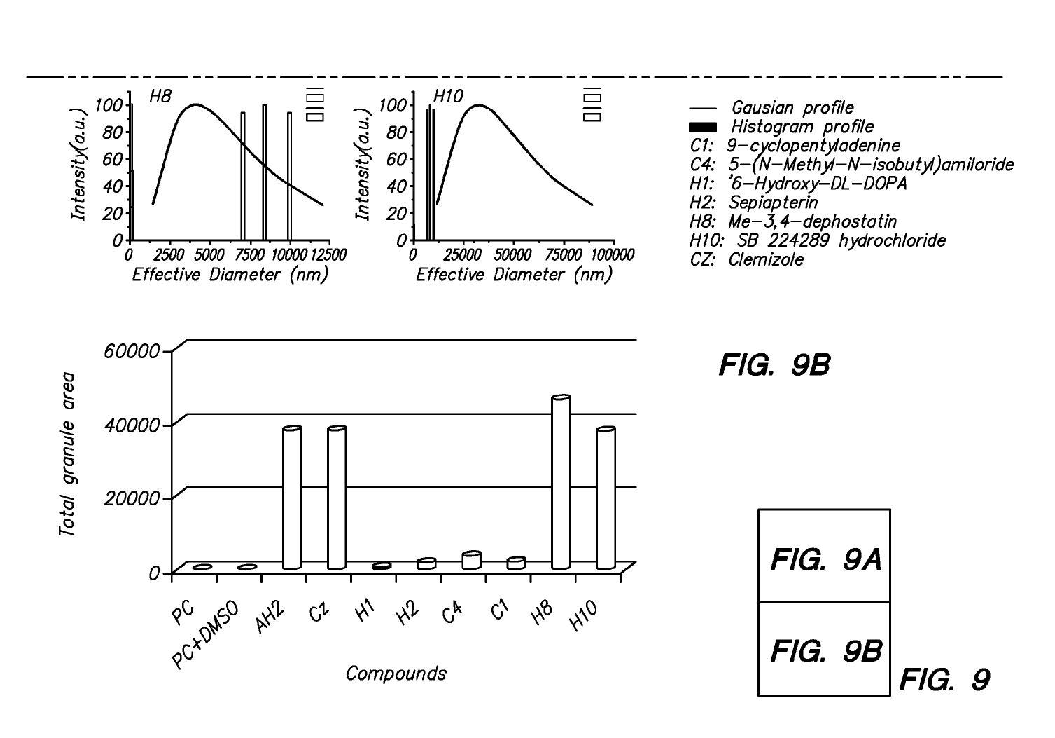

DLS assay on select hits of first screen. The above screen was performed on a collection of small molecules in a DMSO solution that was largely based on the Lopac library (Sigma). DLS was used to confirm the activity of the hits thus identified. As shown in FIG. 9, as expected all the hits were confirmed to be inhibitors of AH function and its ability to aggregate vesicles. Moreover, the DLS assay can be used in standard SAR efforts to identify more potent derivatives.

### Example 3

### Inhibitors of HCV AH Function Exhibit Antiviral Activity

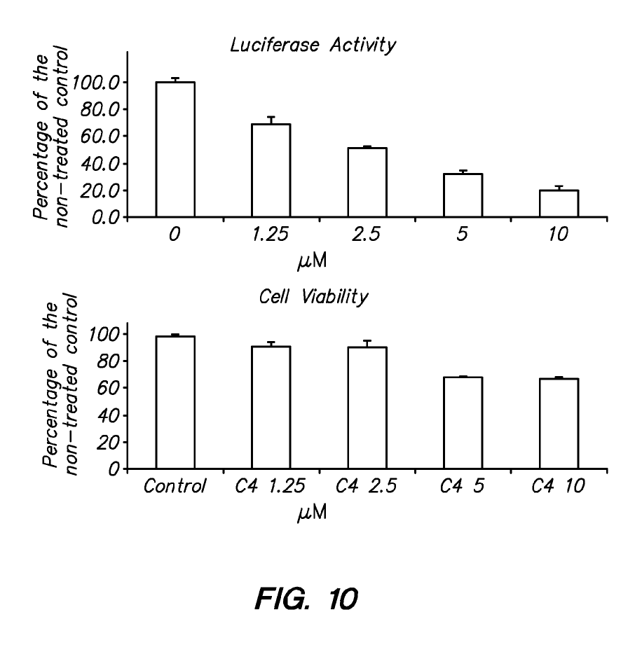

Effect of hits on HCV replication. Subsets of the above-identified hits are expected to be able to penetrate cells and similarly inhibit AH function within the context of the intact target protein in cells harboring replicating HCV genomes. An example of such a hit with antiviral activity against HCV is shown in FIG. 10. Compound C4 inhibits HCV replication in standard HCV replication assays: a genotype 1b luciferase reporter linked high efficiency subgenomic HCV replicon assay and Alamar blue assays for cell metabolism. Compound C4 increases the anti-HCV activity of NS3 protease inhibitor, SCH503034, “SCH”, that targets HCV (FIG. 11). Note that for the results shown in FIG. 11, a genotype 2a luciferase reporter-linked HCV replicons was used, indicating the broad spectrum potential of the C4 compound against multiple HCV genotypes.

### Materials and Methods

Dynamic light scattering. Dynamic light scattering was performed by a 90Plus particle size analyzer, and the results were analyzed by digital autocorrelator software (Brookhaven Instruments Corporation, New York). All measurements were taken at a scattering angle of 90°, where the reflection effect is minimized. Dynamic light scattering (DLS) is a well established technique for measuring particle size over the size range from a few nanometers to a few microns. The concept uses the idea that small particles in a suspension move in a random pattern, i.e., Brownian motion. When a coherent source of light (such as a laser) having a known frequency is directed at the moving particles, the light is scattered at a different frequency. The shift in light frequency is related to the size of the particles causing the shift. Due to their higher average velocity, smaller particles cause a greater shift in the light frequency than larger particles. It is this difference in the frequency of the scattered light among particles of different sizes that is used to determine the sizes of the particles present.

Vesicle aggregation assay. The AH2 peptide is responsible for aggregation of bilayer/vesicles upon interaction with solid substrate. Upon addition of AH2, vesicles massively aggregate and form aggregate structures on the plates. The vesicles are fluorescently labeled with Texas red and can be visualized using the ImageXpress Micro. Compounds that block the ability of AH2 to induce vesicle aggregation can be identified by visualizing the lack of AH2-induced aggregation of the fluorescently-labeled vesicles. In this assay compounds were added to 6.5˜13 uM AH2 peptide (final concentration) and then vesicles were added (0.125 mg/ml). Compound plates were set out to thaw 1 hour before assay. 30 μl_{—} of the AH2 Peptide Mix was added to columns 1 to 22 of the 384 well assay plates. 100 nL of compounds were transferred to the assay plates. 10 μL of the Vesicle mix was added to each well. Plates were centrifuged and then imaged to quantify vesicle aggregation formation after sealing to prevent the samples from drying out.

### Reagent List:

| | | | | Lot | |
|---|---|---|---|---|---|
| | | Vendor | | Number/ | |
| | CAS | (Manu- | Item | Date | |
| Reagent | Number | facturer) | Number | Made | Misc. |
| AH2 | | | | MW | 27 Amino |
| Peptide | | | | 3800 | acids |
| Vesicles | | Hand made | | | |
| | | Extrusion/ | | | |
| | | Avanti | | | |
| Assay | | PBS/DMSO | | | |
| Buffer | | | | | |
| (see | | | | | |
| below) | | | | | |
| Pin | | V&P | VP 110 | 30 mL & | Phosphoric |
| Cleaning | | Scientific | | 120 mL | Acid |
| Solution | | | | ddH2O | |
| Methanol | 67-56-1 | Fisher | A433P-4 | 032008-36 | |

### Buffers (Stock Solutions):

| | Final Concentration |
|---|---|
| Assay Buffer (500 mL for 30 plates) | |
| (50 mL) 100 mM PBS | 10 mM PBS pH 7.5 |
| (50 mL) 1500 mM NaCl | 150 mM NaCl |
| (400 mL) ddH2O | |
| 500 mL pH 7.5 Total Volume | |
| AH2 Peptide Mix (350 mL for 30 plates) | |
| (116.7 mL) 13 μM AH2 Peptide | 3.25 μM AH2 Peptide |
| (233.3 mL) Assay Buffer | 1× Assay Buffer |
| (350 mL) Total Volume | |
| uses 10.56 mL per plate, 30 μL per well, keep on | |
| ice | |
| Vesicle Mix (130 mL for 30 plates) | |
| (13 mL) 5 mg/mL Vesicles | 0.125 mg/mL Vesicles |
| (117 mL) Assay Buffer | 1× Assay Buffer |
| (130 mL) Total Volume | |
| uses 3.84 mL per plate, 10 μL per well, keep on | |
| ice | |

### Equipment & Materials List:

| Equipment | Vendor | Item Number/ | Lot/Serial | |
|---|---|---|---|---|
| Name | (Manufacturer) | Model Number | Number | Misc. |
| Twister II | CaliperLS | 79838/7 | T20407N0068 | |
| SciCloneALH | CaliperLS | ALH3000 | SS0407R4317 | |
| 3000 | | | | |
| 384 Pin Tool | V&P Scientific | AFIX384FP3H | BMPZYMARK | Hydrophobic pin |
| 100 nL | | Floating Tube, | | 0.787 Diameter |
| | | FP3 | | |
| ALHLow volume | CaliperLS | 103801 | SS0405N4294 | |
| EZ-Swap head | | | | |
| Air Compressor | Jun-Air | model 3-4 | 559790 | Set to 90 PSI |
| Microscan 710 | CaliperLS | 76709 | 0408957 | |
| Barcode Scanner | | | | |
| Multidrop 384- | Titertek | 5840200 | 32003965 | For adding vesicles |
| Staccato | | | | |
| ImageXpress | Molecular | IXMicro | 122639 | |
| Micro | Devices | | | |
| TRITC | Semrock | FF01-560/25 | | Excitation Filter |
| TRITC-FIXED | Semrock | FF01-607/36 | | Emission Filter |
| | | | | Cube |
| Quadband | Semrock | FF410/504/582/ | | |
| Dichroic | | 669-Di01 | | |
| Centrifuge | Beckman | Allegra-6 | AL599317 | |
| Clear-bottom 384 | E&K Scientific | EK-30091 | | Black walled |
| well plates | Greiner | (781091) | | polystyrene |
| Lint Free Blotting | V&P Scientific | VP 540D | | |
| Media | | | | |
| Polypropylene pad | V&P Scientific | VP 540DB1 | | |
| Omni Tray | V&P Scientific | VP 540DB | | |
| PlateLoc | Velocity11 | 01867.001 | 1.00406 | For sealing |
| | | | | compound plates |
| BenchCel 4X | Velocity11 | 08344.004 | 20.00158.0040 | |
| WellMate | Matrix | 201-10001 | 119542592 | |
| Dispenser | | | | |
| WellMate Stacker | Matrix | 201-20001 | 201-2-0107 | |
| WellMate Tubing- | Matrix | 201-30002 | | |
| Small Bore | | | | |
| Multidrop 384 | Titertek | 5840200 | 832003819 | |

### Example 4

### Identification of a Novel Class of HCV Inhibitors

All positive strand RNA viruses replicate their genome in intimate association with host intracellular membranes. Some viruses exploit the surface of pre-existing vesicular membranes such as endosomes. Other viruses, like HCV, induce the formation of novel membrane structures that represent the platform for membrane-associated RNA replication. In the case of HCV, the latter is believed to be derived in part from the endoplasmic reticulum and is termed the membranous web due to its appearance on electron microscopy consisting of aggregations of membranous vesicles.

Expression of the HCV NS4B protein alone has been reported to be sufficient for the creation of the membranous web, although the molecular mechanism(s) whereby NS4B might promote membrane rearrangements or vesicle aggregations that make up the membranous web are largely unknown. NS4B has four predicted transmembrane domains. An N-terminal amphipathic helix (AH) within NS4B mediates the targeting of the HCV replicase complex components to the apparent sites of replication and an arginine-rich like motif within NS4B binds the 3′-terminus region of the virus negative strand RNA, the presumed template for the initiation of progeny plus-strand RNA genomes.

Here we genetically validated a novel target within NS4B that is essential for enabling genome replication. This target consists of a second AH—termed 4BAH2—and was found to mediate oligomerization and lipid vesicle aggregation. We exploited this function to perform a high-throughput screen that identified a variety of small molecules capable of inhibiting 4BAH2-mediated lipid vesicle aggregation and HCV RNA replication. Detailed analysis of selected inhibitors by quartz crystal microbalance with dissipation (QCM-D) and atomic force microscopy (AFM) led to the identification of their mechanism of action. These results highlight 4BAH2 as a critical determinant of NS4B function, provide new insight into the molecular mechanism of HCV replication platform assembly, and demonstrate the feasibility of a novel small molecule anti-HCV strategy.

### Results

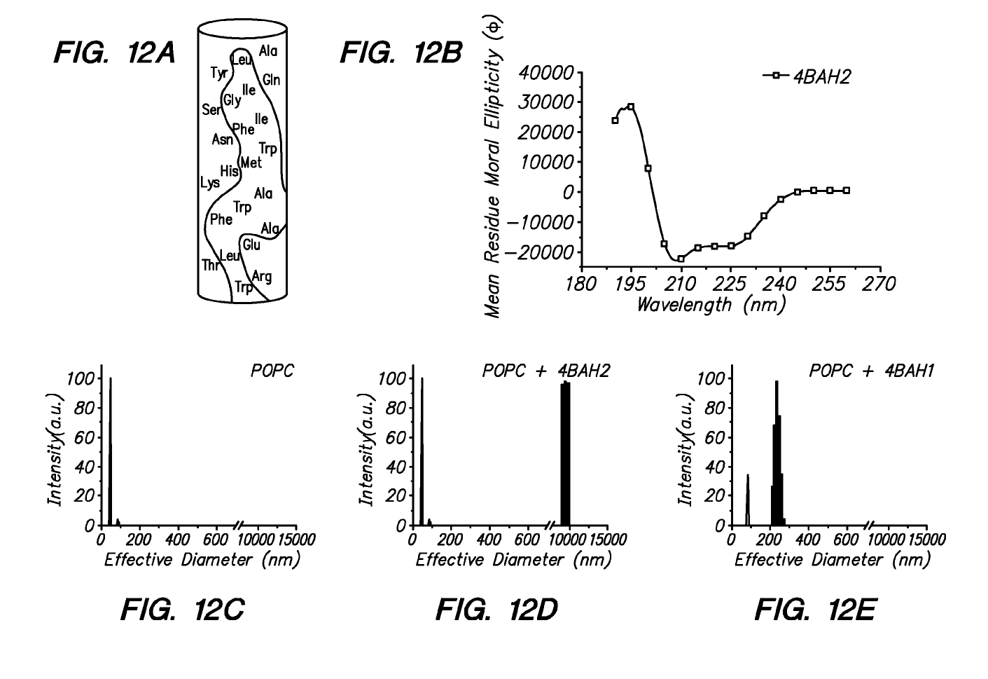

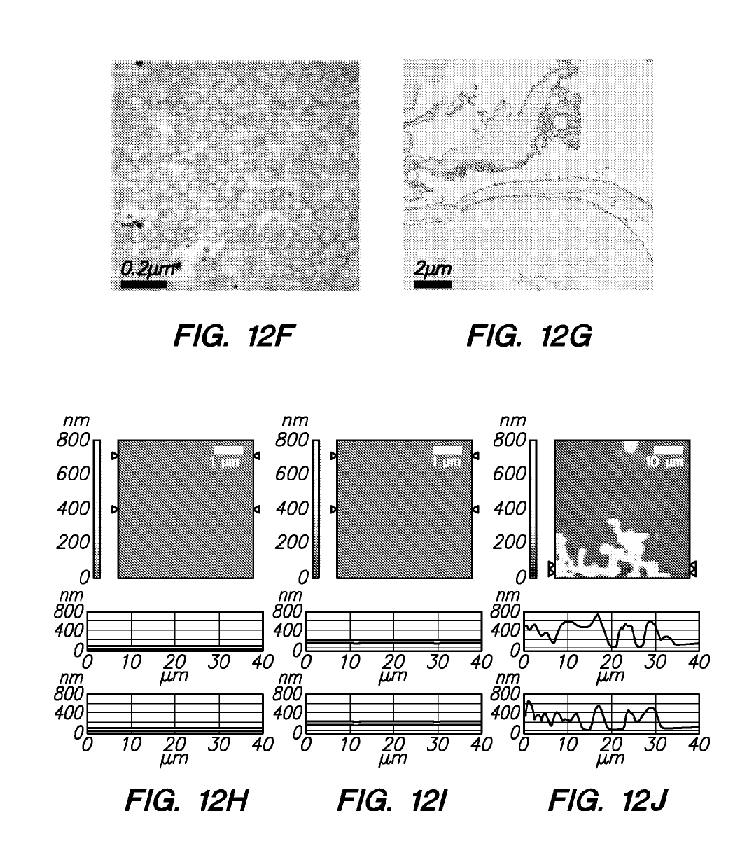

Amino acids 40 to 62 of NS4B comprise an amphipathic alpha helix (4BAH2). Secondary structure prediction programs (including DSC, HNNC, SIMPA96, MLRC, SOPM, PHD, and Predator) indicated that amino acids 40 to 62 of NS4B are likely to reside in an alpha helical conformation. Inspection of this helix revealed it to be amphipathic in nature (FIG. 12A). Because this segment is immediately downstream of another amphipathic helix, we defined the former as 4BAH2, and the more N terminal amphipathic helix as 4BAH1. As shown in FIG. 12B, circular dichroism (CD) measurements confirmed the helical nature of a synthetic peptide corresponding to 4BAH2.

4BAH2 induces vesicle aggregation. Expression of NS4B has been reported to be necessary and sufficient for induction of a novel intracellular membrane structure termed the membranous web that is believed to represent the platform upon which membrane associated HCV replication occurs. The membranous web derives its name by virtue of its appearance on electron microscopy, consisting of collections of membranous vesicle-like structures. To test the hypothesis that 4BAH2 might play a role in the formation of these membrane structures, we studied the interaction of 4BAH2 with 1-palmitoyl-2-oleoyl-snglycero-3-phosphocholine (POPC) lipid vesicles. The latter were selected because phosphocholine is most abundant in the endoplasmic reticulum (ER) and has a gel-fluid phase transition temperature (˜−10° C.) well below the experimentally convenient temperature of 24° C. Dynamic light scattering (DLS) indicated that the untreated extruded POPC vesicles had a relatively uniform size distribution, as shown in FIG. 12C. The average POPC vesicle diameter was 49.5±1.4 nm and the relative variance (polydispersity) of the vesicles was 0.118±0.02. The 4BAH2 peptide was then added to the lipid vesicles, while monitoring by DLS. A strikingly large increase in the average size of the vesicle population was observed (FIG. 12D). As shown in FIG. 12E, no such activity was observed with a control amphipathic helical peptide (4BAH1), highlighting the unique, specific, and striking biochemical activity associated with 4BAH2.

To determine whether this dramatic increase in size detected by DLS was due to either vesicle fusion or vesicle aggregation, we performed transmission electron microscopy on the vesicles before (FIG. 12F) and after (FIG. 12G) addition of 4BAH2. Most vesicles appear to retain their initial size, indicating that they are predominantly organized into large aggregates upon addition of 4BAH2. Note the extremely large size of the 4BAH2-induced aggregations, as indicated by the size calibration bar. Indeed, visual inspection showed that an initially clear solution of lipid vesicles was transformed almost instantaneously into one harboring large visible white aggregates upon addition of 4BAH2. To further confirm this apparent 4BAH2-induced aggregation of lipid vesicles, we also employed atomic force microscopy to follow the morphological changes associated with the addition of 4BAH2 to lipid vesicles upon interaction with a solid support. We used the hydrophilic SiOₓ substrate (FIG. 12H) as a supporting material since it is atomically flat and it is well known that vesicles typically fuse upon interaction with such hydrophilic substrates to make a ˜5 nm thin bilayers. Upon addition of vesicles alone, the featureless, uniform thickness of a ˜4.5 nm bilayer was observed (FIG. 12I). As expected, upon deposition of vesicles in the presence of 4BAH2, we detected massive 4BAH2-induced vesicle aggregates, as depicted in FIG. 1J and the corresponding line scan.

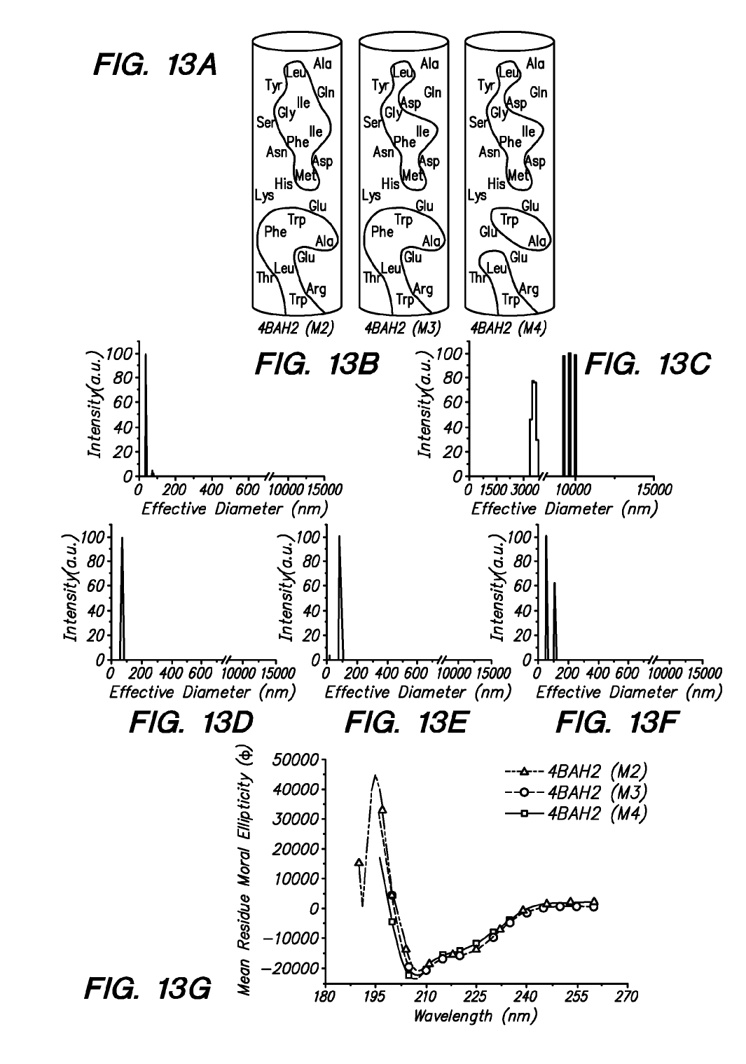

Disruption of 4BAH2's amphipathic nature abrogates vesicle aggregation. To test the hypothesis that the amphipathic nature of 4BAH2 was necessary for its vesicle aggregating activity, we repeated the experiments of FIGS. 12C and 12D using 4BAH2 peptides harboring the point mutations indicated in FIG. 13A, which were designed to disrupt 4BAH2's amphipathic nature. While wild-type 4BAH2 again induced dramatic aggregation of the lipid vesicles (FIG. 13C), disruption of 4BAH2's amphipathic nature completely abrogated its vesicle-aggregating activity (FIGS. 13D to 13F). To confirm that these results were not simply the result of mutation-induced loss of helical conformation, the CD studies of FIG. 2G were performed. These results indicated that the mutant 4BAH2 peptides retained their helical nature, and that it was their loss of amphipathicity that appeared to be a key determinant of their loss of vesicle-aggregating function. Similar results were obtained with AFM. Moreover, the mutations did not appear to alter NS4B stability.

An intact 4BAH2 is required for HCV genome replication. To test the hypothesis that an intact 4BAH2 is essential for viral genome replication, the least drastic mutations of FIG. 13A (corresponding to 4BAH2(M2)) were introduced into a bicistronic high efficiency HCV replicon (20) modified so that the HCV internal ribosome entry site (IRES) drives the expression of luciferase, and the non-structural proteins required for replication remain expressed under the encephalomyocarditis virus (EMCV) IRES. Wild-type and mutant replicons were then assayed in transient replication assays, along with a negative control mutant replicon with a lethal mutation in the NS5B polymerase gene. As shown in FIG. 3B, disruption of 4BAH2 abrogated genome replication. To confirm the dependence of HCV replication on 4BAH2, analogous replicons wherein the luciferase gene was replaced with the neomycin phosphotransferase gene were assayed in standard colony formation assays (FIG. 3A). Whereas the wild-type replicon yielded numerous colonies, no colonies resulted upon electroporation of the 4BAH2 mutant replicon. Together, these results demonstrate that mutations impairing the vesicle aggregating activity of 4BAH2 abrogate, and an intact 4BAH2 is required for, HCV genome replication.

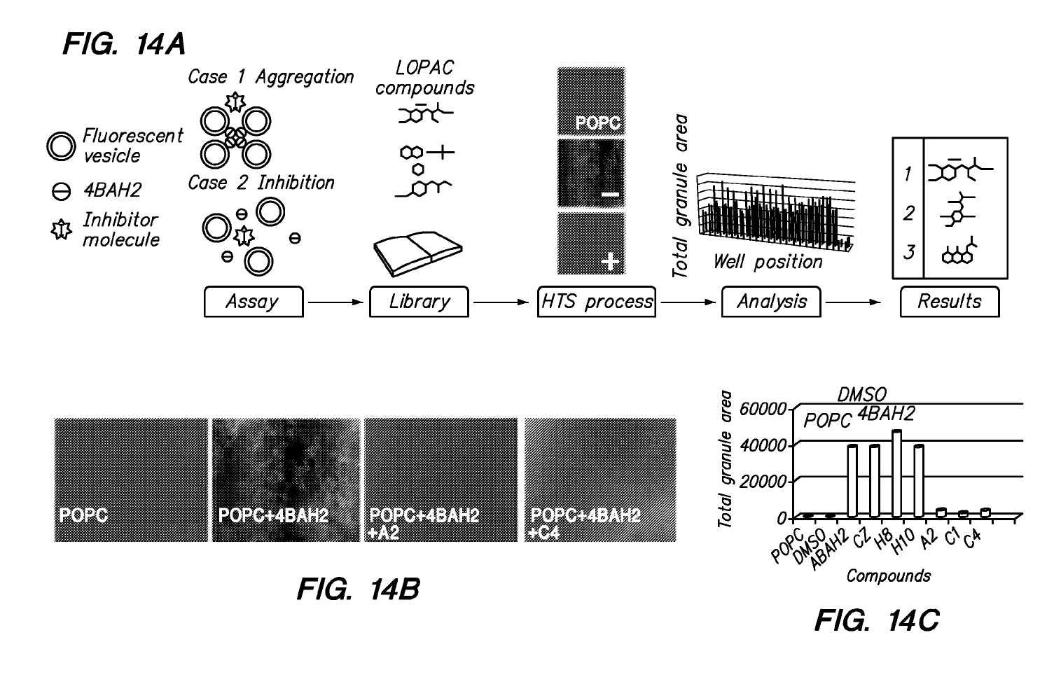

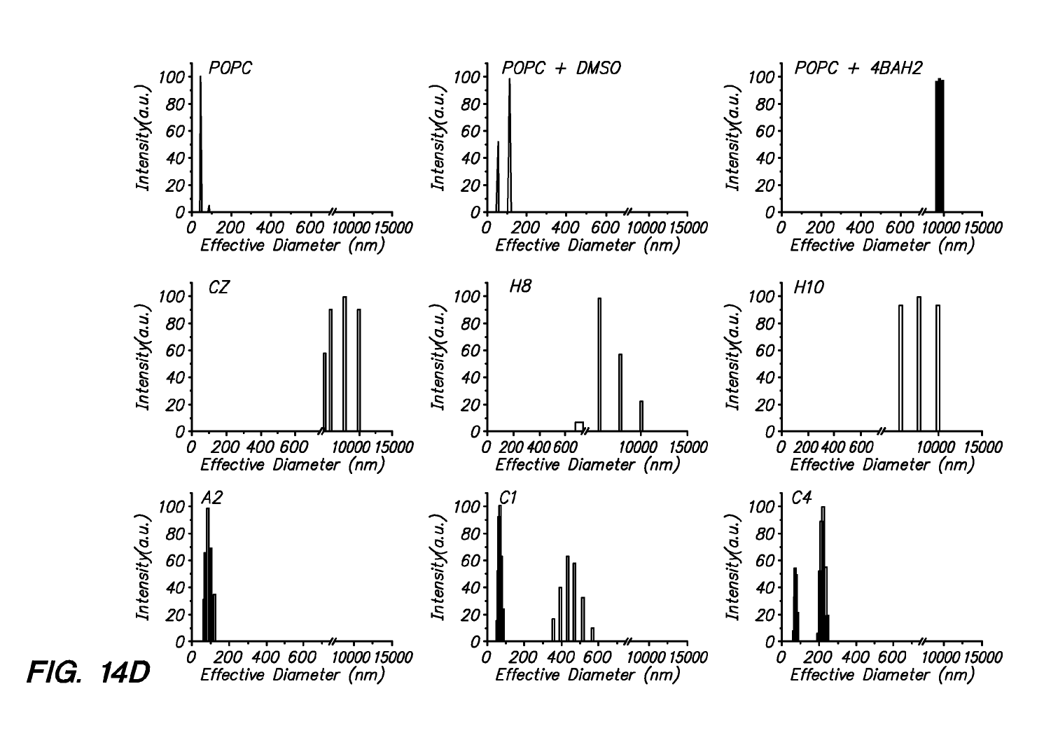

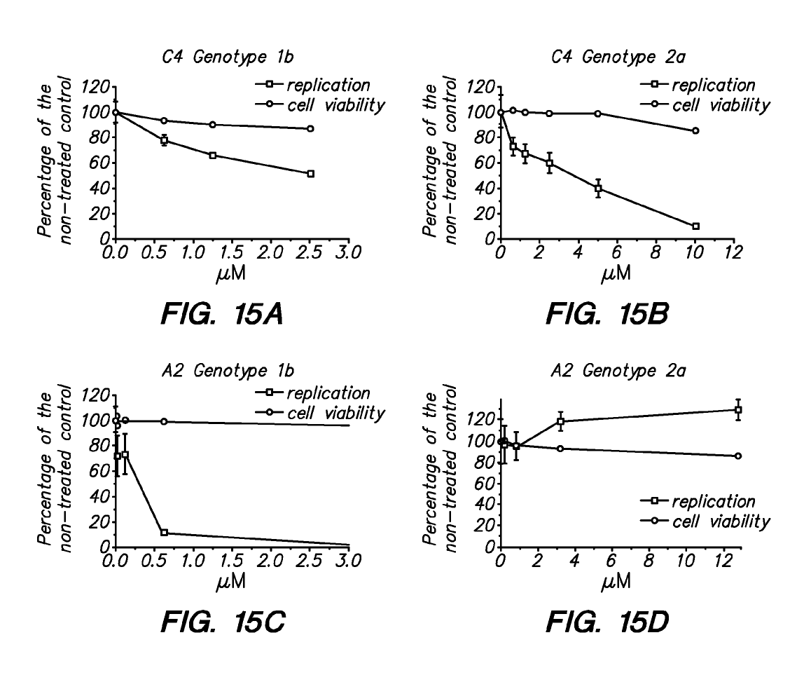

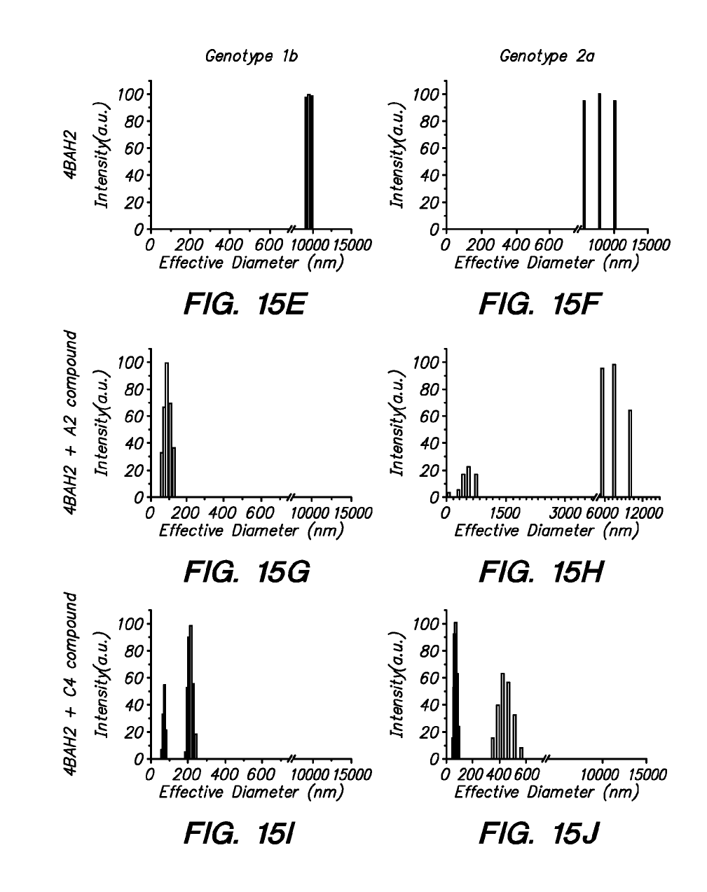

Identification of small molecule inhibitors of 4BAH2. The above results genetically validated the importance of 4BAH2 for HCV genome replication. As outlined in FIG. 4A, they also suggested an approach to identify pharmacologic inhibitors of 4BAH2 function. For this, POPC vesicles were labeled by incorporation of a fluorescent lipid (Texas red DHPE) and the vesicle-aggregating activity of added 4BAH2 peptide was monitored by fluorescence microscopy. The assay was adapted to a 384-well format and performed in the presence of compounds available from a small molecule library. 4BAH2-induced vesicle aggregates were imaged by automated fluorescence microscopy. Although it could be readily determined by visual inspection (FIG. 15B), the presence or absence of aggregates was analyzed using pattern recognition software in a high-throughput scheme (FIG. 4C). While most compounds had no significant effect on the vesicle-aggregating activity of 4BAH2, several inhibited aggregation formation to the background level observed with no addition of 4BAH2. These hits, along with selected compounds that displayed no inhibition of lipid vesicle aggregation and that were used as negative controls, were then further evaluated in a secondary screen in which DLS assays were performed similar to FIG. 13C in the presence of the individual compounds. As shown in FIG. 14D, several of the hits were confirmed to be quite potent inhibitors of 4BAH2's lipid vesicle-aggregating activity, and we a subset of these might similarly inhibit HCV genome replication.

Effect of selected hits on HCV replication and genotype specificity. The above hypothesis was tested in transient replication assays similar to those of FIG. 3B except for the presence of various concentrations of one of two compounds—C4 and A2—that demonstrated potent inhibition of 4BAH2-mediated vesicle aggregation. As shown in FIG. 15, both compounds exhibited dose-dependent inhibition of HCV replication. No significant cellular toxicity was observed under any of these conditions, highlighting the specificity of inhibition of HCV replication.

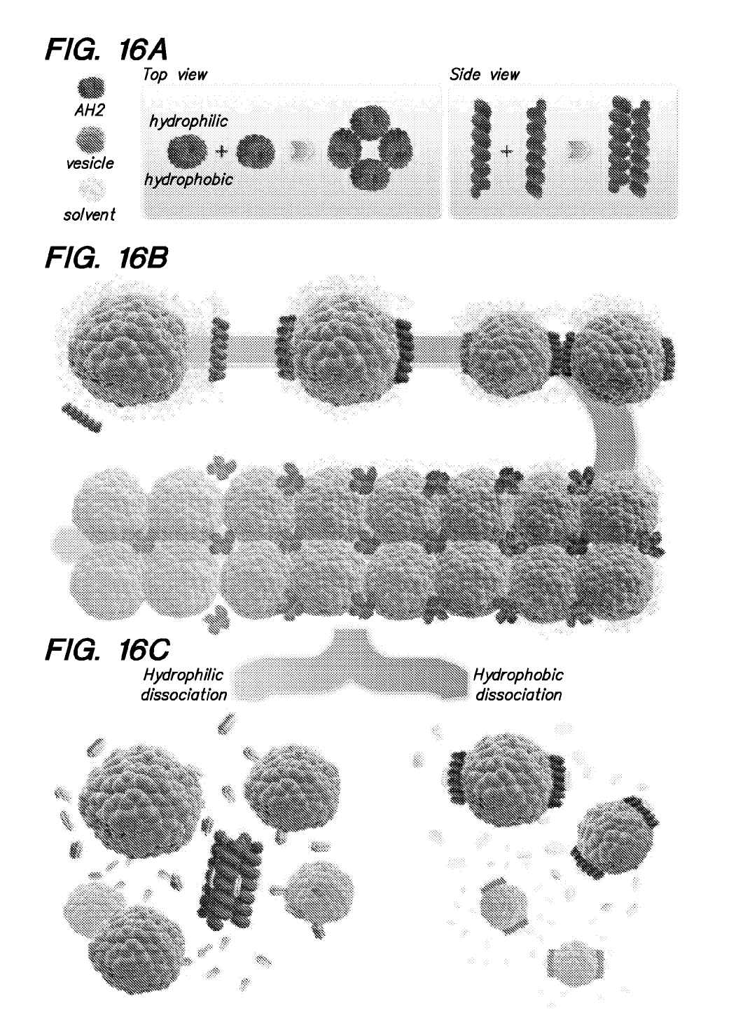

Recently, an infectious clone of HCV has been described, but which is of a different genotype (genotype 2a) than the genotype 1b replicons of FIG. 3. While both compounds inhibited genotype 1b replication (FIGS. 16A and 16C), only C4 exhibited inhibition of genotype 2a viral genome replication (FIG. 15B). We hypothesized that this reflected a difference in the specificity of the compounds for the 4BAH2 peptides of the respective genotypes.

To verify this, we determined the effect of the compounds in the DLS assay of FIG. 12D, except that genotype 2a 4BAH2 was used. As shown in FIG. 15F, in the absence of compound, 4BAH2 of genotype 2a induced a similar aggregation of lipid vesicles as did 4BAH2 of genotype 1b (FIGS. 16E, 12D, and 15D). However, while C4 essentially abrogated genotype 2a 4BAH2-induced vesicle aggregation (FIG. 15J), A2 had no significant effect (FIG. 15H). These results parallel the inhibitory effects of the compounds on replication of the respective genotypes (FIGS. 16A to 16D), and highlight the 4BAH2 specificity of the two compounds.

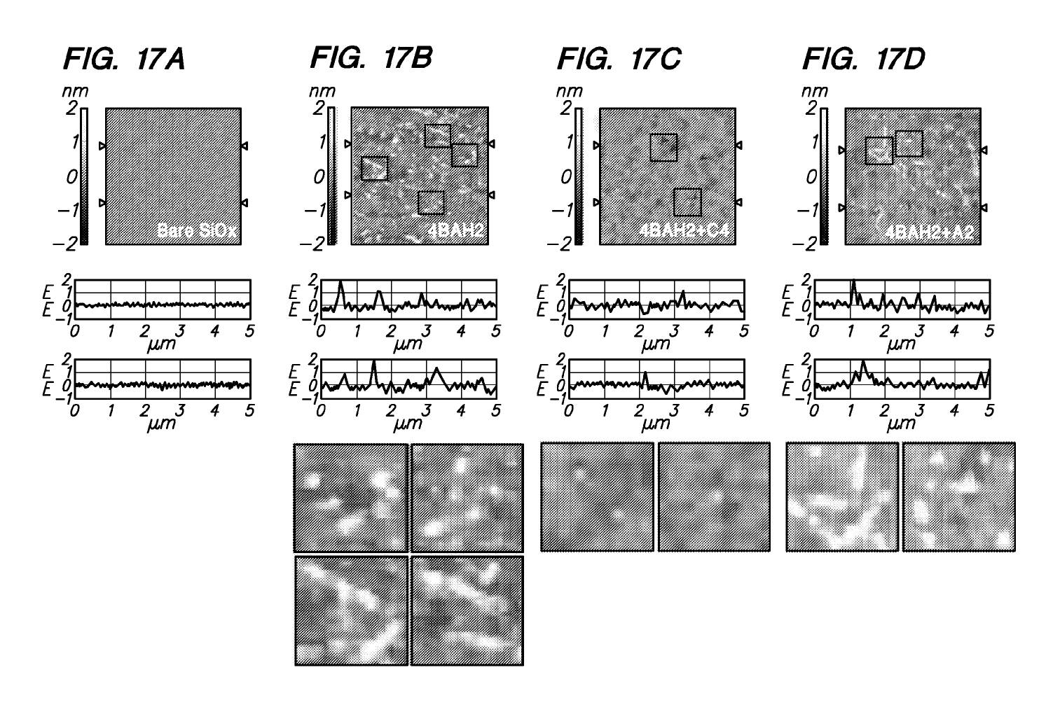

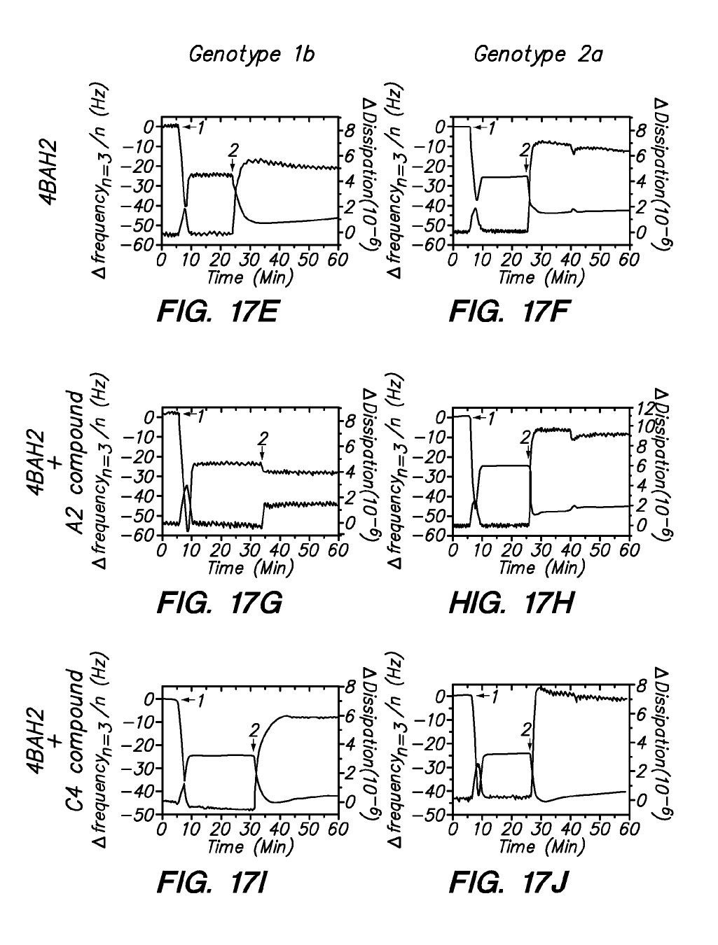

We envisage at least two possible mechanisms whereby 4BAH2-induced lipid vesicle aggregation can be inhibited: 1) preventing the ability of 4BAH2 peptides to oligomerize with each other, and 2) disrupting 4BAH2's ability to interact with lipid vesicles (see model, FIG. 16). To determine which of these mechanisms might be employed by the C4 and A2 compounds, we performed a variety of biophysical measurements designed to directly assess the effect of these compounds on 4BAH2 oligomerization (as detected by atomic force microscopy (AFM)) and membrane association (as monitored by quartz crystal microbalance-dissipation (QCM-D)). AFM provides for a quantitative assessment of surface topology and measurement of particle sizes. The QCM-D technique is ideal for studying the association of macromolecules with membranes coating the oscillating quartz crystal. Changes in resonance frequency are inversely proportional to the change in bound mass. Energy dissipation changes provide information about the associated ligands' oligomerization state by detecting their viscoelastic properties. The combined AFM and QCM-D data of FIG. 17 suggest that C4 acts primarily via disruption of 4BAH2 oligomerization, whereas A2's predominant effect is to prevent 4BAH2's interaction with membranes. In particular, there is prominent self-oligomerization of 4BAH2 peptides in the absence of inhibitor (FIG. 17B) whereas self-oligomerization is dramatically inhibited in the presence of C4 (FIG. 17C). The extent of inhibition was as great as that achieved by genetic mutations in 4BAH2 that completely abrogated 4BAH2 oligomerization. In contrast, addition of A2 had a relatively minimal effect on the ability of 4BAH2 to oligomerize (FIG. 17D) but completely prevented genotype 1b 4BAH2 membrane association (FIG. 17G). Again, A2's effect on 4BAH2 was limited to a genotype 1b target, with no significant inhibition of genotype 2a 4BAH2 membrane association (FIG. 17H). C4 had a minimal effect on the membrane association of either genotype's 4BAH2 (FIGS. 18I and 18J). The net effect of either C4 or A2, however, is to abrogate 4BAH2-mediated vesicle aggregation and membrane-associated HCV RNA genome replication that is dependent on the formation of the membranous web replication platform.

The limitations of current therapy for hepatitis C and the requirement for multi-drug cocktails to thwart the rapid development of resistance combine to highlight the need for new classes of HCV drugs. Here we genetically-validated a new target within the HCV NS4B protein, consisting of a conserved amphipathic helix (AH) that is essential for viral genome replication. We found this AH, termed 4BAH2, to have both the potential for self-oligomerization and a dramatic biochemical activity promoting the aggregation of lipid vesicles into macromolecular assemblies that display several key features of membranous webs—the HCV intracellular replication platform.

Furthermore, the 4BAH2 vesicle aggregation-promoting activity could be leveraged into a new screening assay for identifying candidate pharmacologic inhibitors. Several of the latter could inhibit HCV genome replication in a dose-dependent fashion. Moreover, the specificity of compounds for a particular HCV genotype could be further predicted by their ability to inhibit 4BAH2 function of the respective genotype. Detailed analysis of two of the latter compounds revealed that 4BAH2 function can be disrupted by either one of two mechanisms: inhibition of 4BAH2 oligomerization, or the ability of 4BAH2 to associate with membranes. These results suggest new insights into the mechanism of HCV's replication platform assembly, and identify a novel anti-HCV strategy.

The importance of 4BAH2 to the HCV life cycle is indicated by several lines of genetic evidence. First, a 4BAH2 is conserved across all HCV genotypes and isolates whose sequences are publicly available. This argues strongly for the dependence of productive viral replication in vivo on 4BAH2. Second, as shown by the transient replication assay of FIG. 3A, an HCV replicon harboring a genetically mutated 4BAH2 was defective in establishing genome replication. Third, similar genetic mutation of 4BAH2 resulted in the inability to maintain genome replication in the longer-term colony formation assays (FIG. 3B).

A molecular basis for 4BAH2's role in HCV replication was revealed by the oligomerization studies of FIG. 17 and the associated lipid vesicle-aggregating activity (FIG. 12D) that is dependent on 4BAH2 oligomerization. Oligomerization of NS4B has been reported by others, but a contribution of artefactual disulfide crosslinking post cell lysis could not be excluded. Here we found that point mutations within 4BAH2 that disrupted its amphipathic (FIG. 13A), but not helical (FIG. 13G), nature impaired the ability of NS4B to oligomerize. 4BAH2's oligomerization potential has direct relevance to the mechanism of the dramatic biochemical activity revealed in the course of studying 4BAH2's interaction with lipid vesicles. Indeed, as shown in FIGS. 12 and 13, the amphipathic helix 4BAH2 induces dramatic aggregation of lipid vesicles, defining a novel function within NS4B.

Although other amphipathic helices within HCV proteins are important for HCV replication and can also induce changes in apparent lipid vesicle size, as measured by DLS, the extent and mechanism of the DLS changes are different from that of 4BAH2. This is not too surprising given that the function of each of these AHs in the HCV life cycle is different. In addition to their common conserved amphipathic nature, they each have different and highly conserved sets of specific amino acids that likely mediate interactions specific to each AH. The magnitude of 4BAH2 induced changes in lipid vesicle size is striking and unique (FIG. 12D), and clearly distinguishes it from other HCV AH's studied to date. Electron microscopy confirmed that the increase in apparent vesicle size of up to two orders of magnitude reflects massive 4BAH2-induced aggregation of lipid vesicles.

Although a striking in vitro activity, our data suggest that 4BAH2-induced vesicle aggregation is also important for NS4B's role in the HCV life cycle. HCV replication is believed to occur in association with novel intracellular membrane structures induced by the virus. These structures, termed the membranous web, consist of aggregations of vesicles visualizable by electron microscopy. The newly-identified 4BAH2 vesicle-aggregating activity provides a mechanism to account for some of the key elements of the membranous web. The genetic validation data of FIG. 3 represents a critical first step in the targeted development of new potential HCV therapeutics. To efficiently translate such knowledge into new classes of HCV drugs, however, target-specific assays must be developed and understanding the mechanism of action of candidate inhibitors is needed.

The dramatic ability to induce vesicle aggregation suggested the basis for a high-throughput screen (HTS) to identify pharmacologic inhibitors of 4BAH2 function (FIG. 14). The HTS was readily adaptable to a 384 well plate format, and enabled identification of hits by either simple inspection of automatically acquired images, or by quantitative analysis of the latter using a pattern recognition program. (FIG. 14A). DLS provided a convenient secondary screening assay. As shown in FIG. 14D and as expected, all the hits were confirmed to be inhibitors of 4BAH2 function and its ability to aggregate vesicles. Moreover, the DLS assay can be used in standard structure-activity relationships (SAR) efforts to identify derivatives.

Subsets of the above-identified hits are expected to penetrate cells and similarly inhibit 4BAH2 function within the context of the intact target protein in cells harboring replicating HCV genomes. Examples of such compounds with antiviral activity against HCV are shown in FIG. 15. In the case of C4, its anti-HCV activity is not restricted to HCV genotype 1b, but rather it also exhibits activity against HCV genotype 2a, indicating that it has the potential for broad range efficacy against multiple HCV genotypes. In contrast, A2 is quite potent against HCV genotype 1b (the predominant genotype in the U.S.), yet ineffective against genotype 2a. Of note, similar efficacy patterns were demonstrated in the lipid vesicle aggregation assay (FIGS. 16E to 16J), where 4BAH2 peptides derived from genotypes 1b or 2a were equally effective at inducing vesicle aggregation. In particular, C4 could inhibit the 4BAH2 activity of either genotype, whereas A2 was only capable of inhibiting vesicle aggregation induced by the 4BAH2 derived from genotype 1b. These results provide additional validation for the specificity of the 4BAH2 assay.

One class of drugs currently in most advanced clinical development for hepatitis C is the NS5B polymerase inhibitors where inhibition of NS5B function can be achieved by targeting different facets of NS5B—including both the active site as well as several epitopes distinct from the active site. Similarly, our studies on the mechanistic details of how C4 and A2 inhibit 4BAH2 function suggest that the 4BAH2 class of inhibitors is also able to inhibit a common target by somewhat different mechanisms (see model FIG. 16). In particular, 4BAH2-mediated lipid vesicle aggregation depends on both 4BAH2's ability to oligomerize with itself, and 4BAH2's membrane binding ability. The AFM and QCM-D data of FIG. 17 combine to suggest that 4BAH2 inhibitors such as C4 predominantly target 4BAH2 oligomerization (i.e. 4BAH2-4BAH2 interactions), whereas 4BAH2 inhibitors like A2 appear to predominantly affect 4BAH2 membrane association. Either type of 4BAH2 inhibitor can inhibit HCV RNA genome replication. Not surprisingly, anti-HCV efficacy can be further increased when C4 is used in combination with other agents in development for hepatitis C. Moreover, the above oligomeric model of 4BAH2 suggests the potential for transdominant inhibition of 4BAH2 function.

In conclusion, we describe a novel activity of a key domain within the HCV NS4B protein—the 4BAH2 amphipathic helix—which may be employed to help establish the viral replication platform. Because disruption of this 4BAH2 function was lethal for HCV genome replication, 4BAH2 was genetically validated as a potential target for anti-HCV strategies. A novel 4BAH2 functional assay was established to identify small molecule pharmacologic inhibitors of 4BAH2. Importantly, these 4BAH2 inhibitors can prevent HCV RNA genome replication within cells. On a practical level, the 4BAH2 functional assay can predict the susceptibility of HCV genotypes to a given inhibitor, and enable the rapid establishment of structure-activity relationships for lead compound optimization. Thus compounds like C4 and A2 are expected to be useful probes of 4BAH2 function and its inhibition. They also represent a potential exciting novel class of compounds for inclusion in future cocktails that will almost certainly be necessary for effective pharmacologic control of HCV.

### Materials and Methods.

Small Unilamellar Vesicle Preparation. Vesicles of 1-palmitoyl-2-oleoyl-sn-glycero-3-phosphocholine (POPC) (Avanti Polar Lipids, Alabaster, Ala., USA) with well-defined size distributions were prepared by the extrusion method. Throughout the experiments, we used a phosphate buffered saline (PBS) buffer (10 mM, pH 7.5 and 150 mM NaCl) in 18.2 mΩ-cm MilliQ water (Millipore, Oregon, USA). Extruded vesicles (referred to simply as vesicles) were prepared in the following manner. Lipid films were prepared by first drying the as-supplied lipids dissolved in chloroform under a gentle stream of nitrogen at room temperature. Then the resulting lipid film was stored under vacuum for at least 5 hr in order to remove residual chloroform. Vesicles were prepared by first swelling the lipid film in aqueous solution then vortexing periodically for 5 min. The resulting vesicle solutions were subsequently sized by a mini extruder (Avanti Polar Lipids, Alabaster, USA) through polycarbonate membranes with nominal sizes of 100-nm, 50-nm and 30-nm pores. Vesicles were generally prepared at a nominal lipid concentration of ˜5 mg·ml-1, then subsequently diluted before experiments. Vesicles were generally used within a day of preparation.

Peptides. Peptides corresponding to the wild-type sequence of 4BAH2, as found in genotypes 1b (WRTLEAFWAKHMWNFISGIQYLA) and 2a, (WPKVEQFWARHMWNFISGIQYLA) were synthesized by Anaspec Corporation (San Jose, Calif., USA). For negative controls, three peptides harboring mutations in 4BAH2 (genotype 1b) were also synthesized by Anaspc Corporation.

Circular Dichroism. Circular dichroism (CD) measurements were carried out using an Aviv Model 215 equipped with a 450 watt Xenon arc lamp light source. CD scans in wavelength mode were recorded in the range of 190 nm to 270 nm at 1.0 nm steps and averaged over two scans. Measurements were carried out at 25° C. Spectral units were expressed as the molar ellipticity per residue by using peptide concentrations determined by measuring the UV light absorbance of tyrosine and tryptophan at 280 nm. The secondary scans were corrected for background based on blanks of PBS buffer containing 10 mM PBS, 250 mM NaCl, pH 7.5 with 50% (v/v) 2,2,2-trifluoroethanol (TFE). The scans obtained with ellipticity (O) were converted to mean molar residue ellipticity ([0]) as previously described. Spectra were processed with CD6 software, baseline corrected, and smoothed using a third-order least square polynomial fit.

Quartz Crystal Microbalance-Dissipation (QCM-D). Adsorption kinetics and the properties of the adsorbed layer were studied using a Q-Sense E4, multiple channel system (Q-Sense AB, Gothenburg, Sweden). The samples are introduced using a peristaltic pump with flow rate of 0.1 mL·min⁻¹. AT-cut quartz crystals (Q-Sense) of 14 mm in diameter coated with a SiOₓ layer were used for all vesicle interaction and adsorption experiments. Each QCM crystal was treated with oxygen plasma at ˜80 watts for 3 min prior to measurement (March Plasmod Plasma Etcher, March Instruments, California, USA). Each crystal was initially driven near its resonance frequency as indicated by a maximum in the current. To capture the characteristic dissipation, the drive circuit was short-circuited and the exponential decay of the crystal oscillation was recorded and analyzed, yielding the frequency and dissipation changes at 5, 15, 25, 35, 45, 55, and 65 MHz. The temperature of the Q-Sense cell was set at 25.0° C. and accurately controlled by a Peltier element in the cell with fluctuation smaller than ±0.05° C. All experiments were repeated at least three times, with a standard deviation of less than 2%.

High Throughput Screen (HTS). In order to screen for compounds that inhibit 4BAH2-mediated aggregation of nano-size vesicles, we performed a high-content imaging, high throughput screen (HTS). The assay was based on the 4BAH2 peptide's ability to induce large-scale aggregation of fluorescently-labeled vesicles that are readily detected by fluorescent microscopy. Texas Red-DHPE labeled, nanosize fluorescent lipid vesicles were prepared as described above, except for the addition of Texas Red-DHPE (added to a final molar ratio of POPC:Texas Red-DHPE of 99.5:0.5).

A Caliper Life Sciences Sciclone ALH3000 liquid handler integrated system (Stanford University High-Throughput Bioscience Center (HTBC)) was used to accommodate 384-tip manifolds, enabling it to rapidly pipet volumes into 384-well microplates. The Z8 module that contains eight independent syringe-based pipets, allowing liquid transfers with integrating a V&P Scientific 384 Pin Tool that is capable of 100 mL range transfers, was used. The sequence was as follows: A 384 well microplate was first retrieved from an automated incubator, the lid was removed, followed by the twister picking up the plate and taking it to a bar code reader. The microplate was then removed from the multidrop liquid dispenser and placed on a Sciclone deck. Appropriate volumes of each reagents/materials were transferred to microplates with the 384 Pin Tool. First, 4BAH2 peptide (final concentration of 6.5˜13 μM) was added and the fluorescently-labeled vesicles (final concentration 0.125 mg·m-1) were then added. After sealing the plates to preventing drying of the samples, the plates were then centrifuged and analyzed using a ImageXpress Microscope (Molecular Device) to quantify formation of vesicle aggregates.

Dynamic Light Scattering. Dynamic light scattering (DLS) was performed using a doubled, Nd:YAG laser (model 532 DPSS, Coherent Laser Group, Santa Clara, Calif.) with a wavelength, λ, of 633 nm and a Brookhaven digital autocorrelator, and analyzed by digital autocorrelator software (Brookhaven Instruments Corporation, New York, USA). Measurements of the intensity autocorrelation function were performed at a scattering angle of 90° using a linear spacing of the correlation time. DLS results were analyzed to give an intensity-weighted size distribution using a discrete Laplace inversion routine. All measurements were taken at a scattering angle of 90° where the reflection effect is minimized.

Atomic Force Microscopy. The AFM experiments were carried out on a XE-Bio (Park Systems Suwon, Korea) in contact and non-contact modes. Rectangular-shaped silicon cantilevers were used (SICON for contact mode and ACT for non-contact mode, AppNano, Santa Clara, Calif.). The cantilevers had a force constant of k=0.1 N/m for SICON and 25 N/m for ACT and an average tip radius of 5-6 nm. All measurements were performed in a PBS buffer. Images in fluid were obtained both in contact mode with an imaging force of less than 200 pN and in noncontact mode. However, images presented in this manuscript were only obtained in non-contact mode in fluid. The scan line speed was optimized between 0.3 Hz to 1 Hz with a pixel number of 256×256, depending on the scan size. Images were recorded in height, amplitude, phase, and error modes. All measurements were done on the height images. All images shown were subjected to a first order plane-fitting procedure to compensate for sample tilt. The cross-sectional analysis was carried out on images subjected only to a first order plane-fitting procedure. Topographical and grain analyses were performed using the software XEI 1.7.1 supplied by Park Systems (Suwon, Korea).

Transmission Electron Microscope (TEM). Samples were fixed in 4% glutaraldehyde (Electron Microsopy Sciences, Hatfield, Pa.) in 0.1 M cacodylate buffer pH-7, mixed well, then immediately 2% OsO₄ in 0.1 M cacodylate buffer pH-7 was added. Following incubation on ice for one hour, the fixed reaction was sedimented at 45000 rpm in a TLA100.3 rotor for 30 min at 4° C. The pellet was then refixed with 2% OsO₄ in 0.1M cacodylate buffer pH 7 for 30 min on ice, then washed three times with ultrafiltered water, followed by staining for 2 hr at room temperature or moved to 4° C. overnight. Samples were dehydrated in a series of ethanol washes for 15 min each at 4° C. beginning at 50%, then 70% and 95% when the samples were then allowed to rise to room temperature, and bathed two times at 100%. Samples were infiltrated with EMbed-812 resin (Electron Microsopy Sciences) mixed 1:1 with propylene oxide (PO) for 2 hr followed by EMbed-812 mixed 2:1 with PO overnight. The samples were subsequently placed into EMbed-812 for 2 to 4 hours, then placed into molds with labels and fresh resin, oriented and placed into a 65° C. oven overnight. Center sections were picked up on carboncoated mesh Cu grids, stained for 20 sec in 1:1 saturated uracetate (˜7.7%) in acetone, followed finally by staining in 0.2% lead citrate for 3 min. Samples were observed in a JEOL 1230 TEM at 80 kV and images were taken using a Gatan multiscan 791 digital camera.

Plasmids. Bart79I, a high-efficiency subgenomic replicon of HCV (28), harbors the neomycin resistance gene (neo) and the HCV nonstructural proteins. The Bart-Luciferase plasmid, Bart79I-luc, was cloned from the Bart791 parent (29) and the pGL3-Basic parent (Promega). In brief, a NotI site was introduced after the 15th amino acid of Core in Bart791 using PCR mutagenesis. The plasmid pcDNA3.1-NS4B, which encodes the Con1 NS4B sequence and was used to study the protein stability of the wild type and mutant NS4Bs, was described previously. To introduce the M2 mutation into the 4BAH2 of plasmids encoding HCV replicons or NS4B, the nucleotide sequence GCG that encodes for alanine at NS4B amino acid position 51 was changed to GAG (encoding for glutamate) and the nucleotide sequence TGG that encodes for tryptophan at amino acid position 55 was changed to GAT (encoding for aspartate) through the use of Quick-ChangeTMXL site-directed mutagenesis kit (Stratagene, La Jolla, Calif.) as described by the manufacturer and confirmed by sequencing. FL-J6/JFH-5′C19Rluc2AUbi, which is a monocistronic, full-length HCV genome that expresses Renilla luciferase (Rluc) and was derived from the previously described infectious genotype 2a HCV genome J6/JFH1.

Colony Formation Assays. 5 μg of in vitro transcribed wild type or mutant Bart79I RNAs were mixed with 6×10⁶ Huh7 cells in RNase-free PBS buffer (Biowhittaker) and transferred into a 2 mm-diameter gap cuvette (BTX, San Diego, Calif.). Electroporation was performed using a BTX model 830 electroporator. The electroporation condition was as follows: 680 V, five periods of 99 μs at 500 ms intervals. The electroporated cells were diluted in 10 ml of cell culture medium. Cells were transferred to 10 cm tissue culture dishes at different dilutions. At 24 hr post electroporation, cells were supplemented with untransfected feeder Huh7 cells to a final density of 10⁶ cells/plate. After an additional 24 hr, the medium was supplemented with G418 to a final concentration of 750 g·ml⁻¹. This selection medium was replaced every three days for three weeks. Following selection, the plates were washed with PBS buffer, incubated in 1% crystal violet in 20% ethanol for 5 min, and washed five times with H₂O.

Transient Replication Assays. 10 μg of in vitro transcribed wild type or mutant Bart79I-Luc RNAs were electroporated into Huh7 cells as described above. The electroporated cells were diluted in 40 ml of cell culture medium. 2 ml of cells were aliquoted in triplicate in 6 well tissue culture plates. Firefly luciferase activities were measured at 8, 48, 96, and 144 hr post electroporation by using a firefly luciferase kit from Promega (Madison, Wis.).

Antiviral Assays of Compounds. Subconfluent Huh7.5 cells were trypsinized and collected by centrifugation at 700 g for 5 min. The cells were then washed three times in ice-cold RNasefree PBS buffer (BioWhittaker) and resuspended at 1.5×10⁷ cells·ml⁻¹ in PBS buffer. Wild-type FL-J6/JFH-5′C19Rluc2AUbi and Bart79I-luc RNAs for electroporation were generated by in vitro transcription of XbaI (FL-J6/JFH-5′C19Rluc2AUbi) and ScaI (Bart791-luc)-linearized DNA templates using the T7 MEGAscript kit (Ambion), followed by phenol-chloroform purification and DEPC water suspension, 5 μg of RNA were mixed with 400 μl of washed Huh7.5 cells in a 2-mm-gap cuvette (BTX) and immediately pulsed (0.82 kV (FL-J6/JFH-5′C19Rluc2AUbi) and 0.68 kV (Bart79I-luc), five 99 ms pulses) with a BTX-830 electroporator. After 10 min recovery at 25° C., pulsed cells were diluted into 20 ml of pre-warmed growth medium. Cells from several electroporations were pooled to a common stock and seeded in 96-well plates (17,000-20,000 cells per well). After 24 hr, compounds were added to the cells and media changes were performed daily with fresh compounds. After 72 hr of treatment, cells were incubated for 2 hr at 37° C. in the presence of 10% Alamar Blue reagent (TREK Diagnostic Systems) to assess for cytotoxicity. Plates were then scanned and fluorescence was detected by using a FLEXstation II 384 (Molecular Devices). The signal was normalized relative to untreated samples. Viral RNA replication was determined using Renilla (FL-J6/JFH-5′C19Rluc2AUbi) or firefly (Bart791-luc) luciferase assays, according to the manufacturer's (Promega) directions. The same samples subjected to the viability assay described below were analyzed in this assay. According to the manufacturer protocol, cells were washed with PBS buffer and shaken at room temperature for 15 min in 20 μl of lysis buffer. Reporter assays were performed directly in the wells of the culture plates by injecting 100 μl of the assay substrate into each well. Luminescence was measured over 10 seconds with a 2-second delay using a Berthold LB 96 V luminometer. Signal was normalized relative to untreated samples or samples grown in the presence of the corresponding concentration of DMSO. Experiments were repeated three times, each time with 4 replicates.

Infection/transfection expression. A vaccinia virus that expresses the T7 RNA polymerase was used to infect Huh-7 cells at a multiplicity of infection of 10. Following a 45-minute incubation at 37° C., the cells were washed twice with Optimem (Invitrogen) and subjected to transfection with pcDNA3.1-NS4B wild type or pcDNA3.1-NS4B-AH2 (M2) mutant. The cells were supplemented with growth media and incubated for 22 hr at 37° C. Western blot analysis After infection/transfection expression, whole-cell extracts were prepared in RIPA buffer containing a cocktail of protease inhibitors (Complete, Mini; Roche Diagnostic) and quantitated by the Bradford assay (Bio-Rad). Equal amounts of protein were electrophoresed on an SDS-polyacrylamide gel, subsequently transferred to a polyvinylidene difluoride membrane (Immobilon-P; Millipore, Bedford, Mass.), and probed with rabbit anti-NS4B polyclonal antibody (32) (a gift from Dr. John McLauchlan, MRC Virology Unit, Institute of Virology, Glasgow G11 5JR, UK) with 1:500 dilution. Proteins were visualized via enhanced chemiluminescence (GE healthcare).

### Example 5

### Assay Procedure

The following procedure is used in the preparation of vesicles for assays of the invention. Prepare the following aqueous solutions to form vesicles: Tris/NaCl #1 (10 mM Tris, 100 mM NaCl, pH 7.5) is preferred for larger vesicles, such as 30-100 nanometer diameter, Tris/NaCl #2 (10 mM Tris, 250 mM NaCl, pH 7.5) is preferred for smaller diameter vesicles, such as those of 30-60 nanometer diameter and Tris/NaCl/CaCl₂ (10 mM Tris, 100 mM NaCl, 5 mM CaCl₂, pH 7.5). Use the Ca²⁺-containing buffer to form negatively charged bilayers and vesicles. The #4 PBS buffer may also be used. Filter all Tris buffers with 0.2 μm membrane before use.

Small unilamellar vesicle preparation by extrusion methods. Small unilamellar vesicles of 1-palmitoyl-2-oleoyl-sn-glycero-3-phosphocholine (Avanti Polar Lipids) were prepared by the extrusion method. Throughout the experiments, we used a Tris buffer, 10 mM Tris (pH 7.5) and 150 mM NaCl solution with 1 mM ethylene diamine tetraacetic acid (EDTA) in 18.2 Ωm MilliQ water (MilliPore). Lipid films were prepared by first drying the as-supplied lipids dissolved in chloroform under a gentle stream of nitrogen at room temperature. Then the resulting lipid film was stored under vacuum for at least 5 h in order to remove residual chloroform.

Multilamellar vesicles were prepared by first swelling the lipid film in aqueous solution then vortexing periodically for 5 min. The resulting multilamellar vesicles were subsequently sized by a miniextruder (Avanti Polar Lipids) through polycarbonate membranes with nominal 100 nm pores. The resulting multi- and uni-lamellar mixture vesicles were subsequently sized by a miniextruder (Avanti Polar Lipids) through polycarbonate membranes with nominal 50 nm pores again.

The resulting uni-lamellar vesicles were subsequently sized by a miniextruder (Avanti Polar Lipids) through polycarbonate membranes with nominal 30 nm pores again. Vesicles were generally prepared at a nominal lipid concentration of 5 mg·mL-1 and then subsequently diluted before experiments. Vesicles were generally used within 1 h of preparation.

AH Assay 1. Add synthetic peptide (NS4B-AH2) to vials. Add test compound to vials. Add prepared small unilamellar lipid vesicles of POPC (Avanti Polar Lipids). Centrifuge. Visualization of aggregation via visual inspection (yes/no) or dynamic light scattering reader (e.g. 90Plus NanoParticle Size Distribution Analyzer).

AH Assay 2. Add synthetic peptide (NS4B-AH2) to assay plates. Add test compound (with serial dilution) to assay plates. Add prepared small unilamellar lipid vesicles of POPC. Centrifuge Visualization of aggregation via fluorescence visual (e.g. ImageXpress) or dynamic light scattering reader (e.g. 90Plus NanoParticle Size Distribution Analyzer)

While the invention has been described in detail with reference to certain preferred embodiments thereof, it will be understood that modifications and variations are within the spirit and scope of that which is described and claimed.

1. A method for assessing activity of a candidate agent in interfering with a Hepatitis C virus (HCV) amphipathic helix (AH) peptide function, the method comprising:
contacting a suspension of lipid vesicles with a Hepatitis C virus (HCV) amphipathic helix peptide in the absence or presence of said candidate agent; and
determining a change in lipid vesicle size or aggregation, wherein an increase in vesicle size or aggregation is indicative of AH peptide function, and a lack of increase is indicative that said candidate agent is inhibiting AH peptide function.

2. The method of claim 1, wherein the HCV AH peptide is NS4B AH2 peptide.

3. The method of claim 1, wherein the HCV AH peptide is NS4B AH1 peptide.

4. The method of claim 1, wherein the HCV AH peptide is NS5A AH peptide.

5. The method of claim 1, wherein the peptide is (SEQ ID NO:16) WRTLEAFWAKHMWNFISGIQYLA.

6. The method of claim 1, wherein the peptide is amidated at the C terminus.

7. The method of claim 1, wherein the determining step is performed by detecting dynamic light scattering intensity signal.

8. The method of claim 1, wherein the determining step is performed by visual or automated inspection.

9. The method of claim 1, wherein the determining step if performed by fluorescence detection.

10. The method of claim 1, wherein the method is performed in a high throughput format.

11. A method of treating a hepatitis C virus (HCV) infection, the method comprising administering to an individual having an HCV infection an amount of a compound of the formula: 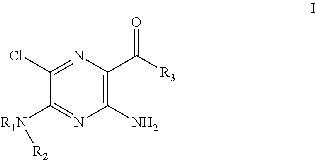
where R₁ and R₂ are independently selected from hydrogen; a lower C1-C6 alkyl, which may be branched or unbranched; or a benzyl; and
R₃ is NHR₄ or OR₄, where R₄ is selected from hydrogen, a lower alkyl, and CHR₅, where R₅ is selected from thiophene, isoxazole, thiazoles, pyridine, thiadiazole, benzene, cyclohexane, piperidine, and pyrrolidine, any of is optionally substituted with one or more substituents, including lower alkyl, halogen; carboxylic acid moiety.

12. The method of claim 11, wherein the compound has the formula:

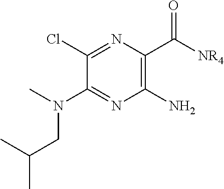

13. The method of claim 11, wherein the compound is selected from: 3-amino-N-carbamimidoyl-6-chloro-5-(isobutyl(methyl)amino)pyrazine-2-carboxamide; methyl 3-amino-6-chloro-5-(isobutyl(methyl)amino)pyrazine-2-carboxylate; 3-amino-6-chloro-5-(isobutyl(methyl)amino)pyrazine-2-carboxylic acid; 3-amino-6-chloro-5-(isobutyl(methyl)amino)-N-(thiophen-3-ylmethyl)pyrazine-2-carboxamide hydrochloride; 3-amino-6-chloro-5-(isobutyl(methyl)amino)-N-(thiazol-2-ylmethyl)pyrazine-2-carboxamide hydrochloride; 3-amino-6-chloro-5-(isobutyl(methyl)amino)-N-(thiophen-2-ylmethyl)pyrazine-2-carboxamide 2,2,2-trifluoroacetate; 3-amino-6-chloro-5-(isobutyl(methyl)amino)-N-(isoxazol-3-yl)pyrazine-2-carboxamide hydrochloride; 3-amino-6-chloro-5-(isobutyl(methyl)amino)-N-(pyridin-2-yl)pyrazine-2-carboxamide hydrochloride; 3-amino-6-chloro-5-(isobutyl(methyl)amino)-N-(pyridin-3-yl)pyrazine-2-carboxamide hydrochloride; 3-amino-6-chloro-5-(isobutyl(methyl)amino)-N-(pyridin-4-yl)pyrazine-2-carboxamide hydrochloride; 3-amino-6-chloro-5-(isobutyl(methyl)amino)-N-(pyridin-2-ylmethyl)pyrazine-2-carboxamide hydrochloride; 3-amino-6-chloro-5-(isobutyl(methyl)amino)-N-(1,3,4-thiadiazol-2-yl)pyrazine-2-carboxamide hydrochloride; 3-amino-6-chloro-5-(isobutyl(methyl)amino)-N-(4-methylpyridin-3-yl)pyrazine-2-carboxamide hydrochloride; 3-amino-6-chloro-5-(isobutyl(methyl)amino)-N-(5-methylisoxazol-3-yl)pyrazine-2-carboxamide hydrochloride; 3-amino-6-chloro-N-(6-chloro-5-methylpyridin-3-yl)-5-(isobutyl(methyl)amino)pyrazine-2-carboxamide bis(2,2,2-trifluoroacetate); 3-amino-N-benzyl-6-chloro-5-(isobutyl(methyl)amino)pyrazine-2-carboxamide 2,2,2-trifluoroacetate; 3-amino-6-chloro-N-(6-chloro-4-methylpyridin-3-yl)-5-(isobutyl(methyl)amino)pyrazine-2-carboxamide bis(2,2,2-trifluoroacetate); 3-amino-6-chloro-5-(isobutyl(methyl)amino)-N-(6-methoxy-4-methylpyridin-3-yl)pyrazine-2-carboxamide bis(2,2,2-trifluoroacetate); 3-amino-6-chloro-5-(isobutyl(methyl)amino)-N-phenylpyrazine-2-carboxamide; 3-amino-6-chloro-5-(isobutyl(methyl)amino)-N-(5-methylpyridin-3-yl)pyrazine-2-carboxamide bis(2,2,2-trifluoroacetate); 3-amino-6-chloro-N-(6-fluoro-4-methylpyridin-3-yl)-5-(isobutyl(methyl)amino)pyrazine-2-carboxamide bis(2,2,2-trifluoroacetate); 3-amino-6-chloro-5-(isobutyl(methyl)amino)-N-(piperidin-3-yl)pyrazine-2-carboxamide 2,2,2-trifluoroacetate; 3-amino-6-chloro-N-cyclohexyl-5-(isobutyl(methyl)amino)pyrazine-2-carboxamide 2,2,2-trifluoroacetate; 3-amino-6-chloro-5-(isobutyl(methyl)amino)-N-(piperidin-4-yl)pyrazine-2-carboxamide 2,2,2-trifluoroacetate; 3-amino-6-chloro-N-(cyclohexylmethyl)-5-(isobutyl(methyl)amino)pyrazine-2-carboxamide 2,2,2-trifluoroacetate; 3-amino-6-chloro-5-(isobutyl(methyl)amino)-N-(pyridin-3-ylmethyl)pyrazine-2-carboxamide 2,2,2-trifluoroacetate; 3-amino-6-chloro-5-(isobutyl(methyl)amino)-N-(pyridin-4-ylmethyl)pyrazine-2-carboxamide 2,2,2-trifluoroacetate; methyl 3-(3-amino-6-chloro-5-(isobutyl(methyl)amino)pyrazine-2-carboxamido)benzoate 2,2,2-trifluoroacetate; 5-(isobutyl(methyl)amino)-N-(4-methylpyridin-3-yl)pyrazine-2-carboxamide 2,2,2-trifluoroacetate; 3-amino-6-chloro-N-(4-methylpyridin-3-yl)-5-(piperidin-1-yl)pyrazine-2-carboxamide 2,2,2-trifluoroacetate; 3-amino-6-chloro-N-(4-methylpyridin-3-yl)-5-morpholinopyrazine-2-carboxamide 2,2,2-trifluoroacetate; 3-amino-6-chloro-N-(4-methylpyridin-3-yl)-5-(pyrrolidin-1-yl)pyrazine-2-carboxamide 2,2,2-trifluoroacetate; 3-amino-5-(benzyl(methyl)amino)-6-chloro-N-(4-methylpyridin-3-yl)pyrazine-2-carboxamide 2,2,2-trifluoroacetate; 3-amino-6-chloro-5-(diethylamino)-N-(4-methylpyridin-3-yl)pyrazine-2-carboxamide 2,2,2-trifluoroacetate; 3-amino-6-chloro-5-(isobutylamino)-N-(4-methylpyridin-3-yl)pyrazine-2-carboxamide 2,2,2-trifluoroacetate; 3-amino-6-chloro-5-(methyl(phenyl)amino)-N-(4-methylpyridin-3-yl)pyrazine-2-carboxamide 2,2,2-trifluoroacetate; 3-amino-6-chloro-5-(ethyl(methyl)amino)-N-(4-methylpyridin-3-yl)pyrazine-2-carboxamide 2,2,2-trifluoroacetate; and 6-chloro-5-(isobutyl(methyl)amino)-N-(4-methylpyridin-3-yl)pyrazine-2-carboxamide 2,2,2-trifluoroacetate
that is effective, when administered in one or more doses, to reduce HCV viral load in the individual.

14. The method of claim 13, wherein the compound is 3-amino-N-carbamimidoyl-6-chloro-5-(isobutyl(methyl)amino)pyrazine-2-carboxamide.

15. The method of claim 11, wherein the HCV viral load is reduced to below 10⁵ HCV genomes per milliliter serum.

16. The method of claim 11, wherein the compound is administered in an amount of from about 15 mg to about 100 mg per dose.

17. The method of claim 11, further comprising administering at least one additional anti-HCV therapeutic agent.

18. The method of claim 17, wherein the at least one additional therapeutic agent comprises an HCV NS3 protease inhibitor.

19. The method of claim 18, wherein the at least one additional therapeutic agent comprises an HCV NS5B RNA-dependent RNA polymerase inhibitor.

20. The method of claim 18, wherein the at least one additional therapeutic agent comprises a nucleoside analog.

21. The method of claim 18, wherein the at least one additional therapeutic agent comprises an interferon-alpha.

22. The method of claim 19, wherein the at least one additional therapeutic agent comprises clemizole or its analogs.

23. The method of claim 18, wherein the at least one additional therapeutic agent comprises nitazoxanide or another thiazolide.

24. The method of claim 18, wherein the individual is a human.


SEQUENCE LISTING (17 sequences)

SEQ ID NO: 1 - protein, 30 aa, Hepatitis C Virus
AVQTNWQKLEVFWAKHMWNFISGIQYLAGL

SEQ ID NO: 2 - protein, 30 aa, Hepatitis C Virus
VVESKWRTLEAFWAKHMWNFISGIQYLAGL

SEQ ID NO: 3 - protein, 30 aa, Hepatitis C Virus
AMQASWPKVEQFWARHMWNFISGIQYLAGL

SEQ ID NO: 4 - protein, 30 aa, Hepatitis C Virus
IVATNWQKLEAFWHKHMWNFVSGIQYLAGL

SEQ ID NO: 5 - protein, 30 aa, Hepatitis C Virus
VIQSNFAKLEQFWAKHMWNFISGIQYLAGL

SEQ ID NO: 6 - protein, 30 aa, Hepatitis C Virus
AATSMWNRAEQFWAKHMWNFVSGIQYLAGL

SEQ ID NO: 7 - protein, 30 aa, Hepatitis C Virus
AVHSAWPRMEEFWRKHMWNFVSGIQYLAGL

SEQ ID NO: 8 - protein, 30 aa, Hepatitis C Virus
VVESKWRTLEAFWAKHMWNFISGVQYLAGL

SEQ ID NO: 9 - protein, 30 aa, Hepatitis C Virus
VVESKWRTLETFWAKHMWNFISGIQYLAGL

SEQ ID NO: 10 - protein, 30 aa, Hepatitis C Virus; identical to SEQ ID NO: 9

SEQ ID NO: 11 - protein, 30 aa, Hepatitis C Virus
VVESKWRSLEAFWAKHMWNFISGIQYLAGL

SEQ ID NO: 12 - protein, 30 aa, Hepatitis C Virus; identical to SEQ ID NO: 11

SEQ ID NO: 13 - protein, 30 aa, Hepatitis C Virus; identical to SEQ ID NO: 11

SEQ ID NO: 14 - protein, 30 aa, Hepatitis C Virus; identical to SEQ ID NO: 9

SEQ ID NO: 15 - protein, 30 aa, Hepatitis C Virus
VVESKWRALEAFWAKHMWNFISGIQYLAGL

SEQ ID NO: 16 - protein, 23 aa, Hepatitis C Virus
WRTLEAFWAKHMWNFISGIQYLA

SEQ ID NO: 17 - protein, 30 aa, Hepatitis C Virus; identical to SEQ ID NO: 2